(19) United States
(12) Patent Application Publication
(10) Pub. No.: US 2011/0191910 A1
(43) Pub. Date: Aug. 4, 2011

(54) PLANTS WITH ALTERED ROOT ARCHITECTURE, RELATED CONSTRUCTS AND METHODS INVOLVING GENES ENCODING LECTIN PROTEIN KINASE (LPK) POLYPEPTIDES AND HOMOLOGS THEREOF

(75) Inventor: Graziana Taramino, Wilmington, DE (US)

(73) Assignee: E. I. DU PONT DE NEMOURS AND COMPANY, Wilmington, DE (US)

(21) Appl. No.: 13/018,521

(22) Filed: Feb. 1, 2011

Related U.S. Application Data
(60) Provisional application No. 61/300,490, filed on Feb. 2, 2010.

(51) Int. Cl. C12N 15/87; A01H 5/00; C07H 21/04; C12Q 1/68

(52) U.S. Cl. 800/290; 800/298; 800/320.1; 800/312; 800/322; 800/320; 800/320.3; 800/314; 800/320.2; 800/306; 536/23.1; 435/6.1

(57) ABSTRACT

Isolated polynucleotides and polypeptides and recombinant DNA constructs particularly useful for altering root structure of plants, compositions (such as plants or seeds) comprising these recombinant DNA constructs, and methods utilizing these recombinant DNA constructs. The recombinant DNA construct comprises a polynucleotide operably linked to a promoter functional in a plant, wherein said polynucleotide encodes a polypeptide useful for altering plant root architecture.

### FIELD OF THE INVENTION

The field of invention relates to plant breeding and genetics and, in particular, relates to recombinant DNA constructs useful in plants for altering root architecture.

### BACKGROUND OF THE INVENTION

Water and nutrient availability limit plant growth in all but a very few natural ecosystems. They limit yield in most agricultural ecosystems. Plant roots serve important functions such as water and nutrient uptake, anchorage of the plants in the soil and the establishment of biotic interactions at the rhizosphere. Elucidation of the genetic regulation of plant root development and function is therefore the subject of considerable interest in agriculture and ecology.

The root system originates from a primary root that develops during embryogenesis. The primary root produces secondary roots, which in turn produce tertiary roots. All secondary, tertiary, quaternary and further roots are referred to as lateral roots. Many plants, including maize, can also produce shoot borne roots, from consecutive under-ground nodes (crown roots) or above-ground nodes (brace roots). Three major processes affect the overall architecture of the root system. First, cell division at the primary root meristem enables indeterminate growth by adding new cells to the root. Second, lateral root formation increases the exploratory capacity of the root system. Third, root-hair formation increases the total surface of primary and lateral roots (Lopez-Bucio et al., Current Opinion in Plant Biology (2003) 6:280-287). In maize mutants have been isolated that are missing only a subset of root types. In Arabidopsis, mutations in root patterning genes such as SHORTROOT and SCARECROW, which show developmental defects in primary and lateral roots, have been identified (J. E. Malamy, Plant, Cell and Environment (2005) 28: 67-77).

A number of maize mutants affected specifically in root development have been identified (Hochholdinger et al 2004, Annals of Botany 93:359-368). The recessive mutants rtcs and rt1 forms no, or fewer, crown and brace roots, while the primary and lateral roots are not affected. In the recessive mutants des21, lateral seminal roots and root hairs are absent. Root hairs are lacking in the recessive mutant rth1-3. The mutants Irt1 and rum1 are affected before lateral root initiation and mutants slr1 and slr2 are impaired in lateral root elongation. Intrinsic response pathways that determine root system architecture include hormones, cell cycle regulators and regulatory genes. Water stress and nutrient availability belong to the environmental response pathways that determine root system architecture.

U.S. Application No. 2005-57473 filed Feb. 14, 2005 (U.S. Patent Publication No. 2005/223429 A1 published Oct. 6, 2005) concerns the use of Arabidopsis cytokinin oxidase genes to alter cytokinin levels in plants and stimulate root growth.

U.S. Pat. No. 6,344,601 (issued Feb. 5, 2002) concerns the under- or overexpression of profilin in a plant cell to alter plant growth habit, e.g. a reduced root and root hair system, delay in the onset of flowering.

US2004/016432 (filed May 21, 2004 (WO2004/106531 published Dec. 9, 2004) concerns the use of methods to manipulate the growth rate and/or yield and/or architecture by over expression of cis-prenyltransferase.

U.S. Application No. 2004/489500 filed Sep. 30, 2004 (U.S. Patent Publication No. 2005/059154 A1 published Mar. 13, 2005) concerns methods to modify cell number, architecture and yield using over expression of the transcription factor E2F in plants.

Activation tagging can be utilized to identify genes with the ability to affect a trait. This approach has been used in the model plant species Arabidopsis thaliana (Weigel et al., 2000, Plant Physiol. 122:1003-1013).

Insertions of transcriptional enhancer elements can dominantly activate and/or elevate the expression of nearby endogenous genes.

### SUMMARY OF THE INVENTION

The present invention includes:

In one embodiment, a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70 and wherein said plant exhibits altered root architecture when compared to a control plant not comprising said recombinant DNA construct.

In another embodiment a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, and wherein said plant exhibits an alteration of at least one agronomic characteristic when compared to a control plant not comprising said recombinant DNA construct. Optionally, the plant exhibits said alteration of said at least one agronomic characteristic, when compared, under varying environmental condition to a control plant not comprising said recombinant DNA construct and wherein said environmental condition is at least one selected from drought, nitrogen, soil type, insect and disease. The at least one agronomic trait may be yield, biomass, root lodging, or root architecture or any combination thereof.

In another embodiment, the present invention includes any of the plants of the present invention wherein the plant is selected from the group consisting of: maize, soybean, sunflower, sorghum, canola, wheat, alfalfa, cotton, rice barley, millet, sugar cane and switchgrass.

In another embodiment, the present invention includes seed of any of the plants of the present invention, wherein said seed comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70 and wherein a plant produced from said seed exhibits either an alteration in root architecture, or an alteration in at least one agromomic characteristic, or both, when compared to a control plant not comprising said recombinant DNA construct. The at least one agromomic trait may be root architecture, root lodging, yield or biomass or any combination thereof.

In another embodiment, a method of altering root architecture in a plant, comprising:

(a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70;

(b) regenerating a transgenic plant from the regenerable plant cell after step (a), wherein the transgenic plant comprises in its genome the recombinant DNA construct; and

(c) obtaining a progeny plant derived from the transgenic plant of step (b), wherein said progeny plant comprises in its genome the recombinant DNA construct and exhibits an alteration in root architecture when compared to a control plant not comprising the recombinant DNA construct.

In another embodiment, a method of evaluating alteration in root architecture in a plant, comprising:

(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70;

(b) obtaining a progeny plant derived from the transgenic plant, wherein the progeny plant comprises in its genome the recombinant DNA construct; and

(c) evaluating the progeny plant for alteration in root architecture compared to a control plant not comprising the recombinant DNA construct.

In another embodiment, a method of determining an alteration of at least one agromomic characteristic in a plant, comprising:

(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70;

(b) obtaining a progeny plant derived from the transgenic plant, wherein the progeny plant comprises in its genome the recombinant DNA construct; and

(c) determining whether the progeny plant exhibits an alteration of at least one agronomic characteristic when compared to a control plant not comprising the recombinant DNA construct. Optionally, said determining step (c) comprises determining whether the transgenic plant exhibits an alteration of at least one agronomic characteristic when compared, under varying environmental conditions, such as drought, nitrogen, soil type, insect or disease, to a control plant not comprising the recombinant construct.

In another embodiment, the present invention includes any of the methods of the present invention wherein the plant is selected from the group consisting of:

maize, soybean, sunflower, sorghum, canola, wheat, alfalfa, cotton, rice barley, millet, sugar cane and switchgrass.

In another embodiment, the present invention includes an isolated polynucleotide comprising:

(a) a nucleotide sequence encoding a polypeptide having an amino acid sequence of at least 85%, sequence identity, based on the Clustal V method of alignment, when compared to SEQ ID NO: 35 or

(b) a full complement of the nucleotide sequence, wherein the full complement and the nucleotide sequence consist of the same number of nucleotides and are 100% complementary. The polypeptide may comprise the amino acid sequence of SEQ ID NO: 35. The nucleotide sequence may comprise the nucleotide sequence of SEQ ID NO: 34.

In another embodiment, the present invention concerns a recombinant DNA

Construct comprising any of the isolated polynucleotides of the present invention operably linked to at least one regulatory sequence, and a cell, a plant, and a seed comprising the recombinant DNA construct. The cell may be eukaryotic, e.g., a yeast, insect, or plant cell, or prokaryotic, e.g., a bacterial cell.

### BRIEF DESCRIPTION OF THE FIGURES AND SEQUENCE LISTINGS

The invention can be more fully understood from the following detailed description and the accompanying drawings and Sequence Listing which form a part of this application.

FIG. 1 shows a map of the pHSbarENDs2 activation tagging construct (SEQ ID NO:1) used to make the Arabidopsis populations.

FIG. 2 shows a map of the vector pDONR™/Zeo (SEQ ID NO:2). The attP1 site is at nucleotides 570-801; the attP2 site is at nucleotides 2754-2985 (complementary strand).

FIG. 3 shows a map of the vector pDONR™221 (SEQ ID NO:3). The attP1 site is at nucleotides 570-801; the attP2 site is at nucleotides 2754-2985 (complementary strand).

FIG. 4 shows a map of the vector pBC-yellow (SEQ ID NO:4), a destination vector for use in construction of expression vectors for Arabidopsis. The attR1 site is at nucleotides 11276-11399 (complementary strand); the attR2 site is at nucleotides 9695-9819 (complementary strand).

FIG. 5 shows a map of PHP27840 (SEQ ID NO:5), a destination vector for use in construction of expression vectors for soybean. The attR1 site is at nucleotides 7310-7434; the attR2 site is at nucleotides 8890-9014.

FIG. 6 shows a map of PHP23236 (SEQ ID NO:6), a destination vector for use in construction of expression vectors for Gaspe Flint derived maize lines. The attR1 site is at nucleotides 2006-2130; the attR2 site is at nucleotides 2899-3023.

FIG. 7 shows a map of PHP10523 (SEQ ID NO:7), a plasmid DNA present in Agrobacterium strain LBA4404.

FIG. 8 shows a map of PHP23235 (SEQ ID NO:8), a vector used to construct the destination vector PHP23236.

FIG. 9 shows a map of the entry clone PHP20234 (SEQ ID NO:9), a vector carrying the PINII terminator. The attR2 site is at nucleotides 591-747; the attL3 site is at nucleotides 1100-1195.

FIG. 10 shows a map of PHP28529 (SEQ ID NO:10), a destination vector for use in construction of expression vectors for maize lines. The attR3 site is at nucleotides 3613-3737; the attR4 site is at nucleotides 2035-2159.

FIG. 11 shows a map of the entry clone PHP28408 (SEQ ID NO:11), a vector carrying the constitutive maize GOS2 promoter. The attL4 site is at nucleotides 160-255; the attR1 site is at nucleotides 2301-2447.

FIG. 12 shows a map of the entry clone PHP22020 (SEQ ID NO:12), a vector carrying the root maize NAS2 promoter. The attR1 site is at nucleotides 31-187; the attL4 site is at nucleotides 2578-2673.

FIG. 13 shows a map of PHP29635 (SEQ ID NO:13), a destination vector for use in construction of expression vectors for Gaspe Flint derived maize lines. The attR1 site is at nucleotides 40786-40910; the attR2 site is at nucleotides 41679-41803.

FIG. 14 shows a map of PIIOXS2a-FRT87(ni)m (SEQ ID NO:18), a vector used to construct the destination vector PHP29635.

FIGS. 15A through 15K show the multiple alignment of the full length amino acid sequences of SEQ ID NOs: 27, 29, 35, 37, 39, 41, 43, 46, 47, 48, 49, 50, 51, 52, 53, 54, 55, 56, 57, 58, 59, 68 or 70. Residues that match the Consensus sequence exactly are shaded. The consensus sequence is shown above each alignment. The consensus residues are determined by a straight majority.

FIG. 16 shows a chart of the percent sequence identity and the divergence values for each pair of amino acid sequences of the LPK homologs displayed in FIGS. 15A through 15K.

FIG. 17 is the growth medium used for semi-hydroponics maize growth in Example 17.

FIG. 18 is a chart setting forth data relating to the effect of different nitrate concentrations on the growth and development of Gaspe Flint derived maize lines in Example 17.

FIG. 19 shows a map of PHP28647 (SEQ ID NO:62).

FIG. 20 shows a map of PHP33692 (SEQ ID NO:63).

FIG. 21 shows a map of PHP19770 (SEQ ID NO:64).

FIG. 22 shows a map of PHP21737 (SEQ ID NO:65).

FIG. 23 shows a map of PHP29559 (SEQ ID NO:66)

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

SEQ ID NO:1 pHSbarENDs2

SEQ ID NO:2 pDONR™/Zeo

SEQ ID NO:3 pDONR™221

SEQ ID NO:4 pBC-yellow

SEQ ID NO:5 PHP27840

SEQ ID NO:6 PHP23236

SEQ ID NO:7 PHP10523

SEQ ID NO:8 PHP23235

SEQ ID NO:9 PHP20234

SEQ ID NO:10 PHP28529

SEQ ID NO:11 PHP28408

SEQ ID NO:12 PHP22020

SEQ ID NO:13 PHP29635

SEQ ID NO:14 is the attB1 sequence.

SEQ ID NO:15 is the attB2 sequence.

SEQ ID NO:16 is the forward primer VC062 in Example 9.

SEQ ID NO:17 is the reverse primer VC063 in Example 9.

SEQ ID NO:18 PIIOXS2a-FRT87(ni)m.

SEQ ID NO:19 is the maize NAS2 promoter.

SEQ ID NO:20 is the GOS2 promoter.

SEQ ID NO:21 is the ubiquitin promoter.

SEQ ID NO:22 is the S2A promoter.

SEQ ID NO:23 is the PINII terminator.

TABLE 1
LPK domain-containing protein (LPK)

| | | SEQ ID NO: | SEQ ID NO: |
|---|---|---|---|
| Protein | Clone Designation | (Nucleotide) | (Amino Acid) |
| LPK | p0050.cjlae47r | 24 | 25 |
| LPK | p0110.cgsmp02r | 26 | 27 |
| LPK | cfp6n.pk065.d16:fis | 28 | 29 |
| LPK | sbacm.pk094.p8.f | 30 | 31 |
| LPK | adf2c.pk002.h2 | 32 | 33 |
| LPK | Pred1* | 34 | 35 |
| LPK | Pred2* | 36 | 37 |
| LPK | Pred3* | 38 | 39 |
| LPK | custom6.pk880.d9** | 40 | 41 |
| LPK | custom5.pk735.d4 | 42 | 43 |
| LPK | Resurrection grass | 67 | 68 |
| | assm_NODE_38031 | | |
| LPK | Resurrection grass | 69 | 70 |
| | assm_NODE_175180 | | |

*Sequences labeled with a star were derived from partial sequence clones using sequence prediction software. Pred1 was derived using FGENESH prediction (see also Example 8) from public BAC AC216209 ZMMBBc0221J05 chr1 and the sequence of clone p0050.cjlae47r. Pred2 was derived using public gene prediction Glyma18g40290.1 JGI version 1.01 and the sequence of clone sbacm.pk094.p8.f. Pred3 was derived using public gene prediction At5g60280.1 TAIR version 9 and the sequence of clone adf2c.pk002.h2.. Additionally, these sequences were aligned with sequences from other species, and manually edited to remove putative introns. Primers designed based on the genomic locus of each of the sequences were used for long range genomic PCR capture. The resulting PCR product was sequenced and the FGENESH program and manually editing was used to predict each coding sequences.
**this sequence was captured using public BAC b0245n20 published in the Maize GDB and primers LPK F and LPK R (SEQ ID NO: 60 and SEQ ID NO: 61, respectively).

SEQ ID NO:44 corresponds to the nucleotide sequence (locus AT5G60270) encoding an Arabidopsis thaliana Lectin protein Kinase (LPK) protein.

SEQ ID NO:45 corresponds to the coding sequence of the Arabidopsis thaliana LPK encoded by nucleotides 191-1081 (Stop) of SEQ ID NO:34.

SEQ ID NO:46 corresponds to the Arabidopsis thaliana LPK domain-containing protein (LPK) (AT5G60270) locus.

SEQ ID NO:47 corresponds to NCBI GI NO:226528693 (Zea mays).

SEQ ID NO:48 corresponds to NCBI GI NO:226502714 (Zea mays).

SEQ ID NO:49 corresponds to NCBI GI NO:242032451 (Sorghum bicolor).

SEQ ID NO:50 corresponds to NCBI GI NO:38112427 (Medicago trunculata)

SEQ ID NO:51 corresponds to NCBI GI NO:15239261 (Arabidopsis thaliana).

SEQ ID NO:52 corresponds to NCBI GI NO:242095594 (Sorghum bicolor).

SEQ ID NO:53 corresponds to NCBI GI NO:15239260 (Arabidopsis thaliana).

SEQ ID NO:54 corresponds to SEQ ID NO:138302 in EP2090662 (Oryza sativa).

SEQ ID NO:55 corresponds to SEQ ID NO:69317 in EP2090662 (Zea mays).

SEQ ID NO:56 corresponds to SEQ ID NO:193726 in EP2090662 (Glycine max).

SEQ ID NO:57 corresponds to SEQ ID NO: 167274 in EP 2090662 (Arabidopsis thaliana).

SEQ ID NO:58 corresponds to SEQ ID NO:167322 in EP2090662 (Zea mays).

SEQ ID NO:59 corresponds to SEQ ID NO:167272 in EP2090662 (Arabidopsis thaliana).

SEQ ID NO:60 corresponds to the nucleotide sequence of the LPK F primer.

SEQ 1D NO:61 corresponds to the nucleotide sequence of the LPK R primer.

SEQ ID NO:62 corresponds to the nucleotide sequence vector of PHP28647.

SEQ ID NO:63 corresponds to the nucleotide sequence of vector PHP33692.

SEQ ID NO:64 corresponds to the nucleotide sequence of vector PHP19770.

SEQ ID NO:65 corresponds to the nucleotide sequence of vector PHP21737.

SEQ ID NO:66 corresponds to the nucleotide sequence of vector PHP29559.

SEQ ID NO:67 corresponds to the nucleotide sequence of Resurrection grass node 38031.

SEQ ID NO:68 corresponds to the protein sequence of Resurrection grass node 38031.

SEQ ID NO:69 corresponds to the nucleotide sequence of Resurrection grass node 175180.

SEQ ID NO:70 corresponds to the nucleotide sequence of Resurrection grass node 175180.

SEQ ID NO:71 corresponds to the protein sequence of the hypothetical protein from Sorghum bicolor, XP002461846.

SEQ ID NO:72 corresponds to the protein sequence of the hypothetical protein from Sorghum bicolor, XP002441536.

### DETAILED DESCRIPTION OF EMBODIMENTS

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

The term “root architecture” refers to the arrangement of the different parts that comprise the root. The terms “root architecture”, “root structure”, “root system” or “root system architecture” are used interchangeably herewithin.

In general, the first root of a plant that develops from the embryo is called the primary root. In most dicots, the primary root is called the taproot. This main root grows downward and gives rise to branch (lateral) roots. In monocots the primary root of the plant branches, giving rise to a fibrous root system.

The term “altered root architecture” refers to aspects of alterations of the different parts that make up the root system at different stages of its development compared to a reference or control plant. It is understood that altered root architecture encompasses alterations in one or more measurable parameters, including but not limited to, the diameter, length, number, angle or surface of one or more of the root system parts, including but not limited to, the primary root, lateral or branch root, adventitious root, and root hairs, all of which fall within the scope of this invention. These changes can lead to an overall alteration in the area or volume occupied by the root. The reference or control plant does not comprise in its genome the recombinant DNA construct or heterologous construct.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

The term “V” stage refers to the leaf stages of a corn plant; e.g. V4=four, V5=five leaves with visible leaf collars. The leaf collar is the light-colored collar-like “band” located at the base of an exposed leaf blade, near the spot where the leaf blade comes in contact with the stem of the plant. The leaves are counted beginning with the lowermost, short, rounded-tip true leaf and ending with the uppermost leaf with a visible leaf collar.

“Agronomic characteristics” is a measurable parameter including but not limited to of greenness, yield, growth rate, biomass, fresh weight at maturation, dry weight at maturation, fruit yield, seed yield, total plant nitrogen content, fruit nitrogen content, seed nitrogen content, whole plant free amino acid content, fruit free amino acid content, seed free amino acid content, fruit protein content, seed protein content, protein content in a vegetative tissue, drought tolerance, nitrogen uptake, root lodging, harvest index, stalk lodging, plant height, ear height, ear length, early seedling vigor and seedling emergence under low temperature stress.

“Ipk” (lectin protein kinase), “at-lpk” (Arabidopsis thaliana—lectin protein kinase) are used interchangeably herewithin and refer to the Arabidopsis thaliana locus, AT5G60270 (SEQ ID NO:34) and to nucleotide homologs from different species, such as corn, of the Arabidopsis thaliana “LPK” locus, AT5G60270 and includes without limitation the nucleotide sequences of SEQ ID NOs:24, 26, 28, 30, 32, 67 and 69. In addition two different splice variants of the Arabidopsis lpk gene have been identified: AT5G60270 (SEQ ID NO: 36) and AT5G60270 (SEQ ID NO:38), both encode the amino acid sequence presented in SEQ ID NO: 41.

“LPK”, “AT-LPK”, lectin protein kinase refers to the protein (SEQ ID NO:40) encoded by AT5G60270 (SEQ ID NO:34) and to protein homologs from different species, such as corn, soybean, and Arabidopsis thaliana, of the Arabidopsis thaliana “LPK” and includes without limitation the amino acid sequence of SEQ ID NOs: 27, 29, 35, 37, 39, 41, 43, 46, 47, 48, 49, 50, 51, 52, 53, 54, 55, 56, 57, 58, 59, 68 or 70.

Environmental conditions” refer to conditions under which the plant is grown, such as the availability of water, availability of nutrients (for example nitrogen), or the presence of disease.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Transgenic plant” includes reference to a plant which comprises within its genome a heterologous polynucleotide. The heterologous polynucleotide may be stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Association mapping of genes. The goal of gene mapping is to identify genes which contribute to phenotypes of interest. The first stage of mapping is usually to locate a general region of a chromosome which is associated with transmission of the phenotypes of interest of interest. Next, the gene and ultimately, particular alleles, are identified as having a causative role.

One approach to gene mapping (linkage analysis) uses maize lines with a known pedigree structure. Individuals are genotyped at random markers spread across the genome. If a disease gene is close to one of the markers then, within the pedigree, the inheritance pattern at the marker will mimic the inheritance pattern of the phenotype of interest. Linkage analysis has been highly successful at finding genes for simple genes associated with a phenotype of interest: i.e., those in which a single major gene is responsible for the photype in a given pedigree, and environmental factors are not very important.

A second approach to gene mapping (association, or disequilibrium mapping) uses associations at the population level. The idea is that a phenotype of interest arises on a particular haplotype background, and so individuals who inherit the phenotype of interest will also inherit the same alleles at nearby marker loci. This process is complicated by recombination and mutation. In a sense, association mapping is not fundamentally different from linkage analysis, but instead of using a family pedigree, an unknown population genealogy is used. Because the population genealogy is much deeper than a family pedigree, disequilibrium mapping permits much finer-scale mapping than does linkage analysis. An overview of the association mapping strategies and analysis is given in “Association Mapping in Plants, Oraguzie, N. C.; Rikkerink, E. H. A.; Gardiner, S. E.; Silva, H. N.d. Springer January 2007.”

Turning now to embodiments:

Embodiments include isolated polynucleotides and polypeptides, recombinant DNA constructs, compositions (such as plants or seeds) comprising these recombinant DNA constructs, and methods utilizing these recombinant DNA constructs.

Isolated Polynucleotides and Polypeptides

The present invention includes the following isolated polynucleotides and polypeptides:

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70; or (ii) a full complement of the nucleic acid sequence of (i). Any of the foregoing isolated polynucleotides may be utilized in any recombinant DNA constructs (including suppression DNA constructs) of the present invention. The polypeptide may be a LPK protein.

An isolated polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70. The polypeptide may be a LPK protein.

An isolated polynucleotide comprising (i) a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 26, 28, 34, 36, 38, 40, 42, 45, 67 or 69 (ii) a full complement of the nucleic acid sequence of (i). Any of the foregoing isolated polynucleotides may be utilized in any recombinant DNA constructs (including suppression DNA constructs) of the present invention. The isolated polynucleotide encodes a LPK protein.

Recombinant DNA Constructs and Suppression DNA Constructs.

In one aspect, the present invention includes recombinant DNA constructs (including suppression DNA constructs).

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 26, 28, 34, 36, 38, 40, 42, 45, 67 or 69 (ii) a full complement of the nucleic acid sequence of (i).

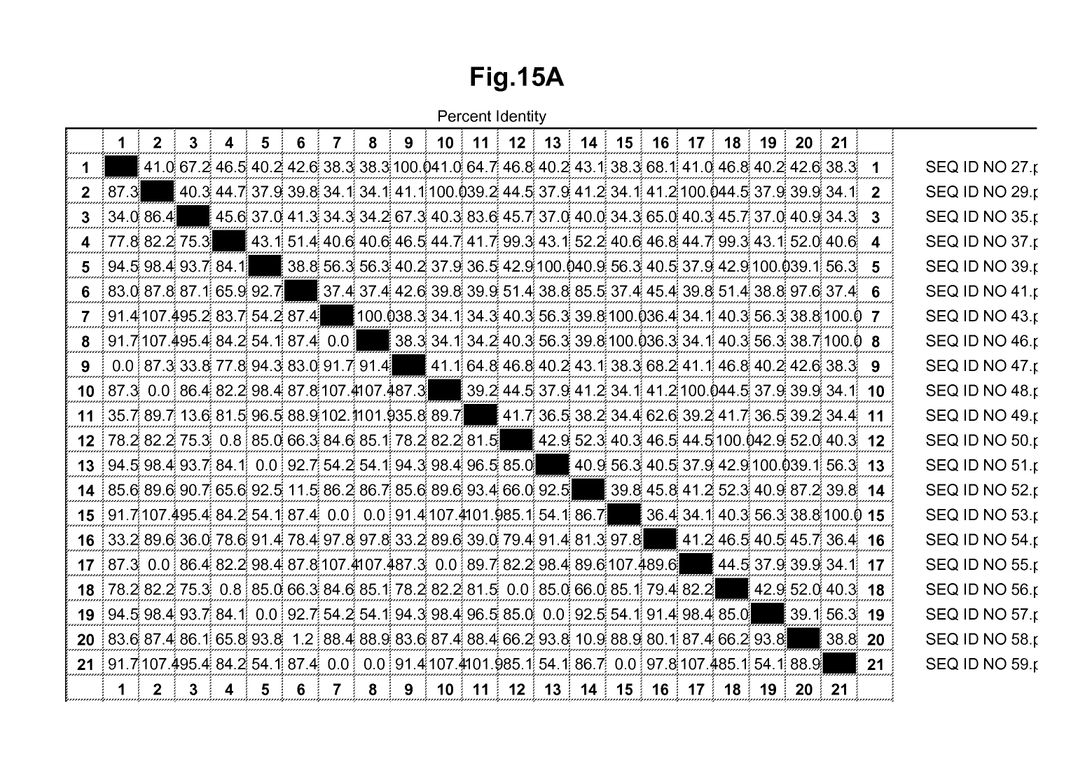

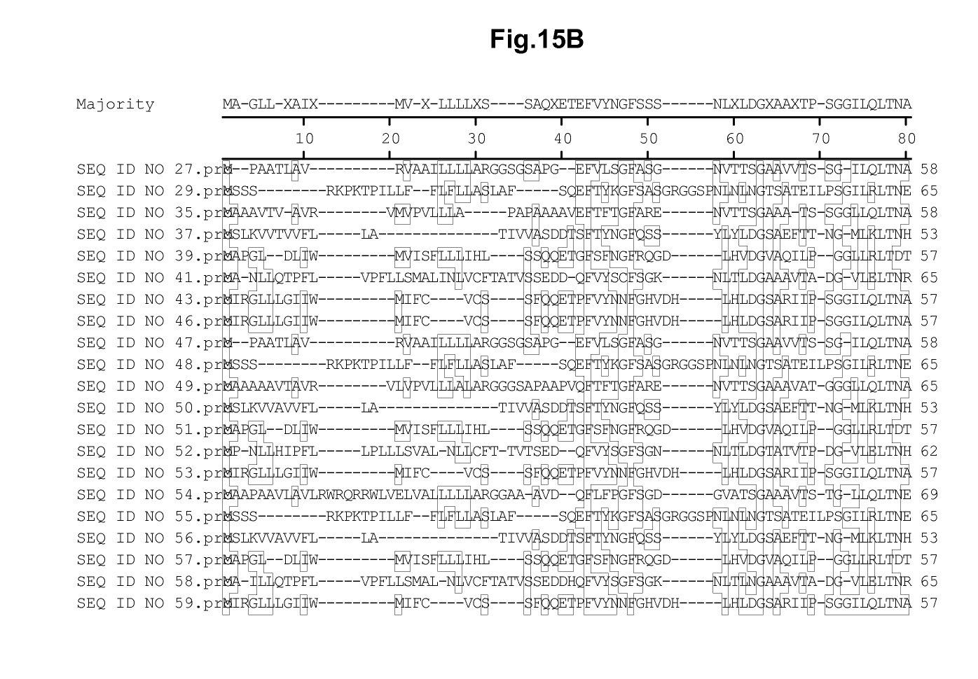

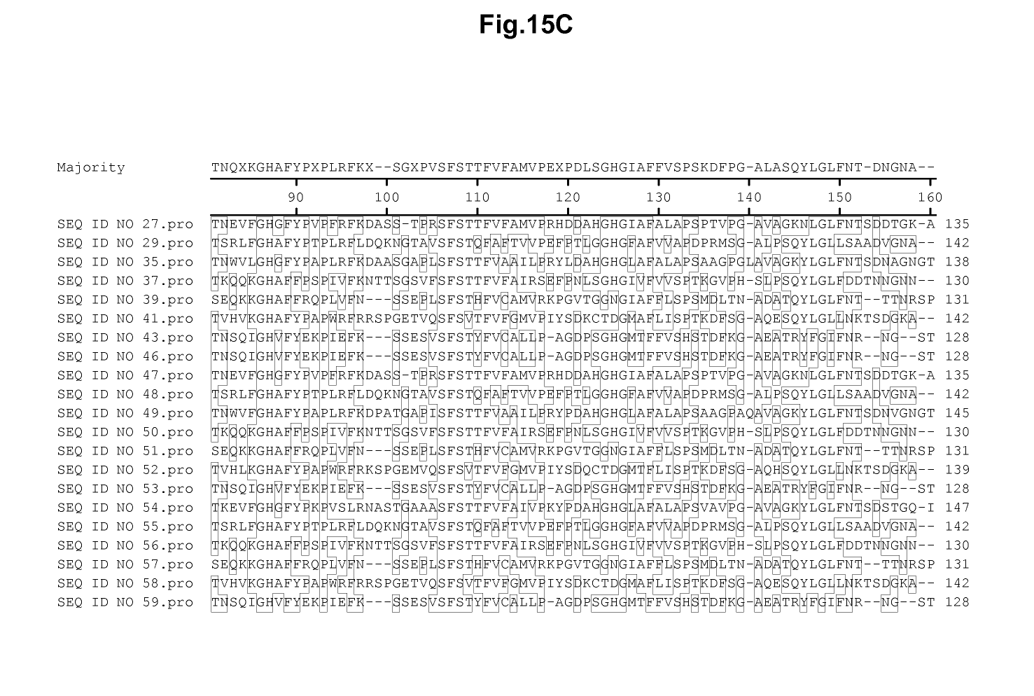

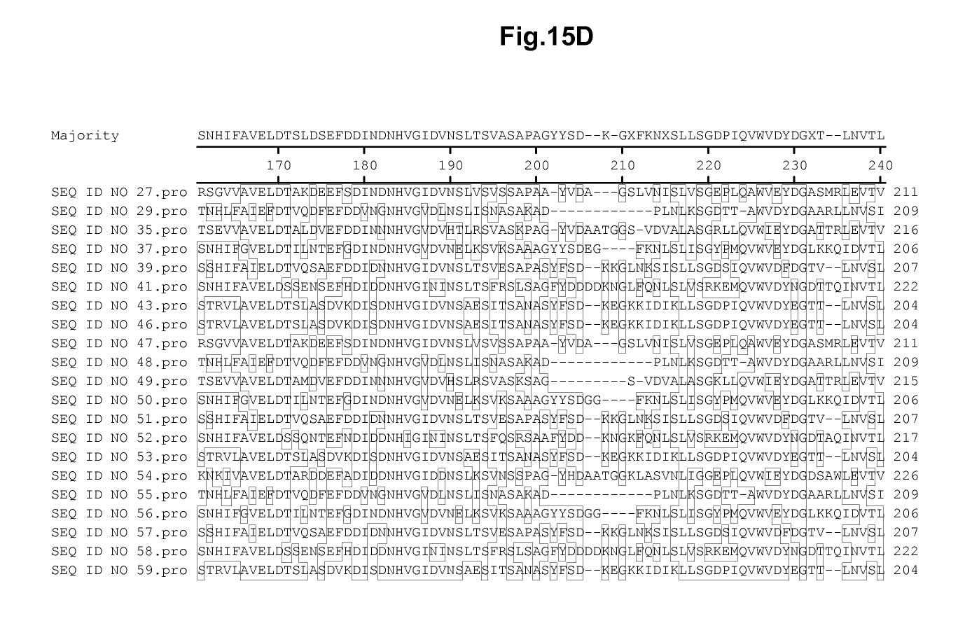

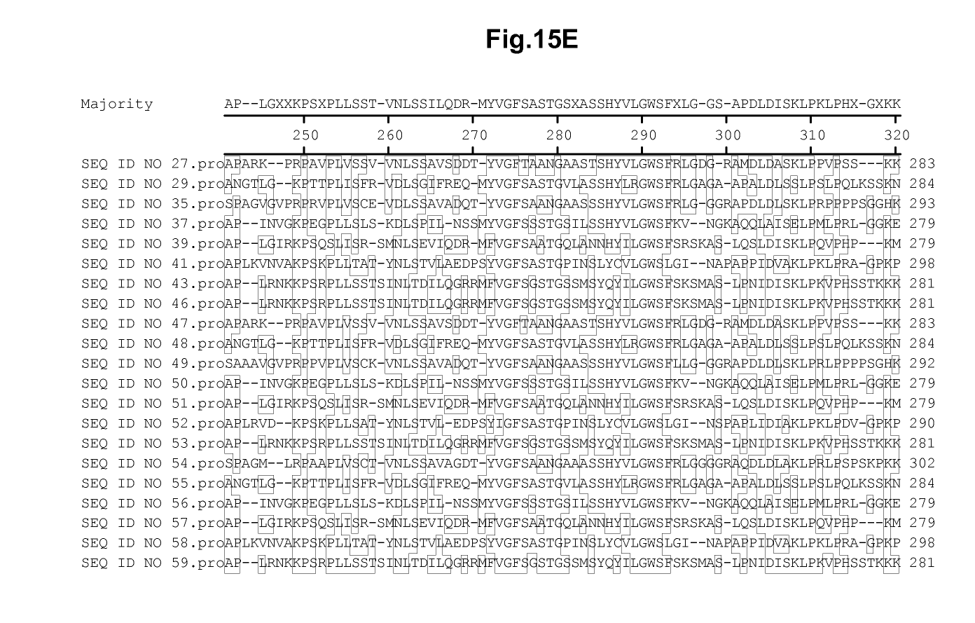

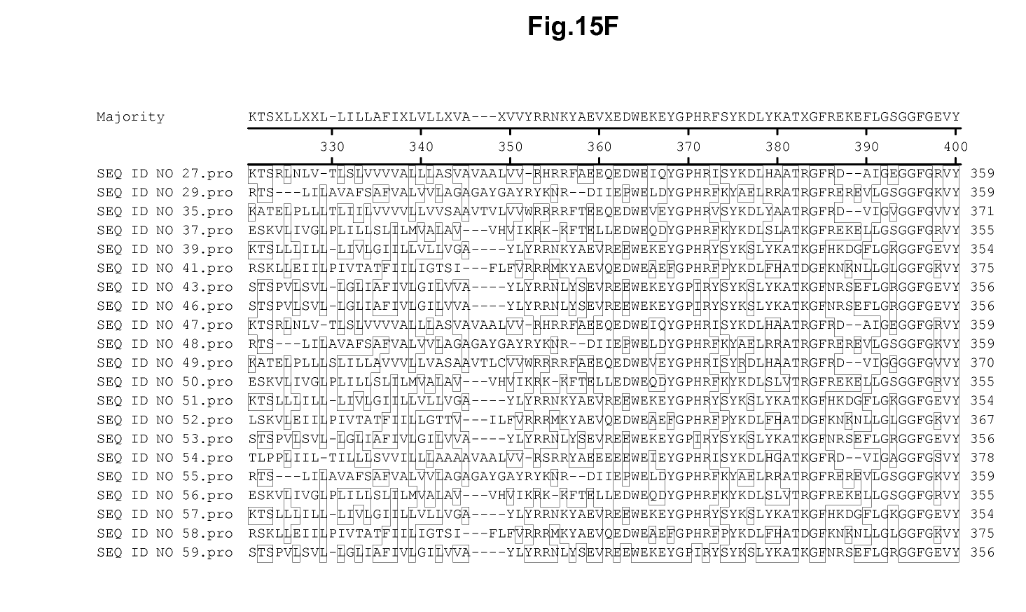

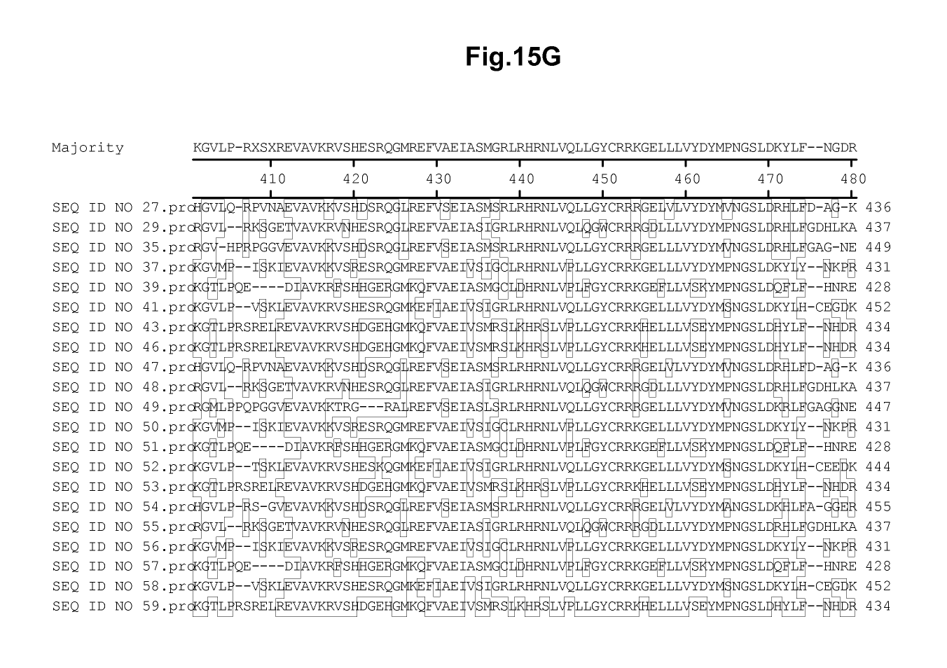

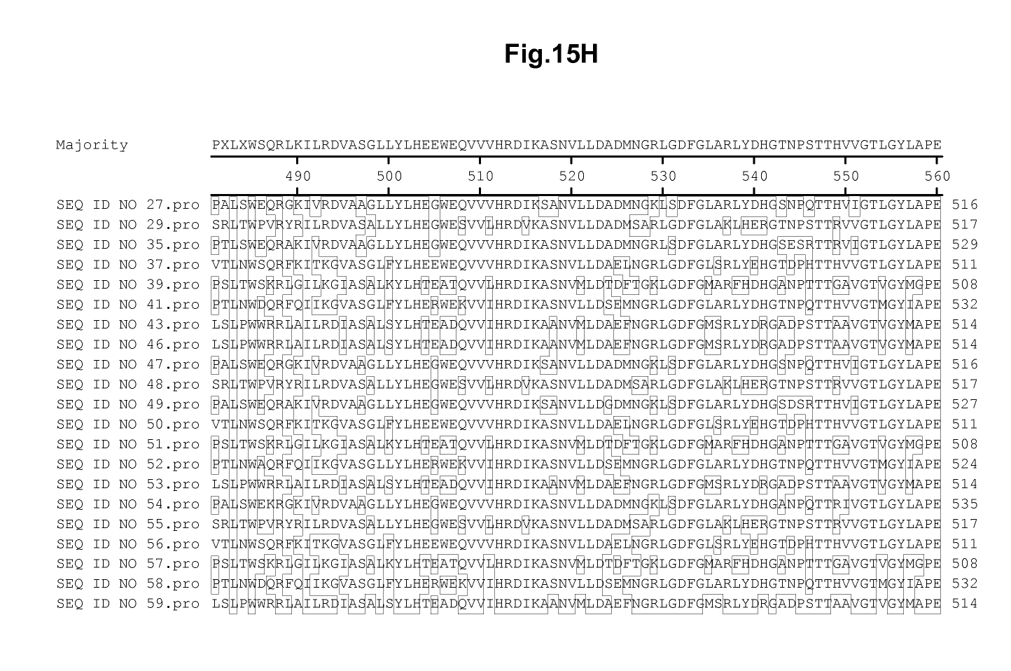

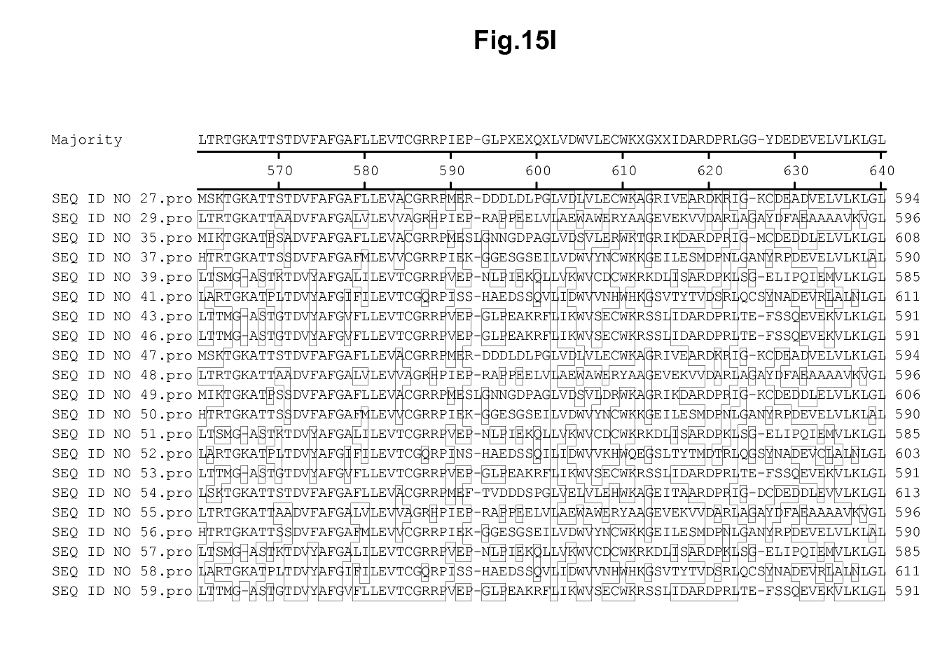

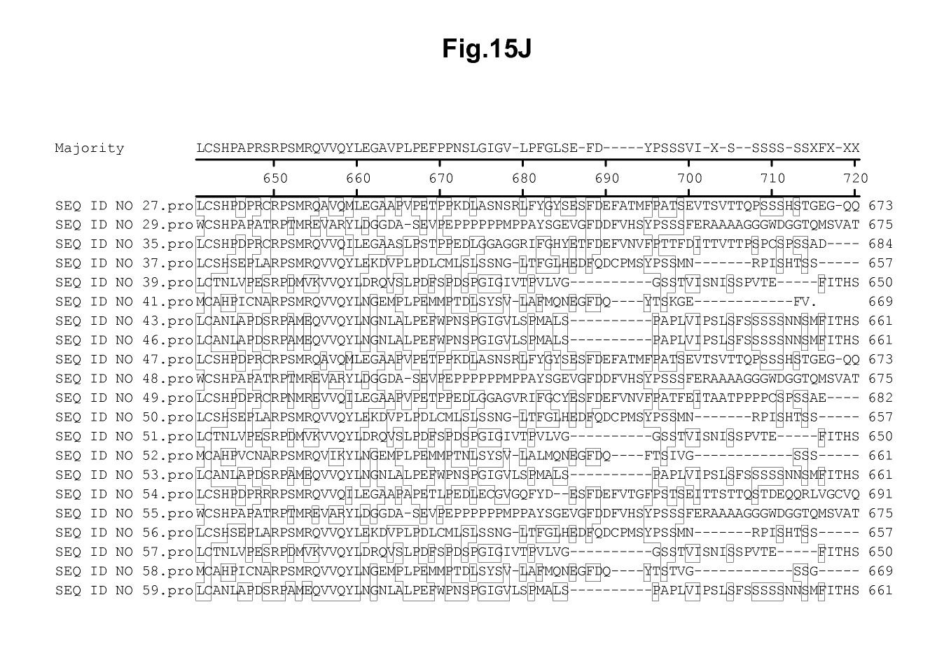

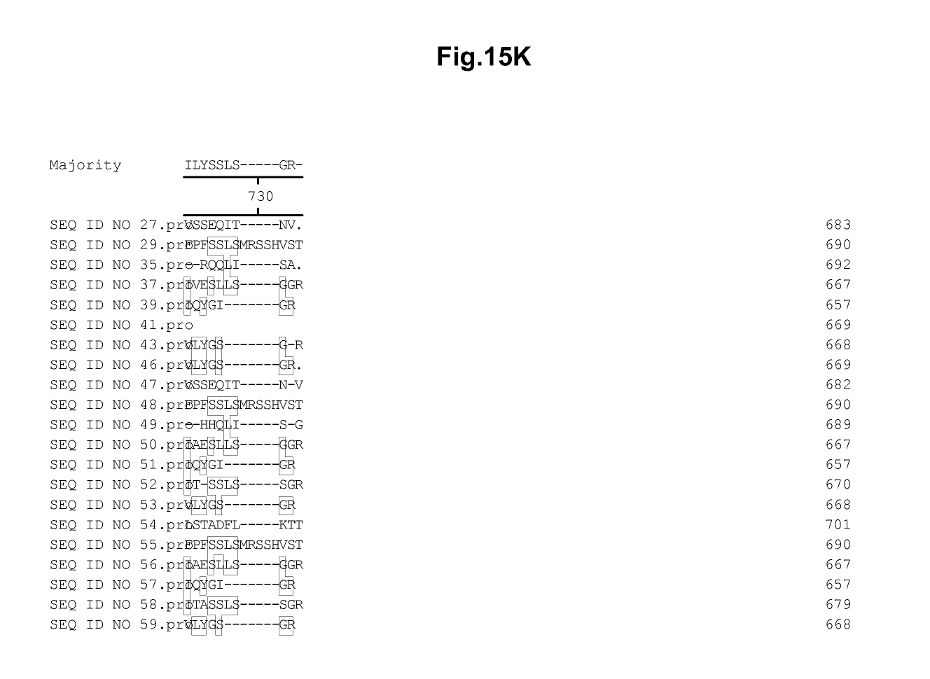

FIGS. 15A through 15 K shows the multiple alignment of the full length amino acid sequences of SEQ ID NOs: 27, 29, 35, 37, 39, 41, 43, 46, 47, 48, 49, 50, 51, 52, 53, 54, 55, 56, 57, 58, and 59. The multiple alignment of the sequences was performed using the Megalign® program of the LASERGENE® bioinformatics computing suite (DNASTAR® Inc., Madison, Wis.); in particular, using the Clustal V method of alignment (Higgins and Sharp (1989) CABIOS. 5:151-153) with the multiple alignment default parameters of GAP PENALTY=10 and GAP LENGTH PENALTY=10, and the pairwise alignment default parameters of KTUPLE=1, GAP PENALTY=3, WINDOW=5 and DIAGONALS SAVED=5.

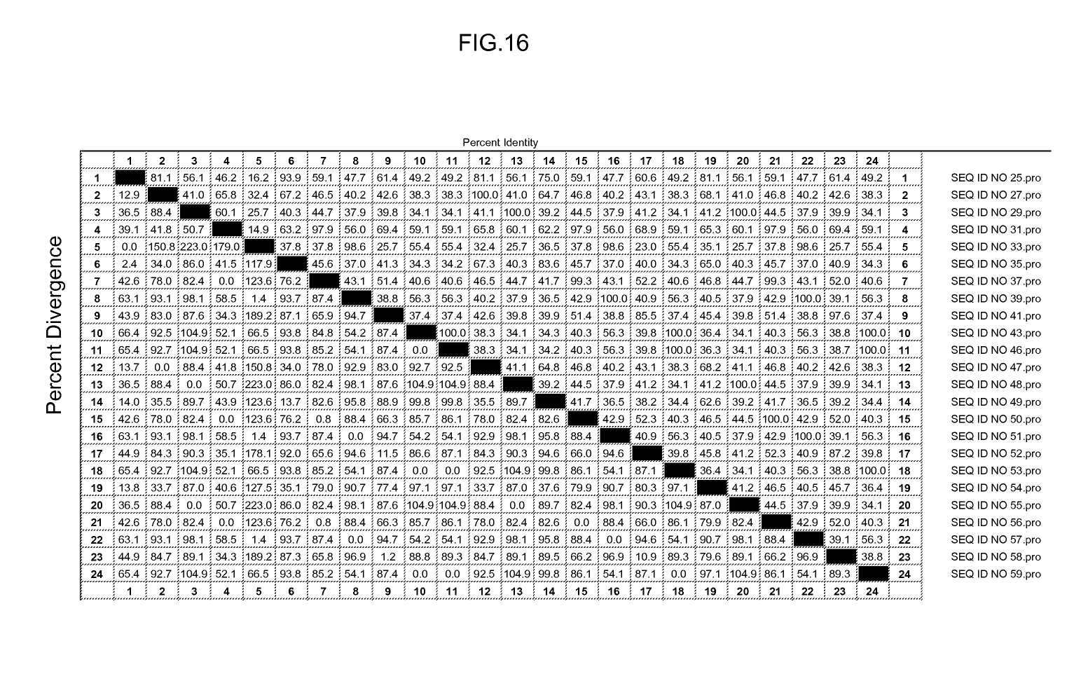

FIG. 16 shows the percent sequence identity and the divergence values for each pair of amino acids sequences displayed in FIGS. 15A through 15K.

In another embodiment, a recombinant DNA construct comprises a polynucleotide operably linked to at least one regulatory sequence (e.g., a promoter functional in a plant), wherein said polynucleotide encodes a LPK protein.

In another aspect, the present invention includes suppression DNA constructs.

A suppression DNA construct may comprise at least one regulatory sequence (e.g. a promoter functional in a plant) operably linked to (a) all or part of (i) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a LPK protein; or (c) all or part of (i) a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 26, 28, 34, 36, 38, 40, 42, 45, 67, or 69 (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The term “suppression” includes lower, reduce, decline, decrease, inhibit, eliminate or prevent. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as sRNA (short interfering RNA) constructs and miRNA (microRNA) constructs.

“Antisense inhibition” refers to the production of antisense RNA transcripts capable of suppressing the expression of the target protein.

“Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target protein. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al. (1998) Plant J. 16:651-659; and Gura (2000) Nature 404:804-808).

Another variation describes the use of plant viral sequences to direct the suppression of proximal mRNA encoding sequences (PCT Publication WO 98/36083 published on Aug. 20, 1998).

Previously described is the use of “hairpin” structures that incorporate all, or part, of an mRNA encoding sequence in a complementary orientation that results in a potential “stem-loop” structure for the expressed RNA (PCT Publication WO 99/53050 published on Oct. 21, 1999). In this case the stem is formed by polynucleotides corresponding to the gene of interest inserted in either sense or anti-sense orientation with respect to the promoter and the loop is formed by some polynucleotides of the gene of interest, which do not have a complement in the construct. This increases the frequency of cosuppression or silencing in the recovered transgenic plants. For review of hairpin suppression see Wesley, S. V. et al. (2003) Methods in Molecular Biology, Plant Functional Genomics: Methods and Protocols 236:273-286.

A construct where the stem is formed by at least 30 nucleotides from a gene to be suppressed and the loop is formed by a random nucleotide sequence has also effectively been used for suppression (PCT Publication No. WO 99/61632 published on Dec. 2, 1999).

The use of poly-T and poly-A sequences to generate the stem in the stem-loop structure has also been described (PCT Publication No. WO 02/00894 published Jan. 3, 2002).

Yet another variation includes using synthetic repeats to promote formation of a stem in the stem-loop structure. Transgenic organisms prepared with such recombinant DNA fragments have been shown to have reduced levels of the protein encoded by the nucleotide fragment forming the loop as described in PCT Publication No. WO 02/00904, published 3 Jan. 2002.

RNA interference refers to the process of sequence-specific post-transcriptional gene silencing in animals mediated by short interfering RNAs (siRNAs) (Fire et al., Nature 391:806 1998). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 1999). Such protection from foreign gene expression may have evolved in response to the production of double-stranded RNAs (dsRNAs) derived from viral infection or from the random integration of transposon elements into a host genome via a cellular response that specifically destroys homologous single-stranded RNA of viral genomic RNA. The presence of dsRNA in cells triggers the RNAi response through a mechanism that has yet to be fully characterized.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein et al., Nature 409:363, 2001). Short interfering RNAs derived from dicer activity are typically about 21 to about 23 nucleotides in length and comprise about 19 base pair duplexes (Elbashir et al., Genes Dev. 15:188, 2001). Dicer has also been implicated in the excision of 21- and 22-nucleotide small temporal RNAs (stRNAs) from precursor RNA of conserved structure that are implicated in translational control (Hutvagner et al., Science 293:834, 2001). The RNAi response also features an endonuclease complex, commonly referred to as an RNA-induced silencing complex (RISC), which mediates cleavage of single-stranded RNA having sequence complementarity to the antisense strand of the sRNA duplex. Cleavage of the target RNA takes place in the middle of the region complementary to the antisense strand of the sRNA duplex (Elbashir et al., Genes Dev. 15:188, 2001). In addition, RNA interference can also involve small RNA (e.g., miRNA) mediated gene silencing, presumably through cellular mechanisms that regulate chromatin structure and thereby prevent transcription of target gene sequences (see, e.g., Allshire, Science 297:1818-1819, 2002; Volpe et al., Science 297:1833-1837, 2002; Jenuwein, Science 297:2215-2218, 2002; and Hall et al., Science 297:2232-2237, 2002). As such, miRNA molecules of the invention can be used to mediate gene silencing via interaction with RNA transcripts or alternately by interaction with particular gene sequences, wherein such interaction results in gene silencing either at the transcriptional or post-transcriptional level.

RNAi has been studied in a variety of systems. Fire et al. (Nature 391:806, 1998) were the first to observe RNAi in C. elegans. Wianny and Goetz (Nature Cell Biol. 2:70, 1999) describe RNAi mediated by dsRNA in mouse embryos. Hammond et al. (Nature 404:293, 2000) describe RNAi in Drosophila cells transfected with dsRNA. Elbashir et al., (Nature 411:494, 2001) describe RNAi induced by introduction of duplexes of synthetic 21-nucleotide RNAs in cultured mammalian cells including human embryonic kidney and HeLa cells.

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

It is thought that sequence complementarity between small RNAs and their RNA targets helps to determine which mechanism, RNA cleavage or translational inhibition, is employed. It is believed that siRNAs, which are perfectly complementary with their targets, work by RNA cleavage. Some miRNAs have perfect or near-perfect complementarity with their targets, and RNA cleavage has been demonstrated for at least a few of these miRNAs. Other miRNAs have several mismatches with their targets, and apparently inhibit their targets at the translational level. Again, without being held to a particular theory on the mechanism of action, a general rule is emerging that perfect or near-perfect complementarity causes RNA cleavage, whereas translational inhibition is favored when the miRNA/target duplex contains many mismatches. The apparent exception to this is microRNA 172 (miR172) in plants. One of the targets of miR172 is APETALA2 (AP2), and although miR172 shares near-perfect complementarity with AP2 it appears to cause translational inhibition of AP2 rather than RNA cleavage.

MicroRNAs (miRNAs) are noncoding RNAs of about 19 to about 24 nucleotides (nt) in length that have been identified in both animals and plants (Lagos-Quintana et al., Science 294:853-858 2001, Lagos-Quintana et al., Curr. Biol. 12:735-739, 2002; Lau et al., Science 294:858-862, 2001; Lee and Ambros, Science 294:862-864, 2001; Llave et al., Plant Cell 14:1605-1619, 2002; Mourelatos et al., Genes. Dev. 16:720-728, 2002; Park et al., Curr. Biol. 12:1484-1495, 2002; Reinhart et al., Genes. Dev. 16:1616-1626, 2002). They are processed from longer precursor transcripts that range in size from approximately 70 to 200 nt, and these precursor transcripts have the ability to form stable hairpin structures. In animals, the enzyme involved in processing miRNA precursors is called Dicer, an RNAse III-like protein (Grishok et al., Cell 106:23-34, 2001; Hutvagner et al., Science 293:834-838, 2001; Ketting et al., Genes. Dev. 15:2654-2659, 2001). Plants also have a Dicer-like enzyme, DCL1 (previously named CARPEL FACTORY/SHORT INTEGUMENTS1/SUSPENSOR1), and recent evidence indicates that it, like Dicer, is involved in processing the hairpin precursors to generate mature miRNAs (Park et al., Curr. Biol. 12:1484-1495, 2002; Reinhart et al., Genes. Dev. 16:1616-1626, 2002). Furthermore, it is becoming clear from recent work that at least some miRNA hairpin precursors originate as longer polyadenylated transcripts, and several different miRNAs and associated hairpins can be present in a single transcript (Lagos-Quintana et al., Science 294:853-858, 2001; Lee et al., EMBO J. 21:4663-4670, 2002). Recent work has also examined the selection of the miRNA strand from the dsRNA product arising from processing of the hairpin by DICER (Schwartz, et al., Cell 115:199-208, 2003). It appears that the stability (i.e. G:C vs. A:U content, and/or mismatches) of the two ends of the processed dsRNA affects the strand selection, with the low stability end being easier to unwind by a helicase activity. The 5′ end strand at the low stability end is incorporated into the RISC complex, while the other strand is degraded.

MicroRNAs appear to regulate target genes by binding to complementary sequences located in the transcripts produced by these genes. In the case of lin-4 and let-7, the target sites are located in the 3′ UTRs of the target mRNAs (Lee et al., Cell 75:843-854, 1993; Wightman et al., Cell 75:855-862, 1993; Reinhart et al., Nature 403:901-906, 2000; Slack et al., Mol. Cell. 5:659-669, 2000), and there are several mismatches between the lin-4 and let-7 miRNAs and their target sites. Binding of the lin-4 or let-7 miRNA appears to cause downregulation of steady-state levels of the protein encoded by the target mRNA without affecting the transcript itself (Olsen and Ambros, Dev. Biol. 216:671-680, 1999). On the other hand, recent evidence suggests that miRNAs can in some cases cause specific RNA cleavage of the target transcript within the target site, and this cleavage step appears to require 100% complementarity between the miRNA and the target transcript (Hutvagner and Zamore, Science 297:2056-2060, 2002; Llave et al., Plant Cell 14:1605-1619, 2002). It seems likely that miRNAs can enter at least two pathways of target gene regulation: Protein downregulation when target complementarity is <100%, and RNA cleavage when target complementarity is 100%. MicroRNAs entering the RNA cleavage pathway are analogous to the 21-25 nt short interfering RNAs (siRNAs) generated during RNA interference (RNAi) in animals and posttranscriptional gene silencing (PTGS) in plants (Hamilton and Baulcombe 1999; Hammond et al., 2000; Zamore et al., 2000; Elbashir et al., 2001), and likely are incorporated into an RNA-induced silencing complex (RISC) that is similar or identical to that seen for RNAi.

Identifying the targets of miRNAs with bioinformatics has not been successful in animals, and this is probably due to the fact that animal miRNAs have a low degree of complementarity with their targets. On the other hand, bioinformatic approaches have been successfully used to predict targets for plant miRNAs (Llave et al., Plant Cell 14:1605-1619 2002; Park et al., Curr. Biol. 12:1484-1495 2002; Rhoades et al., Cell 110:513-520 2002), and thus it appears that plant miRNAs have higher overall complementarity with their putative targets than do animal miRNAs. Most of these predicted target transcripts of plant miRNAs encode members of transcription factor families implicated in plant developmental patterning or cell differentiation.

A recombinant DNA construct (including a suppression DNA construct) of the present invention may comprise at least one regulatory sequence.

A regulatory sequence is a promoter.

A number of promoters can be used in recombinant DNA constructs (and suppression DNA constructs) of the present invention. The promoters can be selected based on the desired outcome, and may include constitutive, tissue-specific, cell specific, inducible, or other promoters for expression in the host organism.

High level, constitutive expression of the candidate gene under control of the 35S or UBI promoter may have pleiotropic effects, although Candidate gene efficacy may be estimated when driven by a constitutive promoter.

Use of tissue-specific and/or stress-specific expression may eliminate undesirable effects but retain the ability to alter root architecture. This effect has been observed in Arabidopsis (Kasuga et al. (1999) Nature Biotechnol. 17:287-291). Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (UBI) (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the maize GOS2 promoter (WO0020571 A2, published Apr. 1, 2000) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the invention, it may be desirable to use a tissue-specific or developmentally regulated promoter.

A preferred tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present invention which causes the desired temporal and spatial expression.

Promoters which are seed or embryo specific and may be useful in the invention include soybean Kunitz trysin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J. 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters that may be used in the invention include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(½):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993)). “Structural characterization, chromosomal localization and phylogenetic evaluation of two pairs of AGAMOUS-like MADS-box genes from maize”, Theissen et al., Gene 156(2): 155-166 (1995); NCBI GenBank Accession No. X80206)). Zag2 transcripts can be detected 5 days prior to pollination to 7 to 8 days after pollination (DAP), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present invention in plants are vascular element specific or stalk-preferred promoters. Such stalk-preferred promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”. New promoters of various types useful in plant cells are constantly being discovered; numerous examples may be found in the compilation by Okamuro, J. K., and Goldberg, R. B., Biochemistry of Plants 15:1-82 (1989). (Put this with the other constitutive promoter description.)

Promoters that may be used in the invention include: RIP2, mLIP15, ZmCOR1, Rab17, CaMV 35S, RD29A, B22E, Zag2, SAM synthetase, ubiquitin (SEQ ID NO:21), CaMV 19S, nos, Adh, sucrose synthase, R-allele, root cell promoter, the vascular tissue specific promoters S2A (Genbank accession number EF030816; SEQ ID NO:22) and S2B (Genbank accession number EF030817) and the constitutive promoter GOS2 (SEQ ID NO:20) from Zea mays. Other promoters include root preferred promoters, such as the maize NAS2 promoter (SEQ ID NO:19), the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790, gi: 1063664).

A “substantial portion” of a nucleotide sequence comprises a nucleotide sequence that is sufficient to afford putative identification of the promoter that the nucleotide sequence comprises. Nucleotide sequences can be evaluated either manually, by one skilled in the art, or using computer-based sequence comparison and identification tools that employ algorithms such as BLAST (Basic Local Alignment Search Tool; Altschul et al. (1993) J. Mol. Biol. 215:403-410). In general, a sequence of thirty or more contiguous nucleotides is necessary in order to putatively identify a promoter nucleic acid sequence as homologous to a known promoter. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

Recombinant DNA constructs (and suppression DNA constructs) of the present invention may also include other regulatory sequences, including but not limited to, translation leader sequences, introns, and polyadenylation recognition sequences. In another embodiment of the present invention, a recombinant DNA construct of the present invention further comprises an enhancer or silencer.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell. Biol. 8:4395-4405 (1988); Callis et al., Genes Dev. 1:1183-1200 (1987). Such intron enhancement of gene expression is typically greatest when placed near the 5′ end of the transcription unit. Use of maize introns Adh1-S intron 1, 2, and 6, the Bronze-1 intron are known in the art. See generally, The Maize Handbook, Chapter 116, Freeling and Walbot, Eds., Springer, N.Y. (1994).

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or from any other eukaryotic gene.

A translation leader sequence is a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. Molecular Biotechnology 3:225 (1995)).

In another embodiment of the present invention, a recombinant DNA construct of the present invention further comprises an enhancer or silencer.

Any plant can be selected for the identification of regulatory sequences and genes to be used in creating recombinant DNA constructs and suppression DNA constructs of the present invention. Examples of suitable plant targets for the isolation of genes and regulatory sequences would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassaya, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, a vine, watermelon, wheat, yams, and zucchini. Plants for the identification of regulatory sequences also include Arabidopsis, corn, wheat, soybean, and cotton.

### Compositions

A composition of the present invention is a plant comprising in its genome any of the recombinant DNA constructs (including any of the suppression DNA constructs) of the present invention (such as the constructs discussed above). Additional compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

Preferably, in hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit altered root (or plant) architecture, or used in a breeding program to produce hybrid seed, which can be grown to produce plants that would exhibit altered root (or plant) architecture. The seeds may be maize.

The plant may be a monocotyledonous or dicotyledonous plant, such as a maize or soybean plant, or a maize plant, such as a maize hybrid plant or a maize inbred plant. The plant may also be sunflower, sorghum, castor bean, grape, canola, wheat, alfalfa, cotton, rice, barley or millet.

The recombinant DNA construct may be stably integrated into the genome of the plant.

Other embodiments include but are not limited to the following preferred embodiments:

1. A plant (for example, a maize or soybean plant) comprising in its genome a recombinant DNA construct comprising a polynucleotide operably linked to at least one regulatory sequence, wherein said polynucleotide encodes a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, and wherein said plant exhibits an altered root architecture when compared to a control plant not comprising said recombinant DNA construct. The plant may further exhibit an alteration of at least one agronomic characteristic when compared to the control plant.

2. A plant (for example, a maize or soybean plant) comprising in its genome:

a recombinant DNA construct comprising:

(a) a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or

(b) a suppression DNA construct comprising at least one regulatory element operably linked to:
(i) all or part of: (A) a nucleic acid sequence encoding a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (B) a full complement of the nucleic acid sequence of (b)(i)(A); or
(ii) a region derived from all or part of a sense strand or antisense strand of a target gene of interest, said region having a nucleic acid sequence of at least 50% sequence identity, based on the Clustal V method of alignment, when compared to said all or part of a sense strand or antisense strand from which said region is derived, and wherein said target gene of interest encodes a LPK polypeptide, and wherein said plant exhibits an alteration of at least one agronomic characteristic when compared to a control plant not comprising said recombinant DNA construct.

3. A plant (for example, a maize or soybean plant) comprising in its genome a recombinant DNA construct comprising a polynucleotide operably linked to at least one regulatory sequence, wherein said polynucleotide encodes a LPK protein, and wherein said plant exhibits an altered root architecture when compared to a control plant not comprising said recombinant DNA construct. The plant may further exhibits an alteration of at least one agronomic characteristic.

The LPK protein may be from Arabidopsis thaliana, Zea mays, Glycine max, Glycine tabacina, Glycine soja or Glycine tomentella.

4. A plant (for example, a maize or soybean plant) comprising in its genome a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a LPK protein, and wherein said plant exhibits an alteration of at least one agronomic characteristic when compared to a control plant not comprising said recombinant DNA construct.

5. A plant (for example, a maize or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (b) a full complement of the nucleic acid sequence of (a), and wherein said plant exhibits an alteration of at least one agronomic characteristic when compared to a control plant not comprising said recombinant DNA construct.

6. Any progeny of the above plants in embodiments 1-5, any seeds of the above plants in embodiments 1-5, any seeds of progeny of the above plants in embodiments 1-5, and cells from any of the above plants in embodiments 1-5 and progeny thereof.

In any of the foregoing embodiments 1-6 or any other embodiments of the present invention, the recombinant DNA construct (or suppression DNA construct) may comprise at least a promoter that is functional in a plant as a regulatory sequence.

In any of the foregoing embodiments 1-6 or any other embodiments of the present invention, the alteration of at least one agronomic characteristic is either an increase or decrease.

In any of the foregoing embodiments 1-6 or any other embodiments of the present invention, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, stalk lodging, plant height, ear length, ear height, harvest index, salt tolerance, early seedling vigor and seedling emergence under low temperature stress. For example, the alteration of at least one agronomic characteristic may be an increase in yield, greenness, biomass or root lodging.

In any of the foregoing preferred embodiments 1-6 or any other embodiments of the present invention, the plant may exhibit the alteration of at least one agronomic characteristic irrespective of the environmental conditions, for example, water and nutrient availability, when compared to a control plant.

One of ordinary skill in the art is familiar with protocols for determining alteration in plant root architecture. For example, transgenic maize plants can be assayed for changes in root architecture at seedling stage, flowering time or maturity. Alterations in root architecture can be determined by counting the nodal root numbers of the top 3 or 4 nodes of the greenhouse grown plants or the width of the root band. “Root band” refers to the width of the mat of roots at the bottom of a pot at plant maturity. Other measures of alterations in root architecture include, but are not limited to, the number of lateral roots, average root diameter of nodal roots, average root diameter of lateral roots, number and length of root hairs. The extent of lateral root branching (e.g. lateral root number, lateral root length) can be determined by sub-sampling a complete root system, imaging with a flat-bed scanner or a digital camera and analyzing with WinRHIZO™ software (Regent Instruments Inc.).

Data taken on root phenotype are subjected to statistical analysis, normally a t-test to compare the transgenic roots with that of non-transgenic sibling plants. One-way ANOVA may also be used in cases where multiple events and/or constructs are involved in the analysis.

The Examples below describe some representative protocols and techniques for detecting alterations in root architecture.

One can also evaluate alterations in root architecture by the ability of the plant to increase yield in field testing when compared, under the same conditions, to a control or reference plant.

One can also evaluate alterations in root architecture by the ability of the plant to maintain substantial yield (for example, at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under stress conditions (e.g., nutrient over-abundance or limitation, water over-abundance or limitation, presence of disease), when compared to the yield of a control or reference plant under non-stressed conditions.

Alterations in root architecture can also be measured by determining the resistance to root lodging of the transgenic plants compared to reference or control plants.

One of ordinary skill in the art would readily recognize a suitable control or reference plant to be utilized when assessing or measuring an agronomic characteristic or phenotype of a transgenic plant in any embodiment of the present invention in which a control or reference plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the parent inbred or variety line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

### Methods

Methods include but are not limited to methods for altering root architecture in a plant, methods for evaluating alteration of root architecture in a plant, methods for altering an agronomic characteristic in a plant, methods for determining an alteration of an agronomic characteristic in a plant, and methods for producing seed. The plant may be a monocotyledonous or dicotyledonous plant, such as a maize or soybean plant. The plant may also be sunflower, sorghum, castor bean, canola, wheat, alfalfa, cotton, rice, barley or millet. The seed may be a maize or soybean seed, or a maize hybrid seed or maize inbred seed.

Additional methods include but are not limited to the following:

A method of altering root architecture of a plant, comprising: (a) introducing into a regenerable plant cell a recombinant DNA construct comprising a polynucleotide operably linked to at least one regulatory sequence (such as a promoter functional in a plant), wherein the polynucleotide encodes a polypeptide having an amino acid sequence of at least 50%, 51′)/0, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70; and (b) regenerating a transgenic plant from the regenerable plant cell after step (a), wherein the transgenic plant comprises in its genome the recombinant DNA construct and exhibits altered root architecture when compared to a control plant not comprising the recombinant DNA construct. The method may further comprise (c) obtaining a progeny plant derived from the transgenic plant, wherein said progeny plant comprises in its genome the recombinant DNA construct and exhibits altered root architecture when compared to a control plant not comprising the recombinant DNA construct.

A method of altering root architecture in a plant, comprising: (a) introducing into a regenerable plant cell a suppression DNA construct comprising at least one regulatory sequence (such as a promoter functional in a plant) operably linked to:

(i) all or part of: (A) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (B) a full complement of the nucleic acid sequence of (a)(i)(A); or

(ii) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a LPK polypeptide; and

(b) regenerating a transgenic plant from the regenerable plant cell after step (a), wherein the transgenic plant comprises in its genome the recombinant DNA construct and exhibits an altered root architecture when compared to a control plant not comprising the suppression DNA construct. The method may further comprise (c) obtaining a progeny plant derived from the transgenic plant, wherein said progeny plant comprises in its genome the recombinant DNA construct and exhibits altered root architecture when compared to a control plant not comprising the suppression DNA construct.

A method of evaluating altered root architecture in a plant, comprising (a) introducing into a regenerable plant cell a recombinant DNA construct comprising a polynucleotide operably linked to at least on regulatory sequence (such as a promoter functional in a plant), wherein the polynucleotide encodes a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (b) regenerating a transgenic plant from the regenerable plant cell after step (a), wherein the transgenic plant comprises in its genome the recombinant DNA construct; and (c) evaluating root architecture of the transgenic plant compared to a control plant not comprising the recombinant DNA construct. The method may further comprise (d) obtaining a progeny plant derived from the transgenic plant, wherein the progeny plant comprises in its genome the recombinant DNA construct; and (e) evaluating root architecture of the progeny plant compared to a control plant not comprising the recombinant DNA construct.

A method of evaluating altered root architecture in a plant, comprising (a) introducing into a regenerable plant cell a suppression DNA construct comprising at least one regulatory sequence (such as a promoter functional in a plant) operably linked to:

(i) all or part of: (A) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (B) a full complement of the nucleic acid sequence of (a)(i)(A); or (ii) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a LPK polypeptide; and

(b) regenerating a transgenic plant from the regenerable plant cell after step (a), wherein the transgenic plant comprises in its genome the suppression DNA construct; and (c) evaluating the transgenic plant for altered root architecture compared to a control plant not comprising the suppression DNA construct. The method may further comprise (d) obtaining a progeny plant derived from the transgenic plant, wherein the progeny plant comprises in its genome the suppression DNA construct; and (e) evaluating the progeny plant for altered root architecture compared to a control plant not comprising the suppression DNA construct.

A method of evaluating altered root architecture in a plant, comprising (a) introducing into a regenerable plant cell a recombinant DNA construct comprising a polynucleotide operably linked to at least one regulatory sequence (such as a promoter functional in a plant), wherein said polynucleotide encodes a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO 27, 29, 35, 37, 39, 41, 43, 46, 47, 48, 49, 50, 51, 52, 53, 54, 55, 56, 57, 58, 59, 68 or 70 (b) regenerating a transgenic plant from the regenerable plant cell after step (a), wherein the transgenic plant comprises in its genome the recombinant DNA construct; (c) obtaining a progeny plant derived from said transgenic plant, wherein the progeny plant comprises in its genome the recombinant DNA construct; and (d) evaluating the progeny plant for altered root architecture compared to a control plant not comprising the recombinant DNA construct.

A method of evaluating root architecture in a plant, comprising:

(a) introducing into a regenerable plant cell a suppression DNA construct comprising at least one regulatory element operably linked to: (i) all or part of: (A) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (B) a full complement of the nucleic acid sequence of (a)(i)(A); or (ii) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a LPK or polypeptide; (b) regenerating a transgenic plant from the regenerable plant cell after step (a), wherein the transgenic plant comprises in its genome the suppression DNA construct;
(c) obtaining a progeny plant derived from the transgenic plant, wherein the progeny plant comprises in its genome the suppression DNA construct; and (d) evaluating root architecture of the progeny plant compared to a control plant not comprising the suppression DNA construct.

A method of determining an alteration of an agronomic characteristic in a plant, comprising (a) introducing into a regenerable plant cell a recombinant DNA construct comprising a polynucleotide operably linked to at least on regulatory sequence (such as a promoter functional in a plant), wherein said polynucleotide encodes a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70 (b) regenerating a transgenic plant from the regenerable plant cell after step (a), wherein the transgenic plant comprises in its genome said recombinant DNA construct; and (c) determining whether the transgenic plant exhibits an alteration of at least one agronomic characteristic when compared to a control plant not comprising the recombinant DNA construct. The method may further comprise (d) obtaining a progeny plant derived from the transgenic plant, wherein the progeny plant comprises in its genome the recombinant DNA construct; and (e) determining whether the progeny plant exhibits an alteration of at least one agronomic characteristic when compared to a control plant not comprising the recombinant DNA construct.

A method of determining an alteration of an agronomic characteristic in a plant, comprising (a) introducing into a regenerable plant cell a suppression DNA construct comprising at least one regulatory sequence (such as a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (ii) a full complement of the nucleic acid sequence of (i); (b) regenerating a transgenic plant from the regenerable plant cell after step (a), wherein the transgenic plant comprises in its genome the suppression DNA construct; and (c) determining whether the transgenic plant exhibits an alteration in at least one agronomic characteristic when compared to a control plant not comprising the suppression DNA construct. The method may further comprise (d) obtaining a progeny plant derived from the transgenic plant, wherein the progeny plant comprises in its genome the suppression DNA construct; and (e) determining whether the progeny plant exhibits an alteration in at least one agronomic characteristic when compared to a control plant not comprising the suppression DNA construct.

A method of determining an alteration of an agronomic characteristic in a plant, comprising (a) introducing into a regenerable plant cell a recombinant DNA construct comprising a polynucleotide operably linked to at least one regulatory sequence (such as a promoter functional in a plant), wherein said polynucleotide encodes a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70 (b) regenerating a transgenic plant from the regenerable plant cell after step (a), wherein the transgenic plant comprises in its genome said recombinant DNA construct; (c) obtaining a progeny plant derived from said transgenic plant, wherein the progeny plant comprises in its genome the recombinant DNA construct; and (d) determining whether the progeny plant exhibits an alteration of at least one agronomic characteristic when compared to a control plant not comprising the recombinant DNA construct. The method of determining an alteration of an agronomic characteristic in a plant may further comprise determining whether the transgenic plant exhibits an alteration of at least one agronomic characteristic when compared, under varying environmental conditions, to a control plant not comprising the recombinant DNA construct.

A method of determining an alteration of an agronomic characteristic in a plant, comprising (a) introducing into a regenerable plant cell a suppression DNA construct comprising at least one regulatory sequence (such as a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, or (ii) a full complement of the nucleic acid sequence of (i);

(b) regenerating a transgenic plant from the regenerable plant cell after step (a), wherein the transgenic plant comprises in its genome the suppression DNA construct; (c) obtaining a progeny plant derived from said transgenic plant, wherein the progeny plant comprises in its genome the suppression DNA construct; and (d) determining whether the progeny plant exhibits an alteration in at least one agronomic characteristic when compared to a control plant not comprising the recombinant DNA construct.

A method of determining an alteration of an agronomic characteristic in a plant, comprising: (a) introducing into a regenerable plant cell a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a LPK polypeptide; (b) regenerating a transgenic plant from the regenerable plant cell after step (a), wherein the transgenic plant comprises in its genome the suppression DNA construct; and (c) determining whether the transgenic plant exhibits an alteration of at least one agronomic characteristic when compared to a control plant not comprising the suppression DNA construct. The method may further comprise: (d) obtaining a progeny plant derived from the transgenic plant, wherein the progeny plant comprises in its genome the suppression DNA construct; and (e) determining whether the progeny plant exhibits an alteration of at least one agronomic characteristic when compared to a control plant not comprising the suppression DNA construct.

A method of determining an alteration of an agronomic characteristic in a plant, comprising: (a) introducing into a regenerable plant cell a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a LPK polypeptide; (b) regenerating a transgenic plant from the regenerable plant cell after step (a), wherein the transgenic plant comprises in its genome the suppression DNA construct; (c) obtaining a progeny plant derived from the transgenic plant, wherein the progeny plant comprises in its genome the suppression DNA construct; and (d) determining whether the progeny plant exhibits an alteration of at least one agronomic characteristic when compared to a control plant not comprising the suppression DNA construct.

A method of producing seed (such as seed that can be sold as a product offering with altered root architecture) comprising any of the preceding methods, and further comprising obtaining seeds from said progeny plant, wherein said seeds comprise in their genome said recombinant DNA construct (or suppression DNA construct).

In any of the foregoing methods or any other embodiments of methods of the present invention, the step of determining an alteration of an agronomic characteristic in a transgenic plant, if applicable, may comprise determining whether the transgenic plant exhibits an alteration of at least one agronomic characteristic when compared, under varying environmental conditions, to a control plant not comprising the recombinant DNA construct.

In any of the foregoing methods or any other embodiments of methods of the present invention, the step of determining an alteration of an agronomic characteristic in a progeny plant, if applicable, may comprise determining whether the progeny plant exhibits an alteration of at least one agronomic characteristic when compared, under varying environmental conditions, to a control plant not comprising the recombinant DNA construct.

In any of the preceding methods or any other embodiments of methods of the present invention, in said introducing step said regenerable plant cell may comprise a callus cell (for example an embryogenic callus cell), a gametic cell, a meristematic cell, or a cell of an immature embryo. The regenerable plant cells may be from an inbred maize plant.

In any of the preceding methods or any other embodiments of methods of the present invention, said regenerating step may comprise: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present invention, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, for example as part of a transposable element), and then screen for an event in which the regulatory sequence is operably linked to an endogenous gene encoding a polypeptide of the instant invention.

The introduction of recombinant DNA constructs of the present invention into plants may be carried out by any suitable technique, including but not limited to direct DNA uptake, chemical treatment, electroporation, microinjection, cell fusion, infection, vector mediated DNA transfer, bombardment, or Agrobacterium-mediated transformation. Techniques for plant transformation and regeneration have been described in International Patent Publication WO 2009/006276, the contents of which are herein incorporated by reference.

In any of the preceding methods or any other embodiments of methods of the present invention, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, whole plant free amino acid content, fruit free amino acid content, seed free amino acid content, fruit protein content, seed protein content, protein content in a vegetative tissue, drought tolerance, nitrogen uptake, root lodging, harvest index, stalk lodging, plant height, ear height, ear length, early seedling vigor and seedling emergence under low temperature stress. The increase may be in yield, greenness, biomass or root lodging.

In any of the preceding methods or any other embodiments of methods of the present invention, the plant may exhibit the alteration of at least one agronomic characteristic irrespective of the environmental conditions when compared to a control.

The introduction of recombinant DNA constructs of the present invention into plants may be carried out by any suitable technique, including but not limited to direct DNA uptake, chemical treatment, electroporation, microinjection, cell fusion, infection, vector mediated DNA transfer, bombardment, or Agrobacterium mediated transformation.

Techniques for transformation of maize plant cells and soybean plant cells are set forth in the Examples below.

Other include methods for transforming dicots, primarily by use of Agrobacterium tumefaciens, and obtaining transgenic plants include those published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011, McCabe et. al., Bio/Technology 6:923 (1988), Christou et al., Plant Physiol. 87:671 674 (1988)); Brassica (U.S. Pat. No. 5,463,174); peanut (Cheng et al., Plant Cell Rep. 15:653 657 (1996), McKently et al., Plant Cell Rep. 14:699 703 (1995)); papaya; and pea (Grant et al., Plant Cell Rep. 15:254 258, (1995)).

Transformation of monocotyledons using electroporation, particle bombardment, and Agrobacterium have also been reported and are included as methods for use in the invention, for example, transformation and plant regeneration as achieved in asparagus (Bytebier et al., Proc. Natl. Acad. Sci. U.S.A. 84:5354, (1987)); barley (Wan and Lemaux, Plant Physiol. 104:37 (1994)); Zea mays (Rhodes et al., Science 240:204 (1988), Gordon-Kamm et al., Plant Cell 2:603 618 (1990), Fromm et al., Bio/Technology 8:833 (1990), Koziel et al., Bio/Technology 11:194, (1993), Armstrong et al., Crop Science 35:550-557 (1995)); oat (Somers et al., Bio/Technology 10:1589 (1992)); orchard grass (Horn et al., Plant Cell Rep. 7:469 (1988)); rice (Toriyama et al., Theor. Appl. Genet. 205:34, (1986); Part et al., Plant Mol. Biol. 32:1135 1148, (1996); Abedinia et al., Aust. J. Plant Physiol. 24:133 141 (1997); Zhang and Wu, Theor. Appl. Genet. 76:835 (1988); Zhang et al., Plant Cell Rep. 7:379, (1988); Battraw and Hall, Plant Sci. 86:191 202 (1992); Christou et al., Bio/Technology 9:957 (1991)); rye (De la Pena et al., Nature 325:274 (1987)); sugarcane (Bower and Birch, Plant J. 2:409 (1992)); tall fescue (Wang et al., Bio/Technology 10:691 (1992)), and wheat (Vasil et al., Bio/Technology 10:667 (1992); U.S. Pat. No. 5,631,152).

There are a variety of methods for the regeneration of plants from plant tissue. The particular method of regeneration will depend on the starting plant tissue and the particular plant species to be regenerated.

The regeneration, development, and cultivation of plants from single plant protoplast transformants or from various transformed explants is well known in the art (Weissbach and Weissbach, In: Methods for Plant Molecular Biology, (Eds.), Academic Press, Inc. San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

In another aspect, this invention also concerns a method of mapping genetic variations related to altering root architecture and/or altering at least one agronomic characteristic in plants comprising:

(a) crossing two plant varieties; and

(b) evaluating genetic variations with respect to:
(i) a nucleic acid sequence selected from the group consisting of SEQ ID NO: 24, 26, 28, 30, 32, 34, 36, 38, 67, or 69; or
(ii) a nucleic acid sequence encoding a polypeptide selected from the group consisting of SEQ ID NO: 25, 27, 29, 31, 33, 40, 41, 68, or 70 in progeny plants resulting from the cross of step (a) wherein the evaluation is made using a method selected from the group consisting of: RFLP analysis, SNP analysis, and PCR-based analysis.

In another embodiment, this invention concerns a method of molecular breeding to obtain an altered root architecture and/or at least one altered agronomic characteristic in plants comprising:

(a) crossing two plant varieties; and

(b) evaluating genetic variations with respect to:
(i) a nucleic acid sequence selected from the group consisting of SEQ ID NO: 24, 26, 28, 30, 32, 34, 36, 38, 67, or 69; or
(ii) a nucleic acid sequence encoding a polypeptide selected from the group consisting of SEQ ID NO: 25, 27, 29, 31, 33, 40, 41, 68, or 70;
   in progeny plants resulting from the cross of step (a) wherein the evaluation is made using a method selected from the group consisting of: RFLP analysis, SNP analysis, and PCR-based analysis.

The terms “mapping genetic variation” or “mapping genetic variability” are used interchangeably and define the process of identifying changes in DNA sequence, whether from natural or induced causes, within a genetic region that differentiates between different plant lines, cultivars, varieties, families, or species. The genetic variability at a particular locus (gene) due to even minor base changes can alter the pattern of restriction enzyme digestion fragments that can be generated. Pathogenic alterations to the genotype can be due to deletions or insertions within the gene being analyzed or even single nucleotide substitutions that can create or delete a restriction enzyme recognition site. RFLP analysis takes advantage of this and utilizes Southern blotting with a probe corresponding to the isolated nucleic acid fragment of interest.

Thus, if a polymorphism (i.e., a commonly occurring variation in a gene or segment of DNA; also, the existence of several forms of a gene (alleles) in the same species) creates or destroys a restriction endonuclease cleavage site, or if it results in the loss or insertion of DNA (e.g., a variable nucleotide tandem repeat (VNTR) polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms: (“RFLPs”). RFLPs have been widely used in human and plant genetic analyses (Glassberg, UK Patent Application 2135774; Skolnick et al, Cytogen. Cell Genet. 32:58-67 (1982); Botstein et al, Ann. J. Hum. Genet. 32:314-331 (1980); Fischer et al (PCT Application WO 90/13668; Uhlen, PCT Application WO 90/11369).

A central attribute of “single nucleotide polymorphisms” or “SNPs” is that the site of the polymorphism is at a single nucleotide. SNPs have certain reported advantages over RFLPs or VNTRs. First, SNPs are more stable than other classes of polymorphisms. Their spontaneous mutation rate is approximately 10⁻⁹ (Kornberg, DNA Replication, W.H. Freeman & Co., San Francisco, 1980), approximately, 1,000 times less frequent than VNTRs (U.S. Pat. No. 5,679,524). Second, SNPs occur at greater frequency, and with greater uniformity than RFLPs and VNTRs. As SNPs result from sequence variation, new polymorphisms can be identified by sequencing random genomic or cDNA molecules. SNPs can also result from deletions, point mutations and insertions. Any single base alteration, whatever the cause, can be a SNP. The greater frequency of SNPs means that they can be more readily identified than the other classes of polymorphisms.

SNPs can be characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism or by other biochemical interpretation. SNPs can be sequenced by a number of methods. Two basic methods may be used for DNA sequencing, the chain termination method of Sanger et al, Proc. Natl. Acad. Sci. (U.S.A.) 74:5463-5467 (1977), and the chemical degradation method of Maxam and Gilbert, Proc. Natl. Acad. Sci. (U.S.A.) 74: 560-564 (1977).

Furthermore, single point mutations can be detected by modified PCR techniques such as the ligase chain reaction (“LCR”) and PCR-single strand conformational polymorphisms (“PCR-SSCP”) analysis. The PCR technique can also be used to identify the level of expression of genes in extremely small samples of material, e.g., tissues or cells from a body. The technique is termed reverse transcription-PCR (“RT-PCR”).

The term “molecular breeding” defines the process of tracking molecular markers during the breeding process. It is common for the molecular markers to be linked to phenotypic traits that are desirable. By following the segregation of the molecular marker or genetic trait, instead of scoring for a phenotype, the breeding process can be accelerated by growing fewer plants and eliminating assaying or visual inspection for phenotypic variation. The molecular markers useful in this process include, but are not limited to, any marker useful in identifying mapable genetic variations previously mentioned, as well as any closely linked genes that display synteny across plant species. The term “synteny” refers to the conservation of gene placement/order on chromosomes between different organisms. This means that two or more genetic loci, that may or may not be closely linked, are found on the same chromosome among different species. Another term for synteny is “genome colinearity”.

### Examples

The present invention is further illustrated in the following Examples, in which parts and percentages are by weight and degrees are Celsius, unless otherwise stated. It should be understood that these Examples are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Creation of an Arabidopsis Population with Activation-Tagged Genes

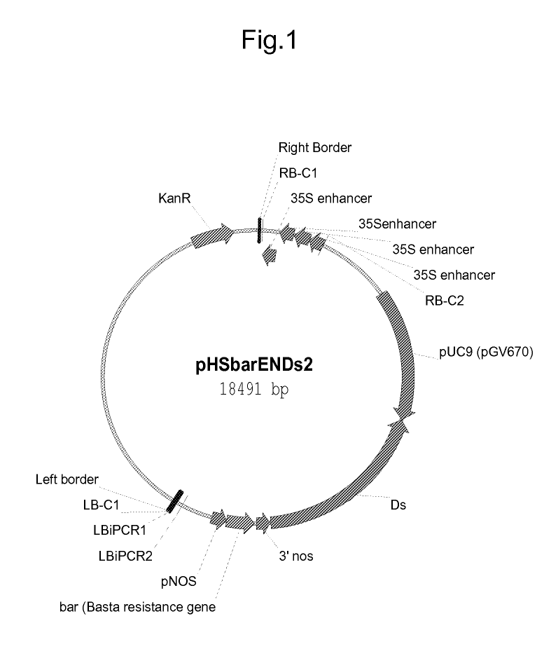

A 18.4 kb T-DNA based binary construct was created, pHSbarENDs2 (FIG. 1; SEQ ID NO:1;) containing four multimerized enhancer elements derived from the Cauliflower Mosaic Virus 35S promoter, corresponding to sequences −341 to −64, as defined by Odell et al. (1985) Nature 313:810-812. The construct also contains vector sequences (pUC9) to allow plasmid rescue, transposon sequences (Ds) to remobilize the T-DNA, and the bar gene to allow for glufosinate selection of transgenic plants. Only the 10.8 kb segment from the right border (RB) to left border (LB) inclusive will be transferred into the host plant genome. Since the enhancer elements are located near the RB, they can induce cis-activation of genomic loci following T-DNA integration.

The pHSbarENDs2 construct was transformed into Agrobacterium tumefaciens strain C58, grown in LB at 25° C. to OD600 ˜1.0. Cells were then pelleted by centrifugation and resuspended in an equal volume of 5% sucrose/0.05% Silwet L-77 (OSI Specialties, Inc). At early bolting, soil grown Arabidopsis thaliana ecotype Col-0 were top watered with the Agrobacterium suspension. A week later, the same plants were top watered again with the same Agrobacterium strain in sucrose/Silwet. The plants were then allowed to set seed as normal. The resulting T₁ seed were sown on soil, and transgenic seedlings were selected by spraying with glufosinate (Finale®; AgrEvo; Bayer Environmental Science). T₂ seed was collected from approximately 35,000 individual glufosinate resistant T₁ plants. T₂ plants were grown and equal volumes of T₃ seed from 96 separate T₂ lines were pooled. This constituted 360 sub-populations.

The Agrobacterium strain and whole plant transformation was performed as described above.

A total of 100,000 glufosinate resistant T₁ seedlings were selected. T₂ seed from each line was kept separate.

### Example 2A

### Screens to Identify Lines with Altered Root Architecture Non-Limiting Nitrogen Conditions

Activation-tagged Arabidopsis seedlings, grown under non-limiting nitrogen conditions, can be analyzed for altered root system architecture when compared to control seedlings during early development from the population described in Example 1.

From each of 96,000 separate T1 activation-tagged lines, ten T2 seeds can be sterilized with chlorine gas and planted on petri plates containing the following medium: 0.5×N-Free Hoagland's, 60 mM KNO₃, 0.1% sucrose, 1 mM MES and 1% Phytagel™. Typically 10 plates are placed in a rack. Plates are kept for three days at 4° C. to stratify seeds and then held vertically for 11 days at 22° C. light and 20° C. dark. Photoperiod is 16 h; 8 h dark, average light intensity was ˜180 μmol/m²/s. Racks (typically holding 10 plates each) are rotated daily within each shelf. At day 14, plates are evaluated for seedling status, whole plate digital images were taken, and analyzed for root area. Plates are arbitrarily divided in 10 horizontal areas. The root area in each of 10 horizontal zones on the plate is expressed as a percentage of the total area. Only areas in zones 3 to 9 are used to calculate the total root area of the line. Rootbot image analysis tool (proprietary) developed by ICORIA can be used to assess root area. Total root area is expressed in mm².

Lines with enhanced root growth characteristics are expected to lie at the upper extreme of the root area distributions. A sliding window approach can be used to estimate the variance in root area for a given rack with the assumption that there could be up to two outliers in the rack. Environmental variations in various factors including growth media, temperature, and humidity can cause significant variation in root growth, especially between sow dates. Therefore the lines are grouped by sow date and shelf for the data analysis. The racks in a particular sow date/shelf group are then sorted by mean root area. Root area distributions for sliding windows is performed by combining data for a rack, rᵢ, with data from the rack with the next lowest, (rₒ, and the next highest mean root area, rᵢ₊₁. The variance of the combined distribution is then analyzed to identify outliers in rᵢ using a Grubbs-type approach (Barnett et al., Outliers in Statistical Data, John Wiley & Sons, 3^{rd} edition (1994).

Lines with significant enhanced root growth as determined by the method outlined above, are designated as Phase 1 hits. Phase 1 hits are re-screened in duplicate under the same assay conditions. When either or both of the Phase 2 replicates shows a significant difference from the mean, the line is then considered a validated root architecture line.

Those lines that were again found to be outliers in at least one plate in Phase 2 were subjected to a Phase 3 screening performed in house, to validate the results obtained in Phase 1 and Phase 2. The results were validated in Phase 3 using both the Rootboot image analysis (as described above) and WinRHIZO®, as described below. The confirmation is performed in the same fashion as in the first round of screening. T2 seeds are sterilized using 75% EtOH 0.01% triton X-100 solution and plated onto the same plate medium as described in the first round of screening at a density of 4 seeds/plate, 8 plates per line. Plates are kept for three days at 4° C. to stratify seeds, and grown in the same temperature and photoperiod as the first experiment with the light intensity ˜160 μmol/m²/s. Plates are placed vertically into the eight center positions of a 10 plate rack with the first and last position holding blank plates. The racks and the plates within the rack are rotated every other day. Plants are imaged at 14 days of growth and these images are used for image analysis. These seedlings grown on vertical plates are analyzed for root growth with the software WinRHIZO® (regent Instruments Inc.), an image analysis system specifically designed for root measurement. WinRHIZO® uses the contrast pixels to distinguish the light root from the darker background. To identify the maximum amount of roots without picking up background, the pixel classification is 150-170 and the filter feature is used to remove objects that have a length/width ration less than 10.0. The area on the plates analyzed from is from the edge of the plant's leaves to about 1 cm from the bottom of the plate. The exact same WinRHIZO® settings and area of analysis are used to analyze all plates within a batch. The total root length score given by WinRHIZO® for a plate is divided by the number of plants that have germinated and have grown halfway down the plate. Eight plates for every line are grown and their scores are averaged. This average is then compared to the average of eight plates containing pooled T2 seeds taken from random lines that are grown at the same time.

### Example 2B

### Identification of Mutant Lines with an Altered Root Phenotype in a Mutant Population Limiting Nitrogen Conditions

A two-step screening procedure can be used, comprising:

(1) Identification of an altered root growth phenotype in a vertical plate assay;

(2) Confirm herbicide resistance and root phenotype in rescued mutant lines; The primary screen is based on vertical plates containing Nitrogen-free Hoagland salts, 0.3% sucrose and 1 mM KNO₃. The media also contains 0.8%-1.0% PhytaGel as a gelling agent. Media with Phytagel at 1.0% is sometimes difficult to pour as it solidifies quickly, however, at below 0.8% the media will slide off plates when placed vertically. Mutants from an activation-tagged population where pools of 100 lines each are available for a total of 36000 lines are being screened. On each plate, 12 mutant and 2 wild type Columbia seeds are seeded. Plates are placed in a growth room with a constant temperature of 26° C., 16 hr-day cycle with an average of 110 pE/m²s light intensity at the top of the plates. These plates are photographed 3-4 times in a 2.5 week time frame. Individual seedlings are rescued when a clear root phenotype is observed. Rescued seedlings are grown to maturity in a growth chamber (24° C., 16 hr day, 250-300 pE/m²s) for seed collection.

For the secondary screening, seeds from putative hits identified in the primary screen are sowed on plates containing the same media as above plus 6 mg/L bialaphos. Wild type Columbia seeds are sown at the same time on the same media but without bialaphos. Each plate has 10 seeds. There are 3 plates for each mutant line, and 2 plates for wild type Columbia, as replication. These plates are placed under the same growth conditions as described above in a growth room. Those lines that do not have herbicide resistance or no obvious root phenotype are discarded as false positives. Lines validated by the second screen are saved for further study.

### Example 3

### Identification of Activation-Tagged Genes

Genes flanking the T-DNA insert in lines with altered root architecture are identified using one, or both, of the following two standard procedures: (1) thermal asymmetric interlaced (TAIL) PCR (Liu et al., (1995), Plant J. 8:457-63); and (2) SAIFF PCR (Siebert et al., (1995) Nucleic Acids Res. 23:1087-1088). In lines with complex multimerized T-DNA inserts, TAIL PCR and SAIFF PCR may both prove insufficient to identify candidate genes. In these cases, other procedures, including inverse PCR, plasmid rescue and/or genomic library construction, can be employed.

A successful result is one where a single TAIL or SAIFF PCR fragment contains a T-DNA border sequence and Arabidopsis genomic sequence.

Once a tag of genomic sequence flanking a T-DNA insert is obtained, candidate genes are identified by alignment to publicly available Arabidopsis genome sequence.

Specifically, the annotated gene nearest the 35S enhancer elements/T-DNA RB are candidates for genes that are activated.

To verify that an identified gene is truly near a T-DNA and to rule out the possibility that the TAIL/SAIFF fragment is a chimeric cloning artifact, a diagnostic PCR on genomic DNA is done with one oligo in the T-DNA and one oligo specific for the candidate gene. Genomic DNA samples that give a PCR product are interpreted as representing a T-DNA insertion. This analysis also verifies a situation in which more than one insertion event occurs in the same line, e.g., if multiple differing genomic fragments are identified in TAIL and/or SAIFF PCR analyses.

### Example 4

### Identification of Activation-Tagged lpk Gene

The lpk gene was obtained by the screening procedure as described in Example 2A and subsequently subjected to a phase 3 (in house) screening as described in Example 2A. Identification of the activation-tagged gene was performed as described in Example 3.

One line displaying altered root architecture was further analyzed. DNA from the line was extracted and the T-DNA insertion was found by ligation mediated PCR (Siebert et al., (1995) Nucleic Acids Res. 23:1087-1088) using primers within the LeftBorder of the T-DNA. Once a tag of genomic sequence flanking a T-DNA insert was obtained, the candidate gene was identified by sequence alignment to the completed Arabidopsis genome. One of the insertion sites identified was identified as a chimeric insertion; Left Border T-DNA sequence was determined to be at both ends of the T-DNA insertion. It is still possible that the enhancer elements located near the Right Border of the T-DNA are close enough to have an effect on the nearby candidate gene. In this case the location of the Right Border was assumed to be present at the insertion site, and the two genes that flank the insertion site were chosen as candidates. One of the genes nearest the 35S enhancers of the chimeric insertion was AT5G60270 (SEQ ID NO:44); Arabidopsis thaliana lpk domain-containing protein, encoding the LPK protein (SEQ ID NO:46), referred herein as LPK domain-containing protein (LPK).

### Example 5A

### Validation of a Candidate Arabidopsis Gene (AT5G60270) for its Ability to Enhance Root Architecture in Plants Via Transformation into Arabidopsis

Candidate genes can be transformed into Arabidopsis and overexpressed under the 35S promoter. If the same or similar phenotype is observed in the transgenic line as in the parent activation-tagged line, then the candidate gene is considered to be a validated “lead gene” in Arabidopsis.

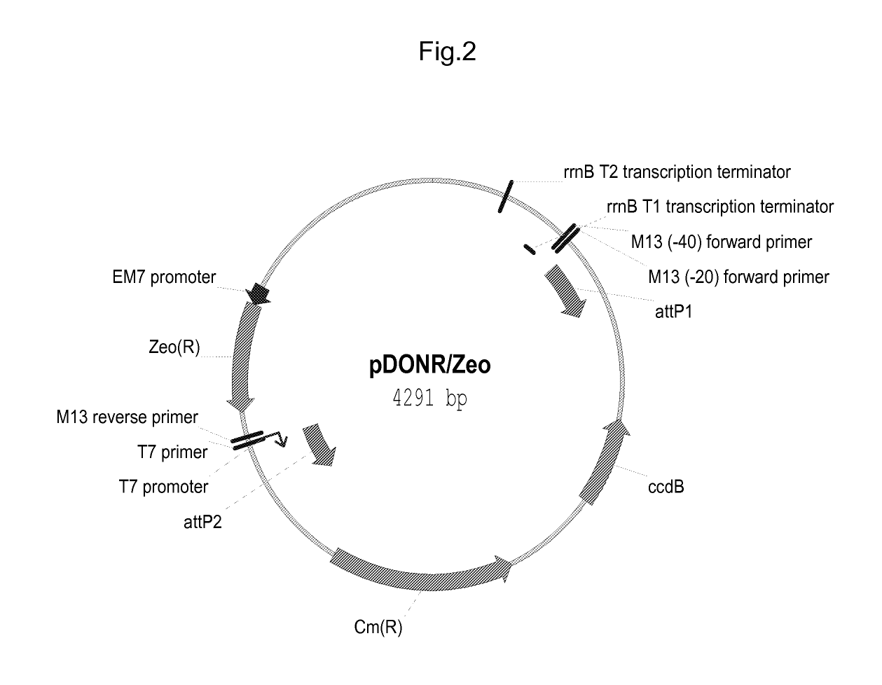

The Arabidopsis AT5G60270 gene can be directly tested for its ability to enhance root architecture in Arabidopsis.
The Arabidopsis AT5G60270 cDNA was PCR amplified with oligos that introduce the attB1 (SEQ ID NO:14) sequence, a consensus start sequence (CAACA) upstream of the ATG start codon and the first 21 nucleotides of the protein coding-region of the AT5G60270 cDNA (SEQ ID NO:45) and the attB2 (SEQ ID NO:15) sequence and the last 21 nucleotides of the protein-coding region including the stop codon of said cDNA. Using Invitrogen™ Gateway® technology a MultiSite Gateway® BP Recombination Reaction was performed with pDONR™/Zeo (Invitrogen™, FIG. 2; SEQ ID NO:2). This process removes the bacteria lethal ccdB gene, as well as the chloramphenicol resistance gene (CAM) from pDONR™/Zeo and directionally clones the PCR product with flanking attB1 (SEQ ID NO:14) and attB2 (SEQ ID NO:15) sites creating entry clone PHP30211.

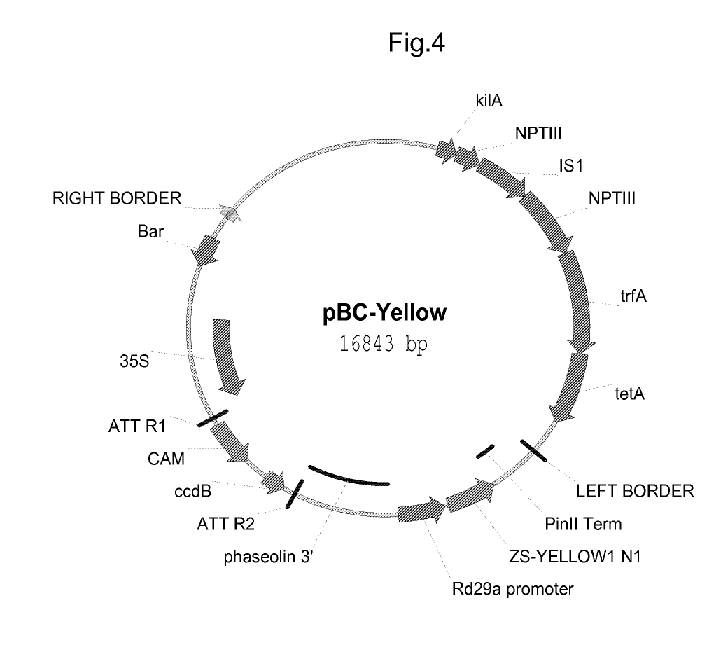

A 16.8-kb T-DNA based binary vector, called pBC-yellow (FIG. 4, SEQ ID NO:4), was constructed with the 1.3-kb 35S promoter immediately upstream of the Invitrogen™ Gateway® C1 conversion insert containing the ccdB gene and the chloramphenicol resistance gene (CAM) flanked by attR1 and attR2 sequences. The vector also contains a YFP marker under the control of the Rd29a promoter for the selection of transformed seeds.

Using Invitrogen™ Gateway® technology a MultiSite Gateway® LR Recombination Reaction was performed on the entry clone containing the directionally cloned PCR product and pBC-yellow. This allowed rapid and directional cloning of the AT5G60270 gene behind the 35S promoter in pBC-yellow.

The 35S— AT5G60270 gene construct was introduced into wild-type Arabidopsis ecotype Col-0, using the same Agrobacterium-mediated transformation procedure described in Example 1.

Transgenic T1 plants were selected by the presence of the fluorescent YFP marker in the seed coat or herbicide selection. Fluorescent seeds were subjected to the Root Architecture Assay following the procedure described in Example 2A. Transgenic T1 seeds were re-screened using 6 plates per construct. Two plates per rack containing non-transformed Columbia seed discarded from fluorescent seed sorting served as a control.

Six plates per construct were analyzed statistically and a trend was detected between the number of plant growing on a plate and their average WinRHIZO®score. WinRHIZO®scores were normalized for this trend and the root score corresponding to the construct was divided by the wild-type root score.

### Example 5B

### Screen of Candidate Genes Under Nitrogen Limiting Conditions

Transgenic T1 seed selected by the presence of the fluorescent marker YFP as described above in Example 5A can also be screened for their tolerance to grow under nitrogen limiting conditions. For this purpose 32 transgenic individuals can be grown next to 32 wild-type individuals on one plate with either 0.4 mM KNO₃ or 60 mM KNO₃. If a line shows a statistically significant difference from the controls, the line is considered a validated nitrogen-deficiency tolerant line. After masking the plate image to remove background color, two different measurements are collected for each individual: total rosetta area, and the percentage of color that falls into a green color bin. Using hue, saturation and intensity data (HIS), the green color bin consists of hues 50-66. Total rosetta area is used as a measure of plant biomass, whereas the green color bin has been shown by dose-response studies to be an indicator of nitrogen assimilation.

### Example 5C

### Screen to Identify Lines with Improved Nitrate Uptake

For each overexpressor line, twelve T2 plants are sown on 96 well micro titer plates containing 2 mM MgSO₄, 0.5 mM KH₂PO₄, 1 mM CaCl₂, 2.5 mM KCl, 0.15 mM Sprint 330, 0.06 mM FeSO₄, 1 μM MnCl₂ 4H₂O, 1 μM ZnSO₄ 7H₂O, 3 μM H₃BO₃, 0.1 μM NaMoO₄, 0.1 μM CuSO₄ 5H₂O,

0.8 mM potassium nitrate, 0.1% sucrose, 1 mM MES, 200 μM bromophenol red and 0.40% Phytagel™ (pH assay medium). The pH of the medium is so that the color of bromophenol is red, the pH indicator dye, is yellow.

Four lines are plated per plate, and the inclusion of 12 wild-type individuals and 12 individuals from a line that has shown an improvement in nitrate uptake (positive control) on each plate makes for a total of 72 individuals on each 96 well micro titer plate A web-based random sequence generator can be used to determine the order of the lines on each plate. Seeds are not plated in Row A or Row H on the 96 well micro titer plate. Four plates are plated for each experiment, resulting in a maximum of 48 plants per line analyzed. Plates are kept for three days in the dark at 4° C. to stratify seeds, and then placed horizontally for six days at 22° C. light and dark. Photoperiod is sixteen hours light; eight hours dark, with an average light intensity of ˜200 mmol/m²/s. Plates are rotated and shuffled within each shelf. At day eight or nine (five or six days of growth), seedling status is evaluated by recording the color of the medium as pink, peach, yellow or no germination. Then the plants and/or seeds are removed from each well. Each medium plug is transferred to 1.2 ml micro titer tubes and placed in the corresponding well in a 96 well deep micro titer plate. An equal volume of water containing 2 μM flourescein is added to each 1.2 ml micro titer tube. The plate is covered with foil and autoclaved on liquid cycle. Each tube is mixed well, and an aliquot is removed from each tube and analyzed for amount of nitrate remaining in the medium. If t-test shows that a line is significantly different (p<0.05) from wild-type control, the line is then considered a validated improved nitrate uptake line.

### Example 6

### Composition of cDNA Libraries; Isolation and Sequencing of cDNA Clones

cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred and Phrap (Ewing et al. (1998) Genome Res. 8:175-185; Ewing and Green (1998) Genome Res. 8:186-194). Phred is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (Gordon et al. (1998) Genome Res. 8:195-202).

In some of the clones the cDNA fragment corresponds to a portion of the 3′-terminus of the gene and does not cover the entire open reading frame. In order to obtain the upstream information one of two different protocols are used. The first of these methods results in the production of a fragment of DNA containing a portion of the desired gene sequence while the second method results in the production of a fragment containing the entire open reading frame. Both of these methods use two rounds of PCR amplification to obtain fragments from one or more libraries. The libraries some times are chosen based on previous knowledge that the specific gene should be found in a certain tissue and some times are randomly-chosen. Reactions to obtain the same gene may be performed on several libraries in parallel or on a pool of libraries. Library pools are normally prepared using from 3 to 5 different libraries and normalized to a uniform dilution. In the first round of amplification both methods use a vector-specific (forward) primer corresponding to a portion of the vector located at the 5′-terminus of the clone coupled with a gene-specific (reverse) primer. The first method uses a sequence that is complementary to a portion of the already known gene sequence while the second method uses a gene-specific primer complementary to a portion of the 3′-untranslated region (also referred to as UTR). In the second round of amplification a nested set of primers is used for both methods. The resulting DNA fragment is ligated into a pBluescript vector using a commercial kit and following the manufacturer's protocol. This kit is selected from many available from several vendors including Invitrogen™ (Carlsbad, Calif.), Promega Biotech (Madison, Wis.), and Gibco-BRL (Gaithersburg, Md.). The plasmid DNA is isolated by alkaline lysis method and submitted for sequencing and assembly using Phred/Phrap, as above.

### Example 7

### Identification of cDNA Clones

cDNA clones encoding LPK polypeptides were identified by conducting BLAST (Basic Local Alignment Search Tool; Altschul et al. (1993) J. Mol. Biol. 215:403-410; see also the explanation of the BLAST algorithm on the world wide web site for the National Center for Biotechnology Information at the National Library of Medicine of the National Institutes of Health) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained as described in Example 6 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis are compared to the Genbank database as described above. ESTs that contain sequences more 5- or 3-prime can be found by using the BLASTn algorithm (Altschul et al (1997) Nucleic Acids Res. 25:3389-3402.) against the Du Pont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described in Example 6. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy. In cases where the sequence assemblies are in fragments, the percent identity to other homologous genes can be used to infer which fragments represent a single gene. The fragments that appear to belong together can be computationally assembled such that a translation of the resulting nucleotide sequence will return the amino acid sequence of the homologous protein in a single open-reading frame. These computer-generated assemblies can then be aligned with other polypeptides of the invention. Alternatively the Velvet assembled fragments can be run against the dataset of the original trimmed reads with SSAKE (René L Warren, Granger G Sutton, Steven J M Jones, Robert A Holt. 2007 (epub 2006 Dec. 8). Assembling millions of short DNA sequences using SSAKE. Bioinformatics. 23:500-501.) SSAKE can be run with modified overlap parameters in order to extend the assembly with the trimmed reads.

### Example 8

### Characterization of cDNA Clones Encoding LPK Polypeptides

cDNA libraries representing mRNAs from various tissues of Arabidopsis, Maize, and soybean were prepared as described in Example 6. The characteristics of the libraries are described in Table 2.

TABLE 2
cDNA Libraries from, Maize

| Library | Tissue | Clone |
|---|---|---|
| adf2c | Arabidopsis Landsberg erecta flowers at | adf2c.pk002.h2 |
| | anthesis (DAF 0-1) | |
| cfp6n | Maize Leaf and Seed pooled, Full-length | cfp6n.pk065.d16:fis |
| | enriched normalized | |
| p0050 | Maize. | p0050.cjlae47r |
| p0110 | Maize. | p0110.cgsmp02r |
| sbacm | Soybean. | sbacm.pk094.p8.f |

The BLASTX search using the EST sequences from clones listed in Table 1 and SEQ ID NO:46 revealed similarity of the polypeptides encoded by the cDNAs to LPK polypeptides from Maize (GI NOs: 226528693, and 226502714 corresponding to SEQ ID NOs:47, and 48, respectively, Sorghum (Gi NOs: 242032451 and 242095594 corresponding to SEQ ID NOs:49 and 52, respectively), Medicago trunculata)Gi NO.:38112427) and from Arabidopsis (GI NOs: 15239261 and 15239260, respectively). Shown in Table 3 are the percent identity results for the sequences of the entire cDNA inserts (“Full-Insert Sequence” or “FIS”) of the clones listed in Table 2. Each cDNA insert encodes an entire functional protein (“Complete Gene Sequence” or “CGS”). Also shown in Tables 3 and 4 are the percent sequence identity values per each pair of amino acid sequences using the Clustal V method of alignment with default parameters.

TABLE 1
BLASTP Results and Percent Identity for Sequences Encoding
Polypeptides Homologous to LPK Polypeptides

| | | NCBI GI No./ | | |
|---|---|---|---|---|
| | | gene | | |
| Sequence | Status | accession no. | E-value | % identity |
| p0110.cgsmp02r | CGS | 226528693 | 0.00e+0 | 100 |
| SEQ ID NO: 27 | | (Zea mays) | | |
| | | SEQ ID NO: 47 | | |
| cfp6n.pk065.d16:fis | CGS | 226502714 | 0.00e+0 | 100 |
| SEQ ID NO: 29 | | (Zea mays) | | |
| | | SEQ ID NO: 48 | | |
| Pred1 | CGS | 242032451 | 0.00e+0 | 83.6 |
| SEQ ID NO: 35 | | (Sorghum | | |
| | | bicolor) | | |
| | | SEQ ID NO: 49 | | |
| Pred2 | CGS | 38112427 | 8.70e−297 | 99.3 |
| SEQ ID NO: 37 | | (Medicago | | |
| | | trunculata) | | |
| | | SEQ ID NO: 50 | | |
| Pred3 | CGS | 15239261 | 0.00e+0 | 100 |
| SEQ ID NO: 39 | | (Arabidopsis | | |
| | | thaliana) | | |
| | | SEQ ID NO: 51 | | |
| Custom6.pk880.d9 | CGS | 242095594 | 0.00e+0 | 85.5 |
| (SEQ ID NO: 41) | | (Sorghum | | |
| | | bicolor) | | |
| | | SEQ ID NO: 52 | | |
| Custom5.pk735.d4 | CGS | 15239260 | 0.00e+0 | 100 |
| (SEQ ID NO: 43) | | (Arabidopsis | | |
| | | thaliana) | | |
| | | SEQ ID NO: 53 | | |
| Resurrection grass | CGS | XP_002461846 | 0.00e+0 | 78.2 |
| 38031 | | (Sorghum | | |
| (SEQ ID NO: 68) | | bicolor) | | |
| | | SEQ ID NO: 71 | | |
| Resurrection grass | CGS | XP_002441536 | 0.00e+0 | 92.4 |
| 175180 | | (Sorghum | | |
| (SEQ ID NO: 70) | | bicolor) | | |
| | | SEQ ID NO: 72 | | |

Pred 1, 2, and 3 were obtained using long range genomic PCR capture and analyzed using the FGENESH program. Additionally, the sequence was aligned with sequences from other species, and manually edited to remove putative introns. Primers designed based on the genomic locus of Pred1, 2, and 3 were used for long range genomic PCR capture. The resulting PCR product was sequenced and the FGENESH program and manually editing was used to predict the coding sequence of pred1, 2, and 3 (SEQ ID NO: 34, 36 and 38, respectively).

FIGS. 15A through 15K show the multiple alignment of the full length amino acid sequences of SEQ ID NOs: 27, 29, 35, 37, 39, 41, 43, 46, 47, 48, 49, 50, 51, 52, 53, 54, 55, 56, 57, 58, and 59. FIG. 16 presents the percent sequence identities and divergence values for each sequence pair presented in FIGS. 15A through 15K.

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Sequence alignments and percent identity indicate that the nucleic acid fragments comprising the instant cDNA clones encode LPK polypeptides.

TABLE 4
BLASTP Results and Percent identity for Sequences Encoding
Polypeptides Homologous to LPK polypeptides

| Sequence | Status | Reference | E-value | % identity |
|---|---|---|---|---|
| p0110.cgsmp02r | CGS | SEQ ID NO: | 1.30e−271 | 68.1 |
| SEQ ID NO: 27 | | 138302 | | |
| | | (Oryza sativa) in | | |
| | | EP2090662 | | |
| | | (SEQ ID NO: 54) | | |
| cfp6n.pk065.d16:fis | CGS | SEQ ID NO: | 0.00e+0 | 100 |
| SEQ ID NO: 29 | | 69317 | | |
| | | (Zea mays) | | |
| | | EP2090662 | | |
| | | (SEQ ID NO: 55) | | |
| Pred1 | CGS | SEQ ID NO: | 2.00e−259 | 65 |
| SEQ ID NO: 35 | | 138302 | | |
| | | (Oryza sativa) in | | |
| | | EP2090662 | | |
| | | (SEQ ID NO: 54) | | |
| Pred2 | CGS | SEQ ID NO: | 0.00e+0 | 99.3 |
| SEQ ID NO: 37 | | 193726 | | |
| | | (Glycine max) in | | |
| | | EP2090662 | | |
| | | (SEQ ID NO: 56) | | |
| Pred3 | CGS | SEQ ID NO: | 0.00e+0 | 100 |
| SEQ ID NO: 39 | | 167274 | | |
| | | (Arabidopsis | | |
| | | thaliana) | | |
| | | in EP2090662 | | |
| | | (SEQ ID NO: 57) | | |
| Custom6.pk0880.d9 | CGS | SEQ ID NO: | 0.00e+0 | 97.6 |
| SEQ ID NO: 41 | | 167322 | | |
| | | (Zea mays) in | | |
| | | EP2090662 | | |
| | | (SEQ ID NO: 58) | | |
| Custom5.pk735.d4 | CGS | SEQ ID NO: | 0.00e+0 | 100 |
| SEQ ID NO: 43 | | 167272 | | |
| | | (Arabidopsis | | |
| | | thaliana) | | |
| | | in EP2090662 | | |
| | | (SEQ ID NO: 59) | | |

### Example 9

### Preparation of a Plant Expression Vector Containing a Homolog of the Arabidopsis Lead Gene (AT5G60270)

Sequences homologous to the lead lpk gene can be identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Homologous lpk sequences, such as the ones described in Example 8, can be PCR-amplified by either of the following methods.

Method 1 (RNA-based): If the 5′ and 3′ sequence information for the protein-coding region of a LPK polypeptide homolog is available, gene-specific primers can be designed as outlined in Example 5A. RT-PCR can be used with plant RNA to obtain a nucleic acid fragment containing the lpk protein-coding region flanked by attB1 (SEQ ID NO:14) and attB2 (SEQ ID NO:15) sequences. The primer may contain a consensus Kozak sequence (CAACA) upstream of the start codon.

Method 2 (DNA-based): Alternatively, if a cDNA clone is available for a gene encoding a LPK polypeptide homolog, the entire cDNA insert (containing 5′ and 3′ non-coding regions) can be PCR amplified. Forward and reverse primers can be designed that contain either the attB1 sequence and vector-specific sequence that precedes the cDNA insert or the attB2 sequence and vector-specific sequence that follows the cDNA insert, respectively. For a cDNA insert cloned into the vector pBluescript SK-F, the forward primer VC062 (SEQ ID NO:16) and the reverse primer VC063 (SEQ ID NO:17) can be used.

Methods 1 and 2 can be modified according to procedures known by one skilled in the art. For example, the primers of method 1 may contain restriction sites instead of attB1 and attB2 sites, for subsequent cloning of the PCR product into a vector containing attB1 and attB2 sites. Additionally, method 2 can involve amplification from a cDNA clone, a lambda clone, a BAC clone or genomic DNA.

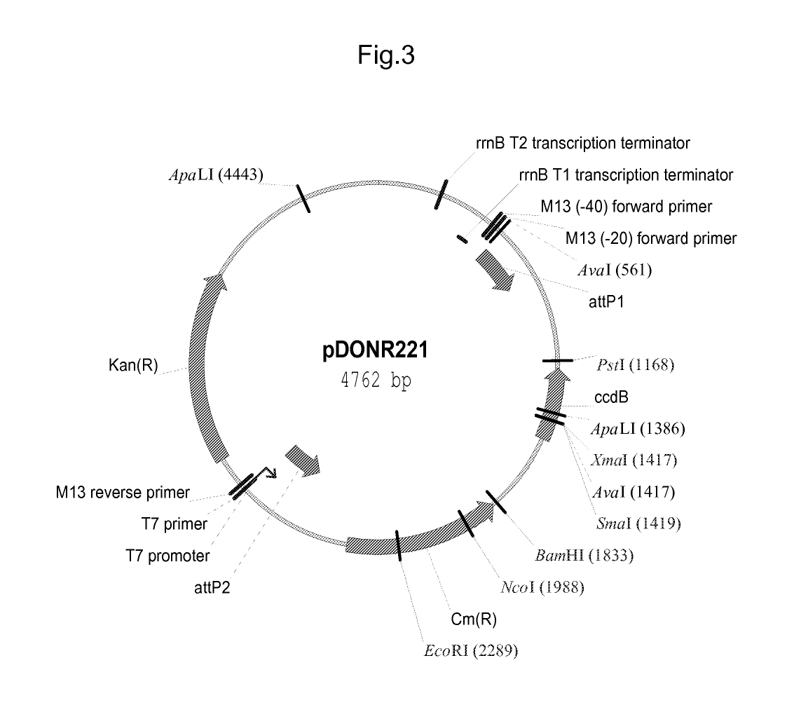

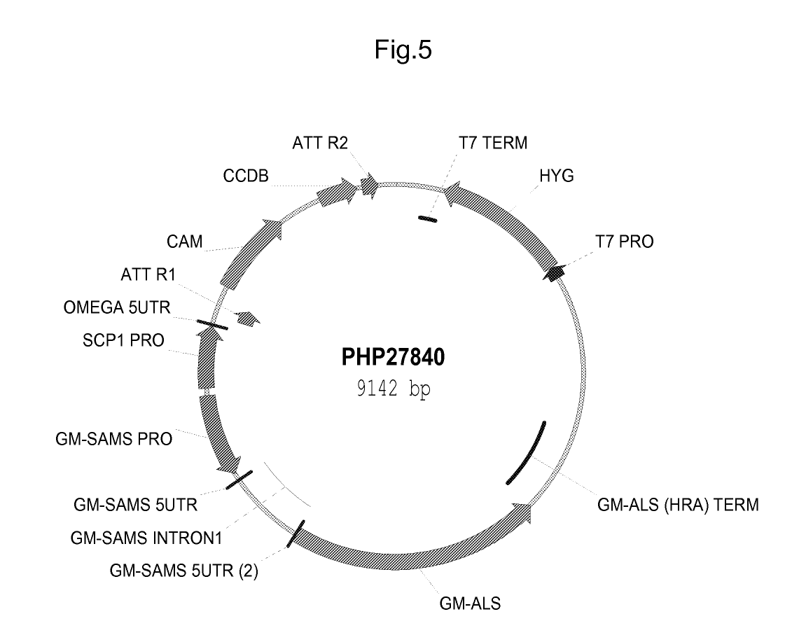

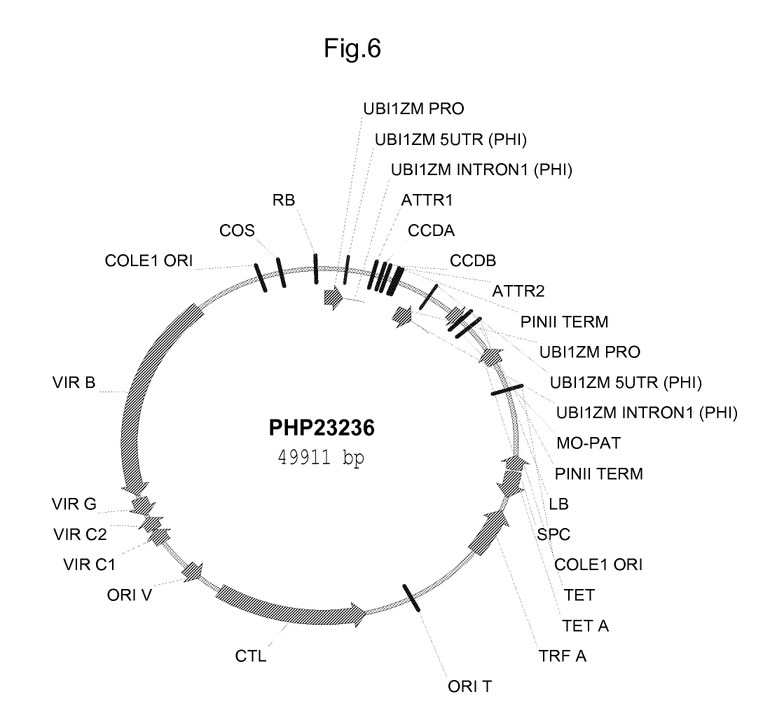

A PCR product obtained by either method above can be combined with the Gateway® donor vector, such as pDONR™/Zeo (Invitrogen™, FIG. 2; SEQ ID NO:2) or pDONR™221 (Invitrogen™, FIG. 3; SEQ ID NO:3) using a BP Recombination Reaction. This process removes the bacteria lethal ccdB gene, as well as the chloramphenicol resistance gene (CAM) from pDONR™221 and directionally clones the PCR product with flanking attB1 and attB2 sites to create an entry clone. Using the Invitrogen™ Gateway® Clonase™ technology, the homologous lpk gene from the entry clone can then be transferred to a suitable destination vector to obtain a plant expression vector for use with Arabidopsis, corn and soy, such as pBC-Yellow (FIG. 4; SEQ ID NO:4), PHP27840 (FIG. 5; SEQ ID NO:5) or PHP23236 (FIG. 6; SEQ ID NO:6), to obtain a plant expression vector for use with Arabidopsis, soybean and corn, respectively.

Alternatively a MultiSite Gateway® LR recombination reaction between multiple entry clones and a suitable destination vector can be performed to create an expression vector. An Example of this procedure is outlined in Example 14A, describing the construction of maize expression vectors for transformation of maize lines.

### Example 10

### Preparation of Soybean Expression Vectors and Transformation of Soybean with Validated Arabidopsis Lead Genes and Homologs Thereof

Soybean plants can be transformed to overexpress the validated Arabidopsis gene (AT5G60270) and the corresponding homologs from various species in order to examine the resulting phenotype.

The entry clones described in Example 5A and 9 can be used to directionally clone each gene into PHP27840 vector (FIG. 5, SEQ ID NO:5) such that expression of the gene is under control of the SCP1 promoter.

Soybean embryos may then be transformed with the expression vector comprising sequences encoding the instant polypeptides.

To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos, which produce secondary embryos, are then excised and placed into a suitable liquid medium. After repeated selection for clusters of somatic embryos which multiply as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium. Soybean embryogenic suspension cultures may then be transformed by the method of particle gun bombardment (Klein et al. (1987) Nature (London) 327:70-73, U.S. Pat. No. 4,945,050). A DuPont Biolistic™ PDS1000/HE instrument (helium retrofit) can be used for these transformations.

A selectable marker gene which can be used to facilitate soybean transformation is a chimeric gene composed of the 35S promoter from cauliflower mosaic virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. Another selectable marker gene which can be used to facilitate soybean transformation is an herbicide-resistant acetolactate synthase (ALS) gene from soybean or Arabidopsis. ALS is the first common enzyme in the biosynthesis of the branched-chain amino acids valine, leucine and isoleucine. Mutations in ALS have been identified that convey resistance to some or all of three classes of inhibitors of ALS (U.S. Pat. No. 5,013,659; the entire contents of which are herein incorporated by reference). Expression of the herbicide-resistant ALS gene can be under the control of a SAM synthetase promoter (U.S. Patent Application No. US-2003-0226166-A1; the entire contents of which are herein incorporated by reference).

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

Enhanced root architecture can be measured in soybean by growing the plants in soil and wash the roots before analysis of the total root mass with WinRHIZO®.

Soybean plants transformed with validated genes can then be assayed to study agronomic characteristics relative to control or reference plants. For example, nitrogen utilization efficacy, yield enhancement and/or stability under various environmental conditions (e.g. nitrogen limiting conditions, drought etc.)

### Example 11

### Transformation of Maize with Validated Arabidopsis Lead Genes Using Particle Bombardment

Maize plants can be transformed to overexpress a validated Arabidopsis lead gene or the corresponding homologs from various species in order to examine the resulting phenotype.

The Gateway® entry clones described in Example 5A can be used to directionally clone each gene into a maize transformation vector. Expression of the gene in maize can be under control of a constitutive promoter such as the maize ubiquitin promoter (Christensen et al., Plant Mol. Biol. 12:619-632 (1989) and Christensen et al., Plant Mol. Biol. 18:675-689 (1992))

The recombinant DNA construct described above can then be introduced into maize cells by the following procedure. Immature maize embryos can be dissected from developing caryopses derived from crosses of the inbred maize lines H99 and LH132. The embryos are isolated ten to eleven days after pollination when they are 1.0 to 1.5 mm long. The embryos are then placed with the axis-side facing down and in contact with agarose-solidified N6 medium (Chu et al., Sci. Sin. Peking 18:659-668 (1975)). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every two to three weeks.

The plasmid, p35S/Ac (obtained from Dr. Peter Eckes, Hoechst Ag, Frankfurt, Germany) may be used in transformation experiments in order to provide for a selectable marker. This plasmid contains the pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from cauliflower mosaic virus (Odell et al., Nature 313:810-812 (1985)) and the 3′ region of the nopaline synthase gene from the T-DNA of the Ti plasmid of Agrobacterium tumefaciens.

The particle bombardment method (Klein et al., Nature 327:70-73 (1987)) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After ten minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the maize tissue with a Biolistic® PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains bialaphos (5 mg per liter) and lacks casein or proline. The tissue continues to grow slowly on this medium. After an additional two weeks the tissue can be transferred to fresh N6 medium containing bialaphos. After six weeks, areas of about 1 cm in diameter of actively growing callus can be identified on some of the plates containing the bialaphos-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined following HTP procedures. T1 seed can be collected.

T1 plants can be grown and analyzed for phenotypic changes. The following parameters can be quantified using image analysis: plant area, volume, growth rate and color analysis can be collected and quantified. Expression constructs that result in an alteration of root architecture or any one of the agronomic characteristics listed above compared to suitable control plants, can be considered evidence that the Arabidopsis lead gene functions in maize to alter root architecture or plant architecture.

Furthermore, a recombinant DNA construct containing a validated Arabidopsis gene can be introduced into an maize line either by direct transformation or introgression from a separately transformed line.

Transgenic plants, either inbred or hybrid, can undergo more vigorous field-based experiments to study root or plant architecture, yield enhancement and/or resistance to root lodging under various environmental conditions (e.g. variations in nutrient and water availability).

Subsequent yield analysis can also be done to determine whether plants that contain the validated Arabidopsis lead gene have an improvement in yield performance, when compared to the control (or reference) plants that do not contain the validated Arabidopsis lead gene. Plants containing the validated Arabidopsis lead gene would improve yield relative to the control plants, for example by 50% less yield loss under adverse environmental conditions or would have increased yield relative to the control plants under varying environmental conditions.

### Example 12

### Electroporation of Aqrobacterium tumefaciens LBA4404

Electroporation competent cells (40 μl), such as Agrobacterium tumefaciens LBA4404 (containing PHP10523), are thawn on ice (20-30 min). PHP10523 contains VIR genes for T-DNA transfer, an Agrobacterium low copy number plasmid origin of replication, a tetracycline resistance gene, and a cos site for in vivo DNA biomolecular recombination. Meanwhile the electroporation cuvette is chilled on ice. The electroporator settings are adjusted to 2.1 kV.

A DNA aliquot (0.5 μL JT (U.S. Pat. No. 7,087,812) parental DNA at a concentration of 0.2 μg-1.0 μg in low salt buffer or twice distilled H₂O) is mixed with the thawn Agrobacterium cells while still on ice. The mix is transferred to the bottom of electroporation cuvette and kept at rest on ice for 1-2 min. The cells are electroporated (Eppendorf electroporator 2510) by pushing “Pulse” button twice (ideally achieving a 4.0 msec pulse). Subsequently 0.5 ml 2xYT medium (or SOCmedium) are added to cuvette and transferred to a 15 ml Falcon tube. The cells are incubated at 28-30° C., 200-250 rpm for 3 h.

Aliquots of 250 μl are spread onto #30B (YM+50 μg/mL Spectinomycin) plates and incubated 3 days at 28-30° C. To increase the number of transformants one of two optional steps can be performed:

Option 1: overlay plates with 30 μl of 15 mg/ml Rifampicin. LBA4404 has a chromosomal resistance gene for Rifampicin. This additional selection eliminates some contaminating colonies observed when using poorer preparations of LBA4404 competent cells.
Option 2: Perform two replicates of the electroporation to compensate for poorer electrocompetent cells.

### Identification of Transformants:

Four independent colonies are picked and streaked on AB minimal medium plus 50 mg/mL Spectinomycin plates (#12S medium) for isolation of single colonies. The plated are incubate at 28° C. for 2-3 days.

A single colony for each putative co-integrate is picked and inoculated with 4 ml #60A with 50 mg/l Spectinomycin. The mix is incubated for 24 h at 28° C. with shaking. Plasmid DNA from 4 ml of culture is isolated using Qiagen Miniprep+optional PB wash. The DNA is eluted in 30 μl. Aliquots of 2 μl are used to electroporate 20 μl of DH10b+20 μl of ddH₂O as per above.

Optionally a 15 μl aliquot can be used to transform 75-100 μl of Invitrogen™-Library Efficiency DH5α. The cells are spread on LB medium plus 50 mg/mL Spectinomycin plates (#34T medium) and incubated at 37° C. overnight.

Three to four independent colonies are picked for each putative co-integrate and inoculated 4 ml of 2xYT (#60A) with 50 μg/ml Spectinomycin. The cells are incubated at 37° C. overnight with shaking.

The plasmid DNA is isolated from 4 ml of culture using QiAprep® Miniprep with optional PB wash (elute in 50 μl) and 8 μl are used for digestion with SalI (using JT parent and PHP10523 as controls).

Three more digestions using restriction enzymes BamHI, EcoRI, and HindIII are performed for 4 plasmids that represent 2 putative co-integrates with correct SalI digestion pattern (using parental DNA and PHP10523 as controls). Electronic gels are recommended for comparison.

Alternatively, for high throughput applications, such as described for Gaspe Flint Derived Maize Lines (Examples 15-17), instead of evaluating the resulting co-integrate vectors by restriction analysis, three colonies can be simultaneously used for the infection step as described in Example 13.

### Example 13

### Agrobacterium Mediated Transformation into Maize

Maize plants can be transformed to overexpress a validated Arabidopsis lead gene or the corresponding homologs from various species in order to examine the resulting phenotype.

Agrobacterium-mediated transformation of maize is performed essentially as described by Zhao et al., in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium innoculation, co-cultivation, resting, selection and plant regeneration.

### 1. Immature Embryo Preparation

Immature embryos are dissected from caryopses and placed in a 2 mL microtube containing 2 mL PHI-A medium.

### 2. Agrobacterium Infection and Co-Cultivation of Embryos

### 2.1 Infection Step

PHI-A medium is removed with 1 mL micropipettor and 1 mL Agrobacterium suspension is added. Tube is gently inverted to mix. The mixture is incubated for 5 min at room temperature.

### 2.2 Co-Culture Step

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for 3 days. L-Cysteine can be used in the co-cultivation phase. With the standard binary vector, the co-cultivation medium supplied with 100-400 mg/L L-cysteine is critical for recovering stable transgenic events.

### 3. Selection of Putative Transgenic Events

To each plate of PHI-D medium in a 100×15 mm Petri dish, 10 embryos are transferred, maintaining orientation and the dishes are sealed with Parafilm. The plates are incubated in darkness at 28° C. Actively growing putative events, as pale yellow embryonic tissue are expected to be visible in 6-8 weeks. Embryos that produce no events may be brown and necrotic, and little friable tissue growth is evident. Putative transgenic embryonic tissue is subcultured to fresh PHI-D plates at 2-3 week intervals, depending on growth rate. The events are recorded.

4. Regeneration of T0 plants

Embryonic tissue propagated on PHI-D medium is subcultured to PHI-E medium (somatic embryo maturation medium); in 100×25 mm Petri dishes and incubated at 28° C., in darkness, until somatic embryos mature, for about 10-18 days. Individual, matured somatic embryos with well-defined scutellum and coleoptile are transferred to PHI-F embryo germination medium and incubated at 28° C. in the light (about 80 μE from cool white or equivalent fluorescent lamps). In 7-10 days, regenerated plants, about 10 cm tall, are potted in horticultural mix and hardened-off using standard horticultural methods.

Media for Plant Transformation
1. PHI-A: 4 g/L CHU basal salts, 1.0 mL/L 1000× Eriksson's vitamin mix, 0.5 mg/L thiamin HCL, 1.5 mg/L 2,4-D, 0.69 g/L L-proline, 68.5 g/L sucrose, 36 g/L glucose, pH 5.2. Add 100 μM acetosyringone, filter-sterilized before using.
2. PHI-B: PHI-A without glucose, increased 2,4-D to 2 mg/L, reduced sucrose to 30 g/L and supplemented with 0.85 mg/L silver nitrate (filter-sterilized), 3.0 g/L gelrite, 100 μM acetosyringone (filter-sterilized), 5.8.
3. PHI-C: PHI-B without gelrite and acetosyringonee, reduced 2,4-D to 1.5 mg/L and supplemented with 8.0 g/L agar, 0.5 g/L Ms-morpholino ethane sulfonic acid (MES) buffer, 100 mg/L carbenicillin (filter-sterilized).
4. PHI-D: PHI—C supplemented with 3 mg/L bialaphos (filter-sterilized).

5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, cat. no. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; sucrose reduced to 40 g/L; replacing agar with 1.5 g/L gelrite; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

Phenotypic analysis of transgenic T0 plants and T1 plants can be performed.

T1 plants can be analyzed for phenotypic changes. Using image analysis T1 plants can be analyzed for phenotypical changes in plant area, volume, growth rate and color analysis can be taken at multiple times during growth of the plants. Alteration in root architecture can be assayed as described in Example 20.

Subsequent analysis of alterations in agronomic characteristics can be done to determine whether plants containing the validated Arabidopsis lead gene have an improvement of at least one agronomic characteristic, when compared to the control (or reference) plants that do not contain the validated Arabidopsis lead gene. The alterations may also be studied under various environmental conditions.

Expression constructs that result in a significant alteration in root architecture will be considered evidence that the Arabidopsis gene functions in maize to alter root architecture.

### Example 14A

### Construction of Maize Expression Vectors with the Arabidopsis Lead Gene (AT5G60270) Using Agrobacterium Mediated Transformation

Maize expression vectors can be prepared with the Arabidopsis lpk gene (AT5G60270) under the control of the NAS2 (SEQ ID NO:19) and GOS2 (SEQ ID NO:20) promoter. PINII may be the terminator (SEQ ID NO:23) Using Invitrogen™ Gateway® technology the entry clone, created as described in Example 5A, containing the Arabidopsis lpk gene (AT5G60270) can be used in separate Gateway® LR reactions with:

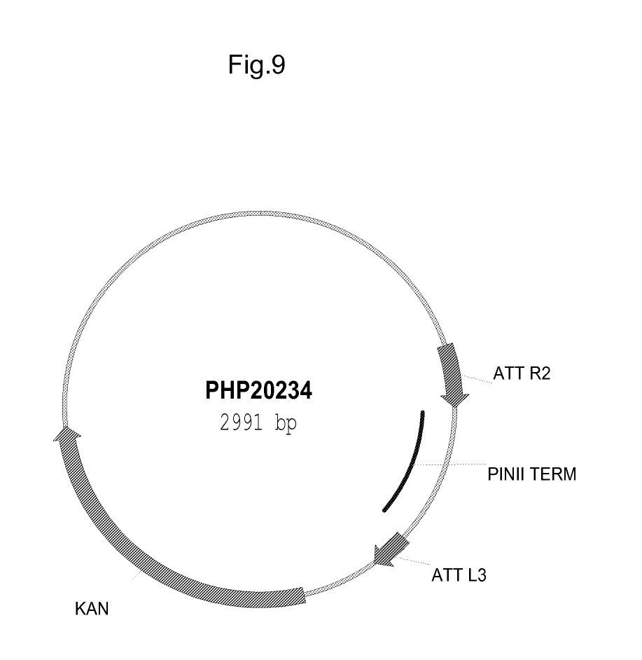

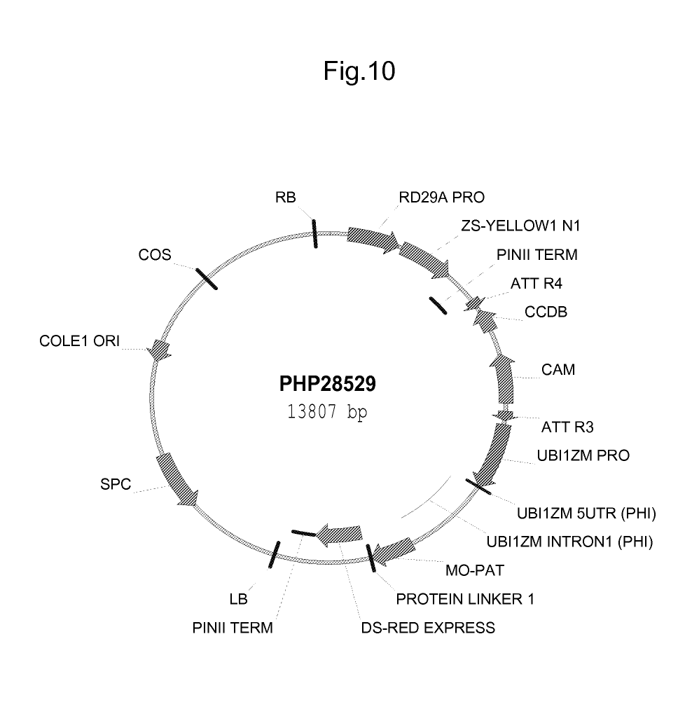

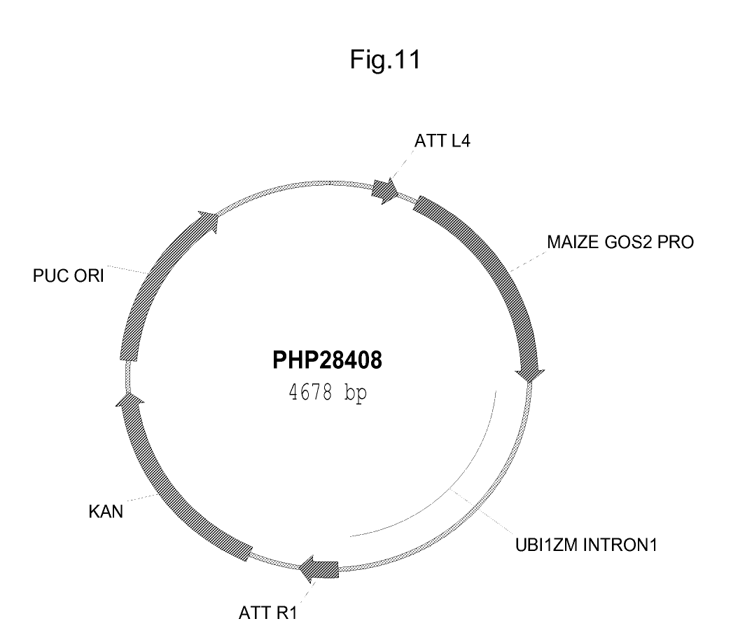

1) the constitutive maize GOS2 promoter entry clone (PHP28408, FIG. 11, SEQ ID NO:11) and the PinII Terminator entry clone (PHP20234, FIG. 9, SEQ ID NO:9) into the destination vector PHP28529 (FIG. 10, SEQ ID NO:10).

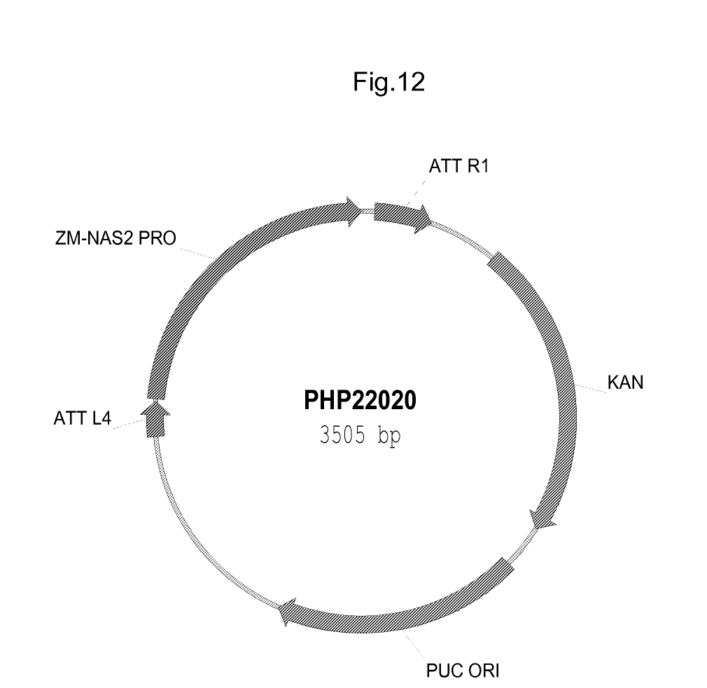

2) the root maize NAS2 promoter entry clone (PHP22020, FIG. 12, SEQ ID NO:12) and the PinII Terminator entry clone (PHP20234, FIG. 9, SEQ ID NO:9) into the destination vector PHP28529 (FIG. 10, SEQ ID NO:10).

The destination vector PHP28529 adds to each of the final vectors (created in steps 1 and 2) also an:
1) RD29A promoter::yellow fluorescent protein::PinII terminator cassette for Arabidospis seed sorting
2) a Ubiquitin promoter::moPAT/red fluorescent protein fusion::PinII terminator cassette for transformation selection and Z. mays seed sorting.

### Example 14B

### Preparation of Maize Expression Constructs Containing the Arabidopsis lpk Gene and Homologs Thereof

The Arabidopsis lpk gene and the corresponding homologs from maize and other species (Table 1 and SEQ ID NO:27) can be transformed into maize lines using the procedures outlined in Examples 5A and 14A. In addition to the GOS2 or NAS2 promoter, other promoters such as, but not limited to the ubiquitin promoter, the S2A and S2B promoter, the maize ROOTMET2 promoter, the maize Cyclo, the CR1BIO, the CRWAQ81 and the maize ZRP2.4447 are useful for directing expression of lpk genes in maize. Furthermore, a variety of terminators, such as, but not limited to the PINII terminator, could be used to achieve expression of the gene of interest in maize.

### Example 14C

### Transformation of Maize Lines with the Arabidopsis Lead Gene (AT5G60270) and Corresponding Homologs from Other Species Using Agrobacterium Mediated Transformation

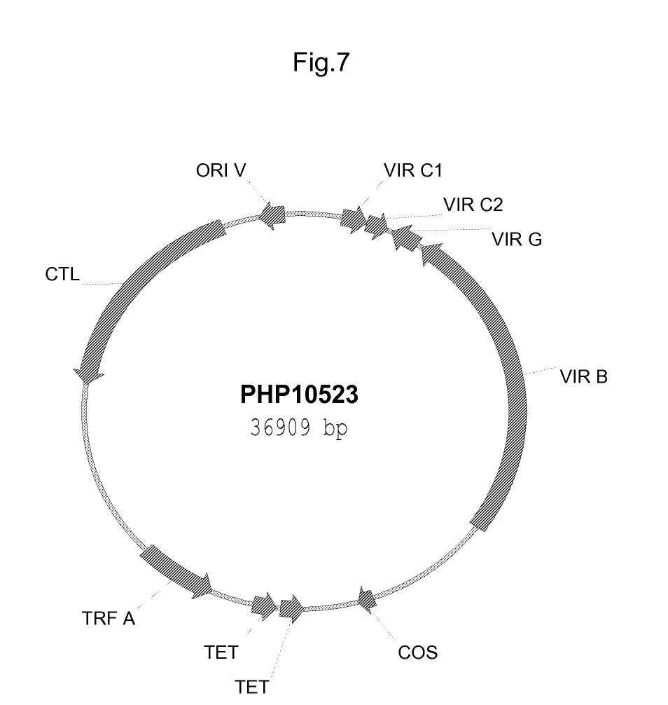

The final vectors (vectors for expression in Maize, Example 14A, and B) can be then electroporated separately into LBA4404 Agrobacterium containing PHP10523 (FIG. 7; SEQ ID NO:7, Komari et al. Plant J 10:165-174 (1996), NCBI GI: 59797027) to create the co-integrate vectors for maize transformation. The co-integrate vectors are formed by recombination of the final vectors (maize expression vectors) with PHP10523, through the COS recombination sites contained on each vector. The co-integrate vectors contain in addition to the expression cassettes described in Examples 14A-B, also genes needed for the Agrobacterium strain and the Agrobacterium mediated transformation, (TET, TET, TRFA, ORI terminator, CTL, ORI V, VIR C1, VIR C2, VIR G, VIR B). Transformation into a maize line can be performed as described in Example 13.

### Example 14D

### Construction of Maize Expression Vectors with the Arabidopsis Lead Gene (AT5G60270) Using Agrobacterium Mediated Transformation

Maize expression vectors can be prepared with the Arabidopsis lpk gene (AT5G60270) under the control of the NAS2 (SEQ ID NO:19) and UBI (SEQ ID NO: 21) promoter. PINII may be the terminator (SEQ ID NO:23).
Using Invitrogen™ Gateway® technology the entry clone, created as described in Example 5A, containing the Arabidopsis lpk gene (AT5G60270) can be used in separate Gateway® LR reactions with:

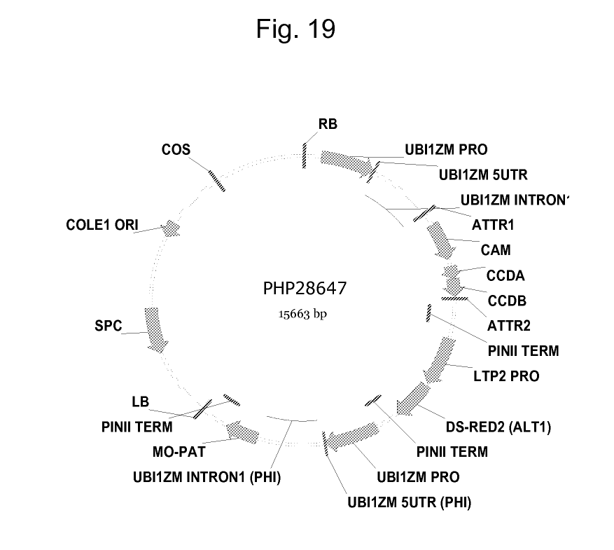

I) the destination vector PHP28647 (FIG. 19, SEQ ID NO:62), which adds the constitutive maize UBI promoter and the PinII Terminator. The destination vector PHP28647 adds to the final vector created above also an:
1) LTP2 promoter::red fluorescent protein::PinII terminator cassette for Z. mays seed sorting
2) a Ubiquitin promoter::moPAT::PinII terminator cassette for transformation selection.
   The resulting vector was named PHP31191.

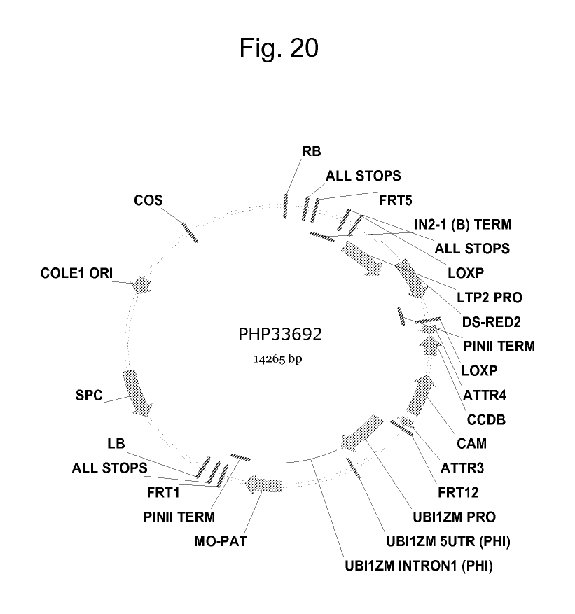

II) the root maize NAS2 promoter entry clone (PHP22020, FIG. 12, SEQ ID NO:12) and the PinII Terminator entry clone (PHP20234, FIG. 9, SEQ ID NO:9) into the destination vector PHP33692 (FIG. 20, SEQ ID NO:63). The destination vector PHP33692 adds to the final vectors created above also an:

1) LTP2 promoter::red fluorescent protein::PinII terminator cassette for Z. mays seed sorting

2) a Ubiquitin promoter::moPAT::PinII terminator cassette for transformation selection.

The resulting vector was named PHP36957.

### Example 15A

### Preparation of the Destination Vectors PHP23236 and PHP29635 and PHP29634 for Transformation of Gaspe Flint Derived Maize Lines

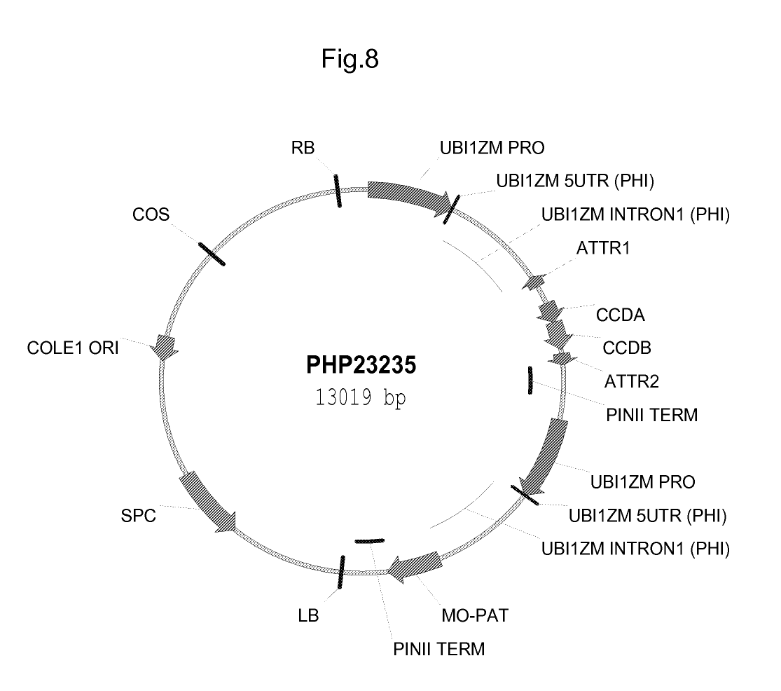

Destination vector PHP23236 (FIG. 6, SEQ ID NO:6) was obtained by transformation of Agrobacterium strain LBA4404 containing plasmid PHP10523 (FIG. 7, SEQ ID NO:7) with plasmid PHP23235 (FIG. 8, SEQ ID NO:8) and isolation of the resulting co-integration product.

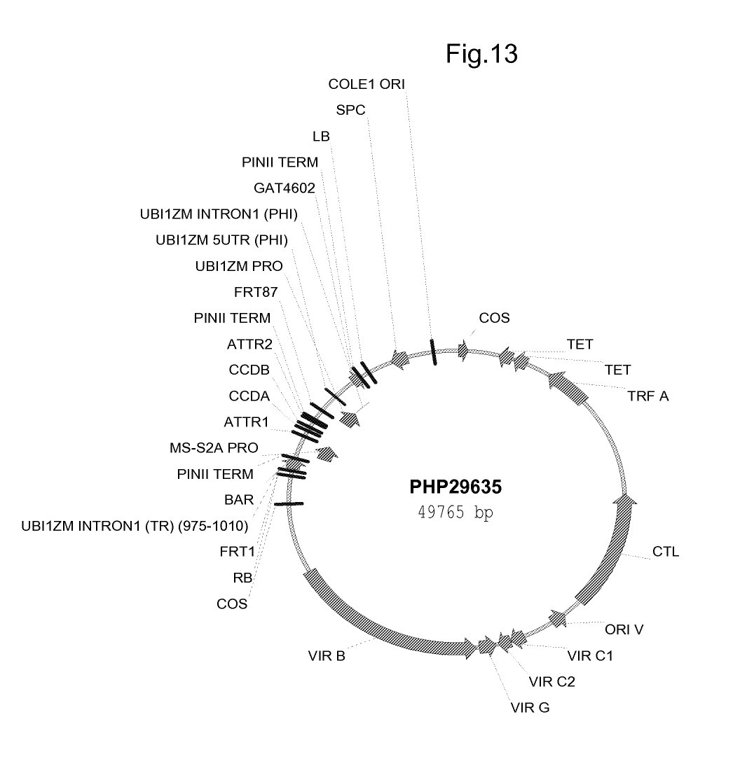

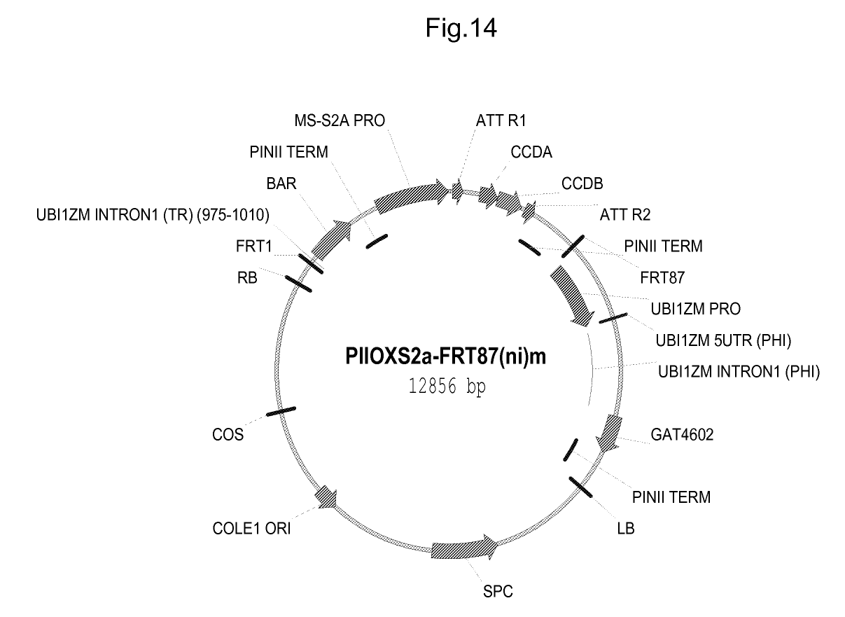

Destination vector PHP23236, can be used in a recombination reaction with an entry clone as described in Example 16 to create a maize expression vector for transformation of Gaspe Flint derived maize lines. Expression of the gene of interest is under control of the ubiquitin promoter (SEQ ID NO:21). PHP29635 (FIG. 13, SEQ ID NO:13) was obtained by transformation of Agrobacterium strain LBA4404 containing plasmid PHP10523 with plasmid PIIOXS2a-FRT87(ni)m (FIG. 14, SEQ ID NO:18) and isolation of the resulting co-integration product. Destination vector PHP29635 can be used in a recombination reaction with an entry clone as described in Example 16 to create a maize expression vector for transformation of Gaspe Flint derived maize lines. Expression of the gene of interest is under control of the S2A promoter (SEQ ID NO:22).

Destination vector PHP29634 is similar to destination vector PHP23236, however, destination vector PHP29634 has site-specific recombination sites FRT1 and FRT87 and also encodes the GAT4602 selectable marker protein for selection of transformants using glyphosate. This expression vector contains the cDNA of interest, encoding AtFeC-II, under control of the UBI promoter and is a T-DNA binary vector for Agrobacterium-mediated transformation into corn as described, but not limited to, the examples described herein.

### Example 15B

### Preparation of the Destination Vectors PHP28647 and PHP33692 for Transformation Maize Lines

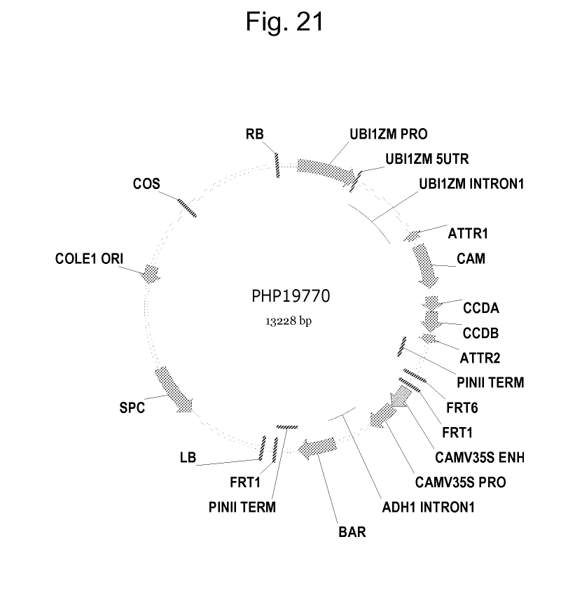

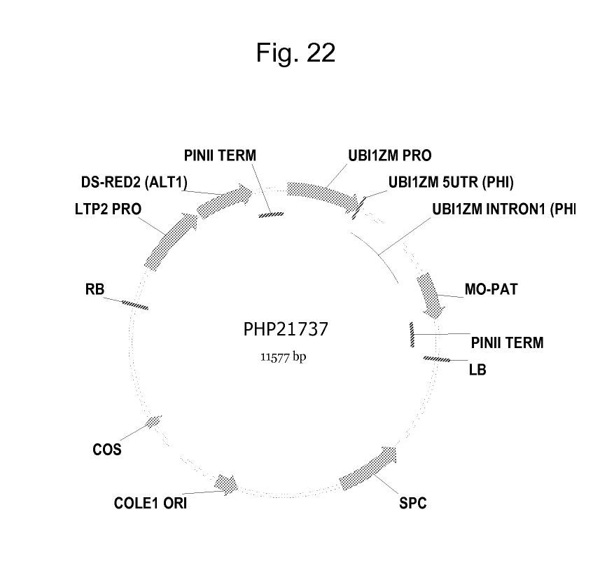

Destination vector PHP28647 (FIG. 19, SEQ ID NO:62) was obtained by transformation of Agrobacterium strain LBA4404 containing plasmid PHP19770 (FIG. 21, SEQ ID NO:64) with plasmid PHP21737 (FIG. 22, SEQ ID NO:65) and isolation of the resulting co-integration product. Destination vector PHP28647, can be used in a recombination reaction with an entry clone as described in Example 16 to create a maize expression vector for transformation of maize lines. Expression of the gene of interest is under control of the ubiquitin promoter (SEQ ID NO:21).

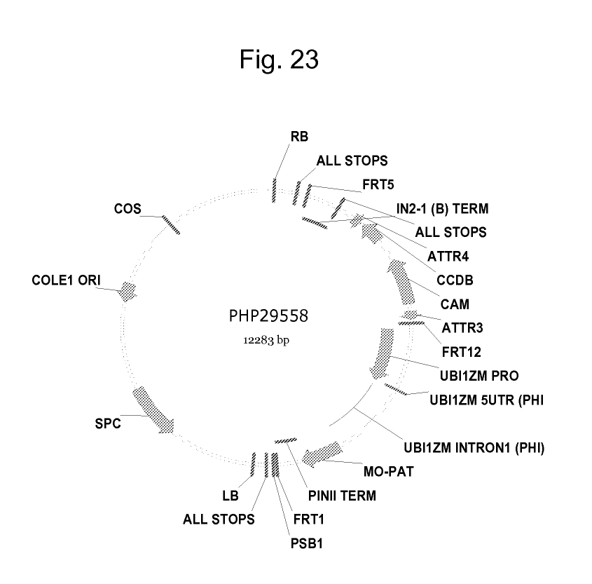

Destination vector PHP33692 was created from plasmid PHP29558 (FIG. 23; SEQ ID NO:66) with a LTP2promoter::red fluorescent protein::PinII terminator cassette, introduced by restriction digestion and ligation. Moreover, destination vector PHP33692 has site-specific recombination sites FRT1, FRT5 and FRT12, as well as LoxP. Destination vector PHP33692 can be used in a recombination reaction with an entry clone as described in Example 16 to create a maize expression vector for transformation of maize lines. Expression of the gene of interest is under control of the NAS2 promoter (SEQ ID NO:19).

### Example 16

### Preparation of Plasmids for Transformation of Gaspe Flint Derived and Other Maize Lines

Using Invitrogen™ Gateway® Recombination technology, entry clones containing the Arabidopsis lpk gene (AT5G60270) or a maize lpk homolog can be created, as described in Examples 5A and 9 and used to directionally clone each gene into destination vector PHP23236 or PHP28647 (Example 15A and B, respectively) for expression under the ubiquitin promoter or into destination vector PHP29635 (Example 15A) for expression under the S2A promoter or into destination vector PHP29634 (Example 15A) or destination vector PHP33692 (example 15B) for expression under the NAS2 promoter. Each of the expression vectors are T-DNA binary vectors for Agrobacterium-mediated transformation into corn.

Gaspe Flint Derived Maize Lines or other maize lines can be transformed with the expression constructs as described in Example 17.

### Example 17

### Transformation of Gaspe Flint Derived Maize Lines or Other Maize Lines with Validated Arabidopsis Lead Genes and Corresponding Homologs from Other Species

Maize plants can be transformed as described in Example 16 to overexpress the Arabidopsis AT5G60270 gene and the corresponding homologs from other species, such as the ones listed in Table 1 and SEQ ID NOs: 47, 48, 49, 50, 51, 52, 53, 54, 55, 56, 57, 58, and 59, in order to examine the resulting phenotype. In addition to the promoters described in Example 16 other promoters such the S2B promoter, the maize ROOTMET2 promoter, the maize Cyclo, the CR1BIO, the CRWAQ81 and the maize ZRP2.4447 are useful for directing expression of lpk genes in maize. Furthermore, a variety of terminators, such as, but not limited to the PINII terminator, can be used to achieve expression of the gene of interest in Gaspe Flint Derived Maize Lines.

Recipient Plants

Recipient plant cells can be from a uniform maize line having a short life cycle (“fast cycling”), a reduced size, and high transformation potential. Typical of these plant cells for maize are plant cells from any of the publicly available Gaspe Flint (GBF) line varieties. One possible candidate plant line variety is the F1 hybrid of GBF×QTM (Quick Turnaround Maize, a publicly available form of Gaspe Flint selected for growth under greenhouse conditions) disclosed in Tomes et al. U.S. Patent Application Publication No. 2003/0221212. Transgenic plants obtained from this line are of such a reduced size that they can be grown in four inch pots (¼ the space needed for a normal sized maize plant) and mature in less than 2.5 months. (Traditionally 3.5 months is required to obtain transgenic T0 seed once the transgenic plants are acclimated to the greenhouse.) Another suitable line is a double haploid line of GS3 (a highly transformable line) X Gaspe Flint. Yet another suitable line is a transformable elite inbred line carrying a transgene which causes early flowering, reduced stature, or both.

Transformation Protocol

Any suitable method may be used to introduce the transgenes into the maize cells, including but not limited to inoculation type procedures using Agrobacterium based vectors as described in Example 9. Transformation may be performed on immature embryos of the recipient (target) plant.

Precision Growth and Plant Tracking

The event population of transgenic (T0) plants resulting from the transformed maize embryos is grown in a controlled greenhouse environment using a modified randomized block design to reduce or eliminate environmental error. A randomized block design is a plant layout in which the experimental plants are divided into groups (e.g., thirty plants per group), referred to as blocks, and each plant is randomly assigned a location with the block.

For a group of thirty plants, twenty-four transformed, experimental plants and six control plants (plants with a set phenotype) (collectively, a “replicate group”) are placed in pots which are arranged in an array (a.k.a. a replicate group or block) on a table located inside a greenhouse. Each plant, control or experimental, is randomly assigned to a location with the block which is mapped to a unique, physical greenhouse location as well as to the replicate group. Multiple replicate groups of thirty plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

An alternative to the addition of a specific control group is to identify those transgenic plants that do not express the gene of interest. A variety of techniques such as RT-PCR can be applied to quantitatively assess the expression level of the introduced gene. T0 plants that do not express the transgene can be compared to those which do.

Each plant in the event population is identified and tracked throughout the evaluation process, and the data gathered from that plant is automatically associated with that plant so that the gathered data can be associated with the transgene carried by the plant. For example, each plant container can have a machine readable label (such as a Universal Product Code (UPC) bar code) which includes information about the plant identity, which in turn is correlated to a greenhouse location so that data obtained from the plant can be automatically associated with that plant.

Alternatively any efficient, machine readable, plant identification system can be used, such as two-dimensional matrix codes or even radio frequency identification tags (RFID) in which the data is received and interpreted by a radio frequency receiver/processor. See U.S. Published Patent Application No. 2004/0122592, incorporated herein by reference.

Phenotypic Analysis Using Three-Dimensional Imaging

Each greenhouse plant in the T0 event population, including any control plants, is analyzed for agronomic characteristics of interest, and the agronomic data for each plant is recorded or stored in a manner so that it is associated with the identifying data (see above) for that plant. Confirmation of a phenotype (gene effect) can be accomplished in the T1 generation with a similar experimental design to that described above.

The T0 plants are analyzed at the phenotypic level using quantitative, non-destructive imaging technology throughout the plant's entire greenhouse life cycle to assess the traits of interest. A digital imaging analyzer is used for automatic multi-dimensional analyzing of total plants. The imaging may be done inside the greenhouse. Two camera systems, located at the top and side, and an apparatus to rotate the plant, are used to view and image plants from all sides. Images are acquired from the top, front and side of each plant. All three images together provide sufficient information to evaluate the biomass, size and morphology of each plant.

Due to the change in size of the plants from the time the first leaf appears from the soil to the time the plants are at the end of their development, the early stages of plant development are best documented with a higher magnification from the top. This may be accomplished by using a motorized zoom lens system that is fully controlled by the imaging software.

In a single imaging analysis operation, the following events occur: (1) the plant is conveyed inside the analyzer area, rotated 360 degrees so its machine readable label can be read, and left at rest until its leaves stop moving; (2) the side image is taken and entered into a database; (3) the plant is rotated 90 degrees and again left at rest until its leaves stop moving, and (4) the plant is transported out of the analyzer.

Plants are allowed at least six hours of darkness per twenty four hour period in order to have a normal day/night cycle.

Imaging Instrumentation

Any suitable imaging instrumentation may be used, including but not limited to light spectrum digital imaging instrumentation commercially available from LemnaTec GmbH of Wurselen, Germany. The images are taken and analyzed with a LemnaTec Scanalyzer HTS LT-0001-2 having a ½″ IT Progressive Scan IEE CCD imaging device. The imaging cameras may be equipped with a motor zoom, motor aperture and motor focus. All camera settings may be made using LemnaTec software. The instrumental variance of the imaging analyzer is less than about 5% for major components and less than about 10% for minor components.

Software

The imaging analysis system comprises a LemnaTec HTS Bonit software program for color and architecture analysis and a server database for storing data from about 500,000 analyses, including the analysis dates. The original images and the analyzed images are stored together to allow the user to do as much reanalyzing as desired. The database can be connected to the imaging hardware for automatic data collection and storage. A variety of commercially available software systems (e.g. Matlab, others) can be used for quantitative interpretation of the imaging data, and any of these software systems can be applied to the image data set.

Conveyor System

A conveyor system with a plant rotating device may be used to transport the plants to the imaging area and rotate them during imaging. For example, up to four plants, each with a maximum height of 1.5 m, are loaded onto cars that travel over the circulating conveyor system and through the imaging measurement area. In this case the total footprint of the unit (imaging analyzer and conveyor loop) is about 5 m×5 m.

The conveyor system can be enlarged to accommodate more plants at a time. The plants are transported along the conveyor loop to the imaging area and are analyzed for up to 50 seconds per plant. Three views of the plant are taken. The conveyor system, as well as the imaging equipment, should be capable of being used in greenhouse environmental conditions.

Illumination

Any suitable mode of illumination may be used for the image acquisition. For example, a top light above a black background can be used. Alternatively, a combination of top- and backlight using a white background can be used. The illuminated area should be housed to ensure constant illumination conditions. The housing should be longer than the measurement area so that constant light conditions prevail without requiring the opening and closing or doors. Alternatively, the illumination can be varied to cause excitation of either transgene (e.g., green fluorescent protein (GFP), red fluorescent protein (RFP)) or endogenous (e.g. Chlorophyll) fluorophores.

Biomass Estimation Based on Three-Dimensional Imaging

For best estimation of biomass the plant images should be taken from at least three axes, such as the top and two side (sides 1 and 2) views. These images are then analyzed to separate the plant from the background, pot and pollen control bag (if applicable). The volume of the plant can be estimated by the calculation:

Volume(voxels)=\sqrt{TopArea(pixels)}×\sqrt{Side1Area(pixels)}×\sqrt{Side2Area(pixels)}

In the equation above the units of volume and area are “arbitrary units”. Arbitrary units are entirely sufficient to detect gene effects on plant size and growth in this system because what is desired is to detect differences (both positive-larger and negative-smaller) from the experimental mean, or control mean. The arbitrary units of size (e.g. area) may be trivially converted to physical measurements by the addition of a physical reference to the imaging process. For instance, a physical reference of known area can be included in both top and side imaging processes. Based on the area of these physical references a conversion factor can be determined to allow conversion from pixels to a unit of area such as square centimeters (cm²). The physical reference may or may not be an independent sample. For instance, the pot, with a known diameter and height, could serve as an adequate physical reference.

Color Classification

The imaging technology may also be used to determine plant color and to assign plant colors to various color classes. The assignment of image colors to color classes is an inherent feature of the LemnaTec software. With other image analysis software systems color classification may be determined by a variety of computational approaches.

For the determination of plant size and growth parameters, a useful classification scheme is to define a simple color scheme including two or three shades of green and, in addition, a color class for chlorosis, necrosis and bleaching, should these conditions occur. A background color class which includes non plant colors in the image (for example pot and soil colors) is also used and these pixels are specifically excluded from the determination of size. The plants are analyzed under controlled constant illumination so that any change within one plant over time, or between plants or different batches of plants (e.g. seasonal differences) can be quantified.

In addition to its usefulness in determining plant size growth, color classification can be used to assess other yield component traits. For these other yield component traits additional color classification schemes may be used. For instance, the trait known as “staygreen”, which has been associated with improvements in yield, may be assessed by a color classification that separates shades of green from shades of yellow and brown (which are indicative of senescing tissues). By applying this color classification to images taken toward the end of the T0 or T1 plants' life cycle, plants that have increased amounts of green colors relative to yellow and brown colors (expressed, for instance, as Green/Yellow Ratio) may be identified. Plants with a significant difference in this Green/Yellow ratio can be identified as carrying transgenes which impact this important agronomic trait.

The skilled plant biologist will recognize that other plant colors arise which can indicate plant health or stress response (for instance anthocyanins), and that other color classification schemes can provide further measures of gene action in traits related to these responses.

### Plant Architecture Analysis

Transgenes which modify plant architecture parameters may also be identified using the present invention, including such parameters as maximum height and width, internodal distances, angle between leaves and stem, number of leaves starting at nodes and leaf length. The LemnaTec system software may be used to determine plant architecture as follows. The plant is reduced to its main geometric architecture in a first imaging step and then, based on this image, parameterized identification of the different architecture parameters can be performed. Transgenes that modify any of these architecture parameters either singly or in combination can be identified by applying the statistical approaches previously described.

Pollen Shed Date

Pollen shed date is an important parameter to be analyzed in a transformed plant, and may be determined by the first appearance on the plant of an active male flower. To find the male flower object, the upper end of the stem is classified by color to detect yellow or violet anthers. This color classification analysis is then used to define an active flower, which in turn can be used to calculate pollen shed date.

Alternatively, pollen shed date and other easily visually detected plant attributes (e.g. pollination date, first silk date) can be recorded by the personnel responsible for performing plant care. To maximize data integrity and process efficiency this data is tracked by utilizing the same barcodes utilized by the LemnaTec light spectrum digital analyzing device. A computer with a barcode reader, a palm device, or a notebook PC may be used for ease of data capture recording time of observation, plant identifier, and the operator who captured the data.

Orientation of the Plants

Mature maize plants grown at densities approximating commercial planting often have a planar architecture. That is, the plant has a clearly discernable broad side, and a narrow side. The image of the plant from the broadside is determined. To each plant a well defined basic orientation is assigned to obtain the maximum difference between the broadside and edgewise images. The top image is used to determine the main axis of the plant, and an additional rotating device is used to turn the plant to the appropriate orientation prior to starting the main image acquisition.

### Example 18A

### Screening of Maize Lines Under Nitrogen Limiting Conditions

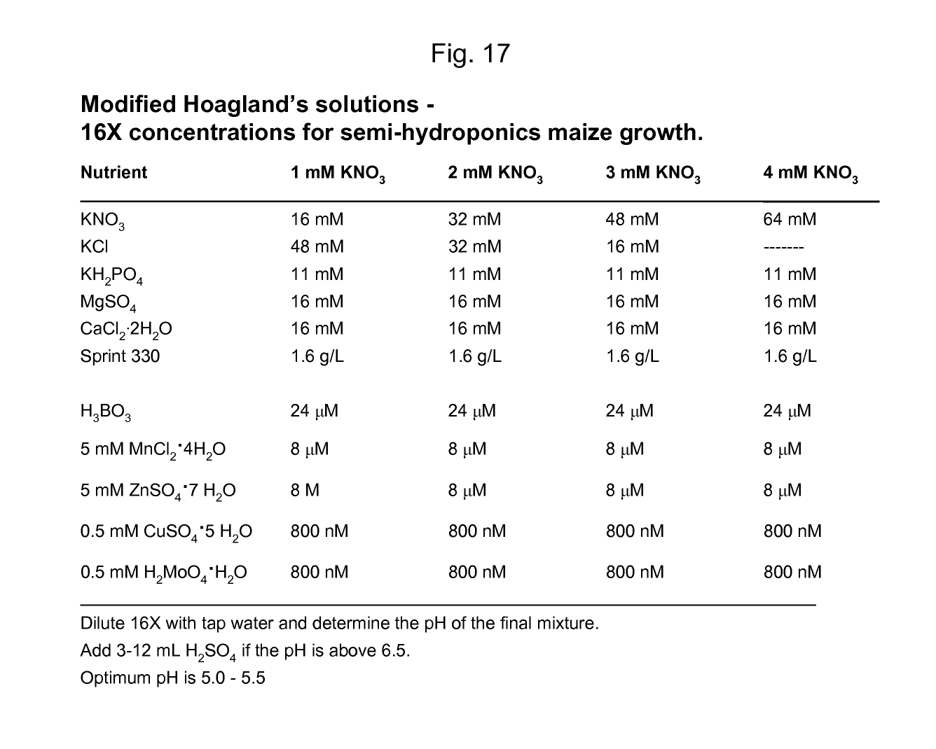

Some transgenic plants will contain two or three doses of Gaspe Flint-3 with one dose of GS3 (GS3/(Gaspe-3)2× or GS3/(Gaspe-3)3×) and will segregate 1:1 for a dominant transgene. Other transgenic plants will be regular inbreds and will be used in top crosses to generate test hybrids. Plants will be planted in Turface, a commercial potting medium, and watered four times each day with 1 mM KNO₃ growth medium and with 2 mM KNO₃, or higher, growth medium (see FIG. 17). Control plants grown in 1 mM KNO₃ medium will be less green, produce less biomass and have a smaller ear at anthesis. Gaspe-derived lines will be grown to flowering stage whereas regular inbreds and hybrids will be grown to V4 to V5 stages.

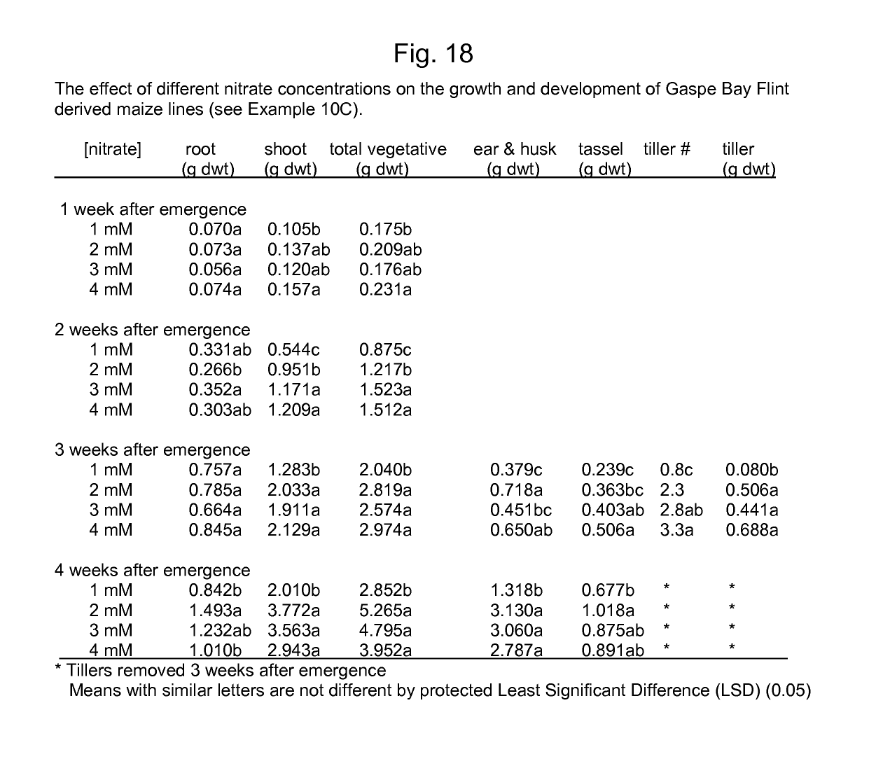

Statistics are used to decide if differences seen between treatments are significantly different. FIG. 18 illustrates one method which places letters after the values. Those values in the same column that have the same letter (not group of letters) following them are not significantly different. Using this method, if there are no letters following the values in a column, then there are no significant differences between any of the values in that column or, in other words, all the values in that column are equal. Expression of a transgene will result in plants with improved plant growth in 1 mM KNO₃ when compared to a transgenic null. Thus biomass and greenness data (as described in Example 17) will be collected at time of sampling (anthesis for Gaspe and V4-V5 for others) and compared to a transgenic null. In addition, total nitrogen in the plants will be analyzed in ground tissues. Improvements in growth, greenness, nitrogen accumulation and ear size at anthesis will be indications of increased nitrogen use efficiency.

Seedling Assay

Transgenic maize plants can also be evaluated using a seedling assay that assesses plant performance under nitrogen limiting conditions. In an 18 day seedling assay, for example, transgenic plants are planted in Turface, a commercial potting medium, and then watered four times each day with a solution containing the following nutrients: 1 mM CaCl₂, 2 mM MgSO₄, 0.5 mM KH₂PO₄, 83 ppm Sprint330, 3 mM KCl, 1 mM KNO₃, 1 μM ZnSO₄, 1 μM MnCl₂, 3 μM H₃BO₄, 0.1 μM CuSO₄, and 0.1 μM NaMoO₄. Plants are harvested 18 days after planting, and a number of traits are assessed, including but not limited to: SPAD (greenness), stem diameter, root dry weight, shoot dry weight, total dry weight, mg Nitrogen per grams of dry weight (mg N/g dwt), and plant N concentration. Means are compared to null mean parameters using a Student's t test with a minimum (P<t) of 0.1.

### Example 18B

### Nitrogen Utilization Efficiency Seedling Assay

Seed of transgenic events were separated into Transgenic (Treatment 1; contains construct PHP31272) and Null (Treatment 2) seed using a seed color marker.

Treatments (Transgenic or Bulked Null) were each randomly assigned to blocks of 54 pots (experimental units) arranged in 6 rows by 9 columns. Each treatment (Transgenic or Bulked Nulls) was replicated 9 times.

All seeds were planted in 4 inch, square pots containing Turface on 8 inch, staggered centers and watered four times each day with a solution containing the following nutrients:

| | | | |
|---|---|---|---|
| 1 mM CaCl₂ | 2 mM MgSO₄ | 0.5 mM KH₂PO₄ | 83 ppm Sprint330 |
| 3 mM KCl | 1 mM KNO₃ | 1 μM ZnSO₄ | 1 μM MnCl₂ |
| 3 μM H₃BO₄ | 1 μM MnCl₂ | 0.1 μM CuSO₄ | 0.1 μM NaMoO₄ |

After emergence the plants were thinned to one seed per pot. At harvest, plants were removed from the pots, and the Turface was washed from the roots. The roots were separated from the shoot, placed in a paper bag, and dried at 70° C. for 70 hr. The dried plant parts (roots and shoots) were weighed and placed in a 50 ml conical tube with approximately 20 5/32 inch steel balls and then ground by shaking in a paint shaker.

The Nitrogen/Protein Analyzer from Thermo Electron Corporation (model FlashEA 1112 N) uses approximately 30 mg of the ground tissue. A sample is dropped from the Autosampler into the crucible inside the oxidation reactor chamber. At 900° C. and pure oxygen, the sample is oxidized by a strong exothermic reaction creating a gas mixture of N₂, CO₂, H₂O, and SO₂. After the combustion is complete, the carrier gas helium is turned on and the gas mixture flows into the reduction reaction chamber. At 680° C., the gas mixture flows across the reduction copper where nitrogen oxides possibly formed are converted into elemental nitrogen and the oxygen excess is retained. From the reduction reactor, the gas mixture flows across a series of two absorption filters. The first filter contains soda lime and retains carbon and sulfur dioxides. The second filter contains molecular sieves and granular silica gel to hold back water. Nitrogen is then eluted in the chromatographic column and conveyed to the thermal conductivity detector that generates an electrical signal, which, properly processed by the Eager 300 software, provides the nitrogen-protein percentage.

Using these data, the following parameters were measured and means of Transgenic parameters were compared to means of Null parameters using a Student's t test:

| | |
|---|---|
| Total Plant Biomass | (total dwt (g)) |
| Root Biomass | (root dwt (g)) |
| Shoot Biomass | (shoot dwt (g)) |
| Root/Shoot Ratio | (root:shoot dwt ratio) |
| Plant N concentration | (mg N/g dwt) |
| Total Plant N | (total N (mg)) |

Variance was calculated within each block using an Analysis of Variance (ANOVA) calculation and a completely random design (CRD) model. An overall treatment effect for each block was calculated using an F statistic by dividing overall block treatment mean square by the overall block error mean square. The probability of a greater Student's t test was calculated for each transgenic mean compared to the appropriate null. Variables that show a significant difference (*) have a minimum (P<t) of 0.1.

Table 5 shows the data and the two tailed Student's t probability for plants containing construct PHP31272. Comparisons were made between the transgenic events and construct nulls planted on 9/1. A construct null is a negative entry that is made up of a sampling of kernels from the negative segregants and is therefore a representative sample of all negatives.

TABLE 5
Greenhouse Seedling Assay Data for PHP31272

| Planting | | | | | | | |
|---|---|---|---|---|---|---|---|
| date | Event Name | Mean | Null | t Test | Mean | Null | t Test |
| | | root dwt (g) | | | shoot dwt (g) | | |
| Sep. 1, 2010 | E7899.41.1.1 | 0.373 | 0.421 | 0.116 | 0.59 | 0.612 | 0.662 |
| Sep. 1, 2010 | E7899.41.1.3 | 0.403 | 0.405 | 0.945 | 0.539 | 0.518 | 0.676 |
| Sep. 1, 2010 | E7899.41.1.4 | 0.396 | 0.341 | 0.064 | 0.575 | 0.511 | 0.204 |
| Sep. 1, 2010 | E7899.41.2.13 | 0.399 | 0.378 | 0.476 | 0.559 | 0.574 | 0.765 |
| Sep. 1, 2010 | E7899.41.2.18 | 0.39 | 0.405 | 0.609 | 0.51 | 0.561 | 0.311 |
| Sep. 1, 2010 | E7899.41.2.2 | 0.43 | 0.434 | 0.892 | 0.617 | 0.555 | 0.226 |
| Sep. 1, 2010 | E7899.41.2.4 | 0.438 | 0.45 | 0.684 | 0.542 | 0.58 | 0.450 |
| Sep. 1, 2010 | E7899.41.4.9 | 0.426 | 0.39 | 0.260 | 0.52 | 0.543 | 0.647 |
| Sep. 1, 2010 | E7899.41.5.2 | 0.438 | 0.426 | 0.798 | 0.56 | 0.587 | 0.606 |
| Sep. 1, 2010 | E7899.41.1.1 | 0.542 | 0.559 | 0.711 | 1.312 | 1.364 | 0.636 |
| Sep. 1, 2010 | E7899.41.1.3 | 0.554 | 0.6 | 0.316 | 1.469 | 1.39 | 0.470 |
| Sep. 1, 2010 | E7899.41.1.4 | 0.604 | 0.606 | 0.980 | 1.434 | 1.513 | 0.473 |
| Sep. 1, 2010 | E7899.41.2.13 | 0.621 | 0.534 | 0.075 | 1.462 | 1.386 | 0.511 |
| Sep. 1, 2010 | E7899.41.2.18 | 0.615 | 0.497 | 0.015 | 1.28 | 1.332 | 0.643 |
| Sep. 1, 2010 | E7899.41.2.2 | 0.662 | 0.587 | 0.139 | 1.81 | 1.478 | 0.003 |
| Sep. 1, 2010 | E7899.41.2.4 | 0.586 | 0.591 | 0.913 | 1.37 | 1.298 | 0.558 |
| Sep. 1, 2010 | E7899.41.4.9 | 0.588 | 0.518 | 0.145 | 1.511 | 1.381 | 0.239 |
| Sep. 1, 2010 | E7899.41.5.2 | 0.553 | 0.588 | 0.445 | 1.307 | 1.39 | 0.451 |
| Sep. 1, 2010 | E7899.41.1.1 | 0.494 | 0.553 | 0.058 | 1.165 | 1.207 | 0.649 |
| Sep. 1, 2010 | E7899.41.1.3 | 0.431 | 0.49 | 0.076 | 1.007 | 1.079 | 0.436 |
| Sep. 1, 2010 | E7899.41.1.4 | 0.446 | 0.505 | 0.086 | 1.026 | 1.269 | 0.010 |
| Sep. 1, 2010 | E7899.41.2.13 | 0.475 | 0.491 | 0.581 | 1.137 | 1.206 | 0.456 |
| Sep. 1, 2010 | E7899.41.2.18 | 0.471 | 0.486 | 0.655 | 1.174 | 1.153 | 0.820 |
| Sep. 1, 2010 | E7899.41.2.2 | 0.528 | 0.5 | 0.335 | 1.217 | 1.213 | 0.936 |
| Sep. 1, 2010 | E7899.41.2.4 | 0.472 | 0.477 | 0.863 | 1.085 | 1.065 | 0.828 |
| Sep. 1, 2010 | E7899.41.4.9 | 0.487 | 0.511 | 0.446 | 1.071 | 1.165 | 0.341 |
| Sep. 1, 2010 | E7899.41.5.2 | 0.477 | 0.43 | 0.107 | 1.138 | 1.117 | 0.786 |
| | | total dwt (g) | | | root:shoot dwt ratio | | |
| Sep. 1, 2010 | E7899.41.1.1 | 0.987 | 1.033 | 0.531 | 0.678 | 0.695 | 0.685 |
| Sep. 1, 2010 | E7899.41.1.3 | 0.942 | 0.923 | 0.795 | 0.773 | 0.800 | 0.512 |
| Sep. 1, 2010 | E7899.41.1.4 | 0.971 | 0.852 | 0.107 | 0.694 | 0.674 | 0.639 |
| Sep. 1, 2010 | E7899.41.2.13 | 0.958 | 0.952 | 0.934 | 0.730 | 0.667 | 0.149 |
| Sep. 1, 2010 | E7899.41.2.18 | 0.881 | 0.977 | 0.209 | 0.769 | 0.813 | 0.328 |
| Sep. 1, 2010 | E7899.41.2.2 | 1.061 | 0.989 | 0.349 | 0.715 | 0.807 | 0.040 |
| Sep. 1, 2010 | E7899.41.2.4 | 0.98 | 0.968 | 0.869 | 0.813 | 0.782 | 0.463 |
| Sep. 1, 2010 | E7899.41.4.9 | 0.922 | 0.933 | 0.880 | 0.785 | 0.719 | 0.126 |
| Sep. 1, 2010 | E7899.41.5.2 | 0.998 | 1.013 | 0.825 | 0.789 | 0.730 | 0.218 |
| Sep. 1, 2010 | E7899.41.1.1 | 1.829 | 1.923 | 0.541 | 0.401 | 0.406 | 0.816 |
| Sep. 1, 2010 | E7899.41.1.3 | 2.023 | 2.044 | 0.891 | 0.357 | 0.413 | 0.023 |
| Sep. 1, 2010 | E7899.41.1.4 | 2.02 | 2.149 | 0.402 | 0.411 | 0.427 | 0.501 |
| Sep. 1, 2010 | E7899.41.2.13 | 2.188 | 1.92 | 0.091 | 0.383 | 0.387 | 0.861 |
| Sep. 1, 2010 | E7899.41.2.18 | 1.873 | 1.854 | 0.909 | 0.434 | 0.377 | 0.022 |
| Sep. 1, 2010 | E7899.41.2.2 | 2.504 | 2.096 | 0.008 | 0.385 | 0.397 | 0.603 |
| Sep. 1, 2010 | E7899.41.2.4 | 1.956 | 1.872 | 0.623 | 0.421 | 0.423 | 0.884 |
| Sep. 1, 2010 | E7899.41.4.9 | 2.099 | 1.86 | 0.131 | 0.391 | 0.404 | 0.595 |
| Sep. 1, 2010 | E7899.41.5.2 | 1.86 | 1.978 | 0.443 | 0.424 | 0.425 | 0.958 |
| Sep. 1, 2010 | E7899.41.1.1 | 1.672 | 1.742 | 0.545 | 0.439 | 0.455 | 0.442 |
| Sep. 1, 2010 | E7899.41.1.3 | 1.438 | 1.558 | 0.300 | 0.451 | 0.445 | 0.774 |
| Sep. 1, 2010 | E7899.41.1.4 | 1.472 | 1.782 | 0.008 | 0.427 | 0.405 | 0.305 |
| Sep. 1, 2010 | E7899.41.2.13 | 1.612 | 1.697 | 0.462 | 0.423 | 0.394 | 0.253 |
| Sep. 1, 2010 | E7899.41.2.18 | 1.665 | 1.578 | 0.464 | 0.412 | 0.422 | 0.668 |
| Sep. 1, 2010 | E7899.41.2.2 | 1.738 | 1.718 | 0.851 | 0.422 | 0.419 | 0.962 |
| Sep. 1, 2010 | E7899.41.2.4 | 1.557 | 1.542 | 0.896 | 0.440 | 0.427 | 0.682 |
| Sep. 1, 2010 | E7899.41.4.9 | 1.558 | 1.676 | 0.338 | 0.453 | 0.442 | 0.610 |
| Sep. 1, 2010 | E7899.41.5.2 | 1.615 | 1.551 | 0.548 | 0.435 | 0.372 | 0.010 |
| | | mg N/g dwt | | | total N (mg) | | |
| Sep. 1, 2010 | E7899.41.1.1 | 17.56 | 18.48 | 0.233 | 10.383 | 11.283 | 0.237 |
| Sep. 1, 2010 | E7899.41.1.3 | 19.67 | 18.51 | 0.105 | 11.967 | 09.520 | 0.000 |
| Sep. 1, 2010 | E7899.41.1.4 | 19.4 | 20.01 | 0.428 | 11.176 | 10.395 | 0.285 |
| Sep. 1, 2010 | E7899.41.2.13 | 17.89 | 18.61 | 0.350 | 10.476 | 10.593 | 0.853 |
| Sep. 1, 2010 | E7899.41.2.18 | 19.05 | 17.62 | 0.065 | 09.658 | 09.147 | 0.524 |
| Sep. 1, 2010 | E7899.41.2.2 | 17.66 | 17.41 | 0.745 | 10.228 | 09.655 | 0.474 |
| Sep. 1, 2010 | E7899.41.2.4 | 18.75 | 19.75 | 0.195 | 10.100 | 11.211 | 0.145 |
| Sep. 1, 2010 | E7899.41.4.9 | 19.42 | 19.59 | 0.825 | 10.040 | 10.574 | 0.482 |
| Sep. 1, 2010 | E7899.41.5.2 | 17.72 | 18.54 | 0.425 | 10.184 | 11.124 | 0.275 |
| Sep. 1, 2010 | E7899.41.1.1 | 42.06 | 41.5 | 0.567 | 53.532 | 54.703 | 0.763 |
| Sep. 1, 2010 | E7899.41.1.3 | 40.44 | 41.37 | 0.426 | 58.946 | 59.222 | 0.943 |
| Sep. 1, 2010 | E7899.41.1.4 | 41.47 | 41.02 | 0.757 | 59.272 | 60.254 | 0.800 |
| Sep. 1, 2010 | E7899.41.2.13 | 40.58 | 42.01 | 0.221 | 64.020 | 58.205 | 0.145 |
| Sep. 1, 2010 | E7899.41.2.18 | 41.02 | 42.13 | 0.342 | 52.228 | 55.906 | 0.358 |
| Sep. 1, 2010 | E7899.41.2.2 | 40.15 | 40.41 | 0.724 | 74.094 | 59.480 | 0.0003 |
| Sep. 1, 2010 | E7899.41.2.4 | 40.76 | 42.41 | 0.216 | 55.420 | 59.619 | 0.282 |
| Sep. 1, 2010 | E7899.41.4.9 | 40.72 | 40.35 | 0.761 | 60.976 | 55.439 | 0.166 |
| Sep. 1, 2010 | E7899.41.5.2 | 42.26 | 42.45 | 0.961 | 55.202 | 56.859 | 0.681 |
| Sep. 1, 2010 | E7899.41.1.1 | 48.97 | 48.77 | 0.821 | 56.185 | 59.121 | 0.491 |
| Sep. 1, 2010 | E7899.41.1.3 | 50.01 | 50.22 | 0.814 | 50.074 | 54.124 | 0.343 |
| Sep. 1, 2010 | E7899.41.1.4 | 49.98 | 49.14 | 0.357 | 51.739 | 62.118 | 0.016 |
| Sep. 1, 2010 | E7899.41.2.13 | 50.38 | 49.88 | 0.581 | 56.272 | 60.068 | 0.374 |
| Sep. 1, 2010 | E7899.41.2.18 | 50.61 | 49.51 | 0.221 | 59.342 | 54.940 | 0.315 |
| Sep. 1, 2010 | E7899.41.2.2 | 48.78 | 48.97 | 0.830 | 59.166 | 57.761 | 0.740 |
| Sep. 1, 2010 | E7899.41.2.4 | 50.34 | 50.98 | 0.514 | 54.410 | 53.979 | 0.919 |
| Sep. 1, 2010 | E7899.41.4.9 | 50.1 | 50.05 | 0.955 | 54.203 | 58.306 | 0.365 |
| Sep. 1, 2010 | E7899.41.5.2 | 50.05 | 51 | 0.290 | 56.175 | 57.152 | 0.851 |

### Example 19A

### Yield Analysis of Maize Lines with Validated Arabidopsis Lead Gene (AT5G60270)

A recombinant DNA construct containing a validated Arabidopsis gene can be introduced into a maize line either by direct transformation or introgression from a separately transformed line.

Transgenic plants, either inbred or hybrid, can undergo more vigorous field-based experiments to study yield enhancement and/or stability under various environmental conditions, such as variations in water and nutrient availability (e.g. nitrogen limiting and non-limiting conditions). A standardized yield trial will typically include 4 to 6 replications and at least 4 locations.

Subsequent yield analysis can be done to determine whether plants that contain the validated Arabidopsis lead gene have an improvement in yield performance under various environmental conditions (e.g. under nitrogen limiting or non-limiting conditions), when compared to the control plants that do not contain the validated Arabidopsis lead or a maize homolog of the gene. Reduction in yield can be measured for both. Plants containing the validated Arabidopsis lead gene have less yield loss relative to the control plants, for example 50% less yield loss.

### Example 19B

### Yield Analysis of Maize Lines Transformed with PHP31272 and PHP37086

Corn hybrid testcrosses, containing the Arabidopsis thaliana lead gene (encoding a lectin protein kinase polypeptide) expression cassette present in vector PHP31272 and PHP37086, and their controls were grown in low nitrogen (LN) and normal nitrogen (NN) environments at multiple locations. A low nitrogen (LN) environment consists of a less than normal amount of nitrogen fertilizer applied in early spring or summer, whereas a normal nitrogen (NN) environment consists of adding adequate nitrogen for normal yields, based on soil test standards established for specific growing areas by Federal and State Extension services. For the analysis, a construct null was a negative entry made up of negative segregants from all events within a construct, and a bulk null was a negative entry made up of all negative segregants from all constructs within an experiment.

For PHP31272 nine transgenic events were field tested for LN and NN in 2008 at two locations (LN2 and NN2, respectively), York, Nebr. (YK) and Woodland, Calif. (WO). In 2009 the nine transgenic events were field tested in three locations (LN3), Marion, Iowa (MR), York, Nebr. (YK) and Woodland, Calif. (WO), for LN and at four locations (NN4), Marion, Iowa (MR), York, Nebr. (YK), Woodland, Calif. (WO), and Princeton, Ill. (PR) for NN. In 2010 the nine transgenic events were field tested for three locations (LN3) for LN and at two locations (NN2) for NN. For PHP37086 ten transgenic events were field tested for LN and NN in 2010 at three locations for LN and at two locations for NN. Yield was assessed and compared and calculated across locations for each year. The corn hybrid testcrosses were compared to the nulls (either bulk null or construct null). The summary of the results of the field tests are presented in Table 6 (PHP31272) and Table 7 (PHP37086).

TABLE 6
2008, 2009 and 2010 Field Tests of Maize Transformed with PHP31272

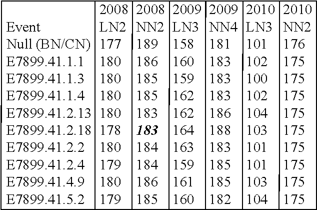

Unit of measure is bushels/acre
Shading represents sig. higher (P < 0.1) results compared to the null.
Bold represents significant lower (P < 0.1) result compared to the null.

TABLE 7
2010 Field Test of Maize Transformed with PHP37086

| | MR | WO | JH | LN3 | MR NN | YK | NN2 |
|---|---|---|---|---|---|---|---|
| Event | LN | LN | LN | | | NN | |
| BN | 104 | 112 | 97 | 104 | 179 | 188 | 184 |
| E8828-47.1.19 | 104 | 110 | 97 | 104 | 178 | 187 | 184 |
| E8828-47.1.20 | 104 | 109 | 97 | 104 | 178 | 186 | 182 |
| E8828-47.1.5 | 104 | 108 | 97 | 104 | 180 | 186 | 183 |
| E8828-47.2.15 | 104 | 110 | 97 | 104 | 180 | 187 | 184 |
| E8828-47.2.19 | 104 | 111 | 97 | 104 | 177 | 187 | 182 |
| E8828-47.2.29 | 104 | 109 | 97 | 104 | 178 | 186 | 182 |
| E8828-47.2.6 | 104 | 108 | 97 | 104 | 177 | 186 | 182 |
| E8828-47.3.10 | 104 | 109 | 97 | 104 | 178 | 187 | 182 |
| E8828-47.3.13 | 104 | 110 | 97 | 104 | 178 | 186 | 182 |
| E8828-47.3.4 | 104 | 110 | 97 | 104 | 178 | 187 | 183 |

Unit of measure is bushels/acre.
Shading represents sig. higher (P < 0.1) result compared to the construct null (CN).
Bold represents sig. lower (P < 0.1) result compared to the construct null (CN).

### Example 20

### Assays to Determine Alterations of Root Architecture in Maize

Transgenic maize plants are assayed for changes in root architecture at seedling stage, flowering time or maturity. Assays to measure alterations of root architecture of maize plants include, but are not limited to the methods outlined below. To facilitate manual or automated assays of root architecture alterations, corn plants can be grown in clear pots.
1) Root mass (dry weights). Plants are grown in Turface, a growth media that allows easy separation of roots. Oven-dried shoot and root tissues are weighed and a root/shoot ratio calculated.
2) Levels of lateral root branching. The extent of lateral root branching (e.g. lateral root number, lateral root length) is determined by sub-sampling a complete root system, imaging with a flat-bed scanner or a digital camera and analyzing with WinRHIZO™ software (Regent Instruments Inc.).
3) Root band width measurements. The root band is the band or mass of roots that forms at the bottom of greenhouse pots as the plants mature. The thickness of the root band is measured in mm at maturity as a rough estimate of root mass.
4) Nodal root count. The number of crown roots coming off the upper nodes can be determined after separating the root from the support medium (e.g. potting mix). In addition the angle of crown roots and/or brace roots can be measured. Digital analysis of the nodal roots and amount of branching of nodal roots form another extension to the aforementioned manual method.
   All data taken on root phenotype are subjected to statistical analysis, normally a t-test to compare the transgenic roots with that of non-transgenic sibling plants. One-way ANOVA may also be used in cases where multiple events and/or constructs are involved in the analysis.

### Example 21

### Association Mapping Analysis

An association mapping strategy can be undertaken to identify markers associated with alterations in root architecture in maize.

Phenotypic scores for an alteration in root architecture or in at least one agronomic characteristic will be obtained. Lines with extreme phenotypes will be tested against genotypes in a whole genome association test (using 2×2 contingency tables with Fisher's exact test). A structure-based association analysis will be used, where the population structure is controlled using marker data. The model-based cluster analysis software, Structure, developed by Pritchard et al., (Genetics 155:945-959 (2000)) will be used with haplotype data for hundreds of elite maize inbreds at several hundred markers to estimate admixture coefficients and assign the inbreds to a number of subpopulations. This reduces the occurrence of false positives that can arise due to the effect of population structure on association mapping statistics. Kuiper's statistic for testing whether two distributions are the same was used to test a given marker for association between haplotype and phenotype in a given subpopulation (Press et al., Numerical Recipes in C, second edition, Cambridge University Press, NY (2002)).

At least one strong peak in at least one subpopulation is indicative of significant marker-trait associations (e.g. p<0.001). Marker positions are given in cM, with position zero being the first (most distal from the centromere) marker known at the beginning of a chromosome. These map positions are not absolute, and represent an estimate of map position based on the internally derived genetic map.

What is claimed is:

1. A plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, and wherein said plant exhibits altered root architecture when compared to a control plant not comprising said recombinant DNA construct.

2. A plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, and wherein said plant exhibits an alteration in yield, biomass, root lodging or root architecture, or any combination thereof when compared to a control plant not comprising said recombinant DNA construct.

3. The plant of claim 2, wherein the plant exhibits said alteration in yield, biomass, root lodging or root architecture, or any combination thereof, when compared, under varying environmental condition to a control plant not comprising said recombinant DNA construct and wherein said environmental condition is at least one selected from drought, nitrogen, soil type, insect and disease.

4. The plant of any one of claims 1 to 3, wherein said plant is selected from the group consisting of: maize, soybean, sunflower, sorghum, canola, wheat, alfalfa, cotton, rice barley, millet, sugar cane and switchgrass.

5. Seed of the plant of any one of claims 1 to 4, wherein said seed comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70, and wherein a plant produced from said seed exhibits an alteration in at least one trait selected from the group consisting of: root architecture, root lodging, yield and biomass, when compared to a control plant not comprising said recombinant construct.

6. A method of altering root architecture in a plant, comprising:
(a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70;
(b) regenerating a transgenic plant from the regenerable plant cell after step (a), wherein the transgenic plant comprises in its genome the recombinant DNA construct; and
(c) obtaining a progeny plant derived from the transgenic plant of step (b), wherein said progeny plant comprises in its genome the recombinant DNA construct and exhibits an alteration in root architecture when compared to a control plant not comprising the recombinant DNA construct.

7. A method of evaluating alteration in root architecture in a plant, comprising:
(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70;
(b) obtaining a progeny plant derived from the transgenic plant, wherein the progeny plant comprises in its genome the recombinant DNA construct; and
(c) evaluating the progeny plant for alteration in root architecture compared to a control plant not comprising the recombinant DNA construct.

8. A method of determining an alteration of yield, biomass or root architecture, or a combination thereof in a plant, comprising:
(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 27, 29, 35, 37, 39, 41, 43, 46, 47, 48, 49, 50, 51, 52, 53, 54, 55, 56, 57, 58, 59, 68 or 70;
(b) obtaining a progeny plant derived from the transgenic plant, wherein the progeny plant comprises in its genome the recombinant DNA construct; and
(c) determining whether the progeny plant exhibits an alteration of yield, biomass, or root architecture or a combination thereof when compared to a control plant not comprising the recombinant DNA construct.

9. The method of claim 8, wherein said determining step © comprises determining whether the transgenic exhibits an alteration of yield, biomass or root architecture or a combination thereof when compared, under varying environmental condition, wherein the varying environmental condition is at least one selected from drought, nitrogen, soil type, insect or disease., to a control plant not comprising the recombinant DNA construct.

10. The method of claim 8 or 9, wherein said alteration in biomass or yield is an increase.

11. The method of any one of claims 6 to 10, wherein said plant is selected from the group consisting of: maize, soybean, sunflower, sorghum, canola, wheat, alfalfa, cotton, rice, barley millet, sugar cane and switchgrass.

12. An isolated polynucleotide comprising:
(i) a nucleic acid sequence encoding a polypeptide having an amino acid sequence of at least 85%, sequence identity, based on the Clustal V method of alignment, when compared to SEQ ID NO: 35 or (ii) a full complement of the nucleic acid sequence of (i).

13. The polynucleotide of claim 12, wherein the amino acid sequence of the polypeptide comprises SEQ ID NO: 35.

14. The polynucleotide of claim 12 wherein the nucleotide sequence comprises SEQ ID NO: 34.

15. A plant or seed comprising a recombinant DNA construct, wherein the recombinant DNA construct comprises the polynucleotide of any one of claims 12 to 14 operably linked to at least one regulatory sequence.
